(19) United States
(12) Patent Application Publication
(10) Pub. No.: US 2013/0023714 A1
(43) Pub. Date: Jan. 24, 2013

(54) Medical and Imaging Nanoclusters

(71) Applicants: Keith P. Johnston, Austin, TX (US); Li L. Ma, Austin, TX (US); Marc D. Feldman, San Antonio, TX (US); Thomas E. Milner, Austin, TX (US); Konstantin V. Sokolov, Austin, TX (US); Jasmine Rowe, Austin, TX (US); Justina Tam, Austin, TX (US); Stanislav Emelianov, Austin, TX (US); Kort Travis, Austin, TX (US); Avinash K. Murthy, Austin, TX (US)

(72) Inventors: Keith P. Johnston, Austin, TX (US); Li L. Ma, Austin, TX (US); Marc D. Feldman, San Antonio, TX (US); Thomas E. Milner, Austin, TX (US); Konstantin V. Sokolov, Austin, TX (US); Jasmine Rowe, Austin, TX (US); Justina Tam, Austin, TX (US); Stanislav Emelianov, Austin, TX (US); Kort Travis, Austin, TX (US); Avinash K. Murthy, Austin, TX (US)

(73) Assignee: BOARD OF REGENTS, THE UNIVERSITY OF TEXAS SYSTEMS, Austin, TX (US)

(21) Appl. No.: 13/125,565

(22) PCT Filed: Oct. 26, 2009
(86) PCT No.: PCT/US2009/062102

§ 371 Date: Mar. 29, 2012

Related U.S. Application Data
(60) Provisional application No. 61/108,530, filed on Oct. 26, 2008.
(60) Provisional application No. 61/167,329, filed on Apr. 7, 2009.

(51) Int. Cl. A61K 9/14; G01N 24/00; G01N 29/06; A61K 49/08; A61P 35/00; A61P 9/10; A61K 31/519; A61K 38/16; A61K 31/7105; A61K 39/395; A61K 31/713; A61M 37/00; A61B 6/00; A61B 5/055; A61B 8/00; G01N 21/00; B82Y 5/00; B82Y 15/00

(52) U.S. Cl. 600/1; 435/29; 424/400; 424/9.32; 424/490; 514/249; 514/1.9; 514/19.3; 514/44 R; 424/130.1; 514/44 A; 604/20; 600/13; 600/9; 600/431; 600/420; 600/425; 600/407; 977/906; 977/927; 977/774; 977/929; 977/930

(57) ABSTRACT

In one embodiment the present invention discloses a nanocluster or a nanorose composition comprising two or more closely spaced nanoparticles each comprising one or more metals, metal oxides, inorganic substances, or a combination thereof and one or more stabilizers. The stabilizers are in contact with the two or more closely spaced nanoparticles to form a nanocluster composition in which the inorganic weight percentage is greater than 50% and the average size is below 300 nm, and the nanocluster composition has magnetic properties, optical properties or a combination of both.

### TECHNICAL FIELD OF THE INVENTION

The present invention relates in general to the field of nanoclusters of metal nanoparticles, particularly gold nanoparticles, composite nanoclusters of metals and metal oxide nanoparticles and more particularly, to compositions, methods, and applications of nanoclusters stabilized by small amounts of polymers including biocompatible or biodegradable polymers.

### BACKGROUND ART

Without limiting the scope of the invention, its background is described in connection with medical uses for gold or gold-coated nanoparticles where the nanocluster is optimized to have enhanced NIR absorbance. It will be understood that in general, other types of primary nanoparticle, both organic and inorganic, and possibly optimized for other types of electromagnetic interaction are also described by the invention.

Many current efforts include development of targeted gold nanocomposites as contrast agents in near infrared (NIR) region for optical imaging (optical coherence tomography, photoacoustic tomography, and two-photon luminescence), and as photothermal agents for cancer treatment. For deeper tissues in vivo imaging and therapeutic treatment, the optical resonance of nanoparticles is strongly desired to be in the near infrared region (650-900 nm), where the major absorbers of visible light, hemoglobin water and body tissues, have the lowest absorption coefficient. The effectiveness of NIR functionalized nanocomposites as biomedical imaging contrast agents and photothermal therapies not only depends on particle scattering or absorption cross-section at certain interested NIR light wavelength, but also strongly relies on nanoparticle size and surface coating determined targeting and uptake rate by cells. The biocompatibility and toxicity of the nanocomposites have also been addressed as the major drawback for certain nanoparticles. The stability of the nanoparticles in different physiological environments has not been emphasized, which are also crucial for the final products commercialization.

Gold nanomaterials have intrinsic problems based on the consideration of effectiveness, toxicity and stability discussed on the above. For example, colloidal gold nanosphere dispersions do not have a strong surface plasmon resonance peak in NIR region compared with the gold nanorods, nanoshells and nanocages. To synthesis high quality gold nanorods, a strong capping ligand cetyl trimethylammonium bromide (CTAB) and a mediation agent AgNO₃ were used, which are toxic and very difficult to be removed from the surface of nanorods.

### DISCLOSURE OF THE INVENTION

In one embodiment the present invention discloses a nanocluster composition comprising two or more closely spaced nanoparticles each comprising one or more metals, metal oxides, inorganic substances, or a combination thereof and one or more stabilizers. The stabilizers are in contact with the two or more closely spaced nanoparticles to form a nanocluster composition in which the inorganic weight percentage is greater than 50% and the average size is below 300 nm, and the nanocluster composition has magnetic properties, optical properties or a combination of both.

In another embodiment the present invention describes a medical biodegradable nanocluster composition comprising, two or more nanoparticles each comprising one or more metals, metal oxides, inorganic substances, or a combination thereof and one or more stabilizers in contact with the two or more nanoparticles to form a biodegradable nanocluster composition in which an inorganic weight percentage is greater than 50% and the average size is below 300 nm. The medical biodegradable nanocluster composition has magnetic properties, optical properties or a combination of both. In addition the medical biodegradable nanocluster of the present invention may optionally contain one or more active agents in contact with the two or more nanoparticles, wherein the one or more active agents are enclosed within the biodegradable nanocluster, on the surface of the biodegradable nanocluster or both.

In yet another embodiment the present invention is a method forming an optionally biodegradable nanocluster composition comprising the steps of: (i) forming an aqueous dispersion comprising two or more nanoparticles and one or more stabilizers in a solvent and (ii) aggregating the two or more nanoparticles and the one or more stabilizers to form a biodegradable nanocluster composition, in which an inorganic weight percentage is greater than 50% and the average size is below 300 nm, wherein the biodegradable nanocluster composition has magnetic properties, optical properties or a combination of both.

In one embodiment the present invention describes a method for imaging comprising the steps of: providing a sample, administering one or more biodegradable nanocluster compositions to the sample, and imaging the one or more biodegradable nanocluster compositions in the sample, wherein the biodegradable nanocluster composition are degraded by the sample after imaging. The biodegradable nanocluster composition of the imaging method of the present invention comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is below 300 nm, wherein the biodegradable nanocluster composition has an absorbance in the visible region, or an absorbance in the near infrared (NIR) range between 700 and 1200 nm, or are superparamagnetic, or have a strong magnetic relaxivity, magnetization or a combination thereof. In a specific embodiment the present invention discloses a method for treating artherosclerotic plaques in a patient comprising the steps of: (i) identifying a patient in need for treatment, (ii) administering one or more biodegradable nanocluster compositions to the sample, comprising two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is below 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof, and (ii) facilitating release of a cardiovascular drug in the body from the biodegradable optical nanocluster nanocluster upon degradation or swelling either with or without exposure to laser, high-intensity non-coherent electromagnetic irradiation, RF irradiation, or magnetic field or destroying cells that contribute to atherosclerosis by photothermolysis of the cells.

In another specific embodiment the present invention is a method for treating cancer in a patient comprising the steps of: (i) identifying one or more tumor cells or circulating tumor cells in need for treatment, (ii) administering one or more biodegradable nanocluster compositions to the sample, wherein the biodegradable nanocluster composition comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is below 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof, (iii) monitoring the uptake of the one or more biodegradable nanoclusters in the one or more tumor cells or circulating tumor cells, (iv) optionally facilitating necrosis and vaporization of the one or more tumor cells or circulating tumor cells by an exposure to laser, high-intensity non-coherent electromagnetic irradiation, RF irradiation, or magnetic field, (v) transitioning an aggressive tumor phenotype to a more benign tumor and (vi) optionally removing the one or more tumor cells or circulating tumor cells by local resection.

In yet another specific embodiment the present invention discloses a photo-thermolysis method for treating cancer and artherosclerosis by induced cell death comprising the steps of, identifying a patient in need for treatment, administering one or more biodegradable nanocluster compositions to the sample, comprising two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is below 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof, monitoring the uptake of the biodegradable nanocluster composition, and facilitating induced cell death by an exposure to laser, high-intensity non-coherent electromagnetic irradiation, RF irradiation, or magnetic field.

In another embodiment the present invention describes a method by which an active agent can be delivered to a patient in need of an active agent. The active agent as per the present invention comprises one or more biodegradable nanocluster compositions that are administered to the patient. The biodegradable nanocluster composition of the active agent comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is below 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof. The active agent is released upon biodegradation of the clusters or by heating the particles with a laser in a NIR region.

In yet another embodiment the present invention is a nanoparticle coated nanocluster composition comprising: a nanocluster composition comprising two or more nanoparticles each comprising one or more metals, metal oxides, inorganic substances, or a combination thereof, and one or more stabilizers in contact with the two or more nanoparticles to form a nanocluster composition in which an inorganic weight percentage is greater than 50% and the average size is below 300 nm, wherein the biodegradable nanocluster composition has magnetic properties, optical properties or a combination of both and a coating of one or more second nanoparticles at least partially covering the nanocluster composition.

In a separate embodiment the present invention discloses a method of making a nanorose composite of noble metal coated constituent metal oxide or magnetic nanoparticles and a stabilizer by coating a noble metal onto the surface of two or more constitute metal oxide or magnetic nanoparticles under reducing conditions in the presence of one or more stabilizers to form the nanorose composite of noble metal coated constitute nanoparticles of a metal oxide or magnetic material with a inorganic loading of greater than 50% and the average size nanorose composition is below 300 nm with wherein an absorbance in the near infrared (NIR) range between 700 and 1200 nm and magnetic properties, optical properties or a combination of both

In one embodiment the present invention is a nanorose composite comprising two or more constitute metal oxide or magnetic nanoparticles in contact with one or more stabilizers and a noble metal coating at least partially coated on the surface of two or more constitute metal oxide or magnetic nanoparticles to form the nanorose composite with an inorganic loading of greater than 50% and the average size nanorose composition is below 300 nm with an absorbance in the near infrared (NIR) range between 700 and 1200 nm, magnetic properties, optical properties or a combination thereof.

In a specific embodiment the present invention describes an imaging method for a patient in need of imaging by providing the patient with an amount of a nanorose composite comprising two or more constitute metal oxide or magnetic nanoparticles in contact with one or more stabilizers and a noble metal coating at least partially coated on the surface of two or more constitute metal oxide or magnetic nanoparticles to form the nanorose composite with an inorganic loading of greater than 50% and the average size nanorose composition is below 300 nm with an absorbance in the near infrared (NIR) range between 700 and 1200 nm, magnetic properties, optical properties or a combination thereof and imaging the patient by detection of the nanoroses.

In another specific embodiment the present invention describes a photo-thermolysis method for the treatment of cancer and atherosclerosis by necrosis or apoptosis with a NIR laser comprising the step of providing a patient in need of treatment with an amount of a nanorose composite comprising two or more constitute metal oxide or magnetic nanoparticles in contact with one or more stabilizers and a noble metal coating at least partially coated on the surface of two or more constitute metal oxide or magnetic nanoparticles to form the nanorose composite with an inorganic loading of greater than 50% and the average size nanorose composition is below 300 nm with an absorbance in the near infrared (NIR) range between 700 and 1200 nm, magnetic properties, optical properties or a combination thereof.

In another embodiment the present invention is a method for delivering an active agent comprising delivering an active agent associated with a nanorose composite comprising two or more constitute metal oxide or magnetic nanoparticles in contact with one or more stabilizers and a noble metal coating at least partially coated on the surface of two or more constitute metal oxide or magnetic nanoparticles to form the nanorose composite with an inorganic loading of greater than 50% and the average size nanorose composition is below 300 nm with an absorbance in the near infrared (NIR) range between 700 and 1200 nm, magnetic properties, optical properties or a combination thereof, whereby the drug is released upon heating the particles with a laser in the NIR region.

In yet another embodiment the present invention discloses a shaped based therapeutic nanocluster composition comprising, (i) two or more closely spaced nanoparticles each comprising one or more metals, metal oxides, inorganic substances, or a combination thereof, (ii) one or more therapeutic moieties conjugated to the two or more closely spaced nanoparticles, and (iii) one or more stabilizers in contact with the two or more nanoparticles to form a shaped based therapeutic nanocluster composition with an average size is below 200 nm, wherein the biological activity of the one or more therapeutic moieties is enhanced by the shaped based therapeutic nanocluster composition.

### DESCRIPTION OF THE DRAWINGS

For a more complete understanding of the features and advantages of the present invention, reference is now made to the detailed description of the invention along with the accompanying figures and in which:

FIG. 1 is a schematic of a biodegradable nanocluster;

FIG. 2 shows steric and electrostatic stabilization of clusters of gold mixed with PLA(2K)-PEG(10K)-PLA(2K): FIG. 2A shows the EO groups form loops in the aqueous solvent to aid stabilization; FIG. 2B shows dual electrostatic interactions of lysine ligands cross-link nanoparticles to form clusters;

FIG. 3A shows scattering spectra normalized by integrated scattering intensity of nanoclusters in cells and in solution. The spectra are normalized by their integrated intensity to compare spectral curves; FIG. 3B shows dark-field reflectance images of cells treated with nanoclusters over time (a-c) and corresponding color maps indicating wavelength at peak scattering intensity in each pixel (d-f); FIG. 3C shows Normalized scattering spectra of unlabeled cells;

FIG. 4 shows dark-field reflectance images of unlabeled cells (a-c) and corresponding maximum scattering peak color maps (d-f);

FIGS. 5, 6 and 7 are photoacoustic imaging of coated biodegradable gold nanoclusters;

FIG. 8A shows the UV-vis absorbance spectra; and FIG. 8B shows the particle size distribution for nanoclusters formed at a 1× and 2× gold loading;

FIG. 9A shows the UV-vis absorbance; and FIG. 9B shows the particle size distribution, measured by DLS, of a solution of PLA-PEG-PLA with lysine capped gold particles before the evaporation step of the nanocluster formation process;

FIG. 10A shows the UV-vis absorbance spectra of the colloidal dispersion of gold nanoparticles and the nanoclusters after varying amounts of evaporation during the formation process; FIG. 10B shows the particle size distribution, as determined by DLS of the biodegradable nanoclusters that were formed at different amounts of evaporation;

FIG. 11A shows the UV-vis absorbance spectra of gold nanoclusters after 2.5 days of incubation in pH 4, 5, and 6 media; FIG. 11B shows the UV-vis absorbance spectra of gold nanoclusters after 2.5 and 4.5 days of incubation in pH 4, 5, and 6 media;

FIG. 12A shows the dark-field reflectance image of nanoclusters immobilized on a microscope slide coated with gelatin; FIG. 12B shows the scattering spectra of 3 individual nanoclusters;

FIG. 13 is a schematic representation of a molecularly targeted plasmonic nanosensor;

FIG. 14 is a graph of the absorbance spectra of nanoclusters and solid Au sphere before and after addition of various concentrations of anti-EGFR antibody;

FIG. 15 is an image of A431 cells incubated with PEGylated nanoclusters without antibody conjugation;

FIG. 16A is an image of A431 cells incubated with nanoclusters conjugated with 25 ug/mL Ab. FIG. 16B is an close-up of image in FIG. 16A showing gold within A431 cells;

FIG. 17A shows colloidal dispersion gold nanoparticles; FIG. 17B and FIG. 17C show nanoclusters formed at pH=7.4 immediately after preparation; (D) and (E) nanoclusters formed using 1% and 0.1% alginic acid, respectively, at pH=4 after 1 week;

FIGS. 18A-18I is an electron microscopy characterization of gold nanoclusters;

FIG. 19 shows the size, shape and colloidal stability of nanorose clusters. FIG. 19A, is a SEM image on silicon wafer, including upper left inset at higher magnification, illustrate small clusters; FIGS. 19B, 19C, 19C1 and 19C2, are high resolution TEM images of a single nanorose cluster on ultra thin carbon film substrate reveals an open nanorose cluster of iron oxide and an Au primary core-shell particles. FIG. 19D shows hydrodynamic diameter in water from dynamic light scattering starts at 25 nm. FIG. 19E shows an energy dispersive spectroscopy (EDS) area scan coupled with HRTEM from one nanorose. FIG. 19F shows the magnetization vs field strength at 300K. FIG. 19G graphs an average optical density spectra vs incident light wavelength in macrophages labeled with different nanorose concentrations;

FIG. 20A is an image where the blue dispersion in the inset indicated a strong absorbance in NIR region; FIG. 20B is an image of a similar strong NIR absorbance was observed in deionized water, PBS solution and a DMEM supplemented with 10% FBS cell culture media;

FIG. 21A is an image of macrophages cultured in a DMEM supplemented with 10% FBS media without nanoroses; FIG. 21B is an image of macrophages cultured with 10 μg Au/ml nanorose in DMEM supplemented with 10% FBS media; FIG. 21C are phase contrast and dark field microscopy images of macrophages labeled with nanorose in media. The left panels do not include nanoroses. The middle and right panels at two different levels of magnification include nanoroses;

FIG. 22 shows the strong uptake of nanoroses into macrophage cells as determined by flame atomic absorption spectroscopy for 10⁵ cells;

FIG. 23 shows hyperspectral microscopy of strong absorbance at 755 nm from macrophage cells in vitro where from left to right, macrophages were incubated with nanoroses for 24 hours;

FIG. 24 shows the laser ablation of macrophage cells in vitro with a single pulse; FIG. 24A, after irradiation without nanorose, the bright field image with TUNEL staining indicates the macrophage membranes were intact; FIG. 24B, A dark field image shows interaction of the laser beam with the nanorose in the irradiated area vaporized the macrophage cells. FIG. 24C shows a temperature profile over the 2 mm diameter irradiated area;

FIG. 25 is a schematic of nanocluster of gold coated iron oxide primary particles, the lines show the gold shell domains;

FIG. 26 shows the apparatus for taking an infrared temperature measurement using HgCdTe single point detector and the temperature profile;

FIGS. 27A-27C show the nanocluster assembly platform is highly flexible and robust for controlling both the curvature of the gold shells on the primary particles and the size of the clusters and these morphologies are achieved by changing the gold to iron oxide ratio as shown;

FIG. 28 is an image of the dark field microscopy images of A431 skin cancer cells cultured with different dosage Clone 225 conjugated nanoroses;

FIG. 29 is an image of the cell uptake dosage response of clone 225 and RG16 conjugated nanoroses;

FIGS. 30A and 30C are images of the scattering spectra from hyperspectral images of cells and dark-field reflectance images FIGS. 30B, and 30D, and hyperspectral (HS) images were acquired at 24, 96, and 168 hours time points after cells were treated with nanoclusters;

FIG. 31 is an image of the specificity of nanorose uptake into peritoneal macrophages versus aortic endothelial cells and aortic smooth muscle cells by dark field microscopy with a 610 nm long pass filter;

FIG. 32 is a evidence of nanorose excretion via bile detected with 7T MRI. Due to the iron oxide core of each “nanopetal”, and the open design of the nanorose which allows a large surface area for interaction with protons (water), the nanorose have a stronger MRI signal than FDA approved FERRIDEX®;

FIG. 33 is a graph that replicate amplitude and depth measurements in rabbits measured in macrophage rich abdominal aorta, and macrophage poor thoracic aorta at up to 6 different depths;

FIG. 34A-F are TEM images of nanoclusters produced after (FIG. 34A) 0%, (FIG. 34B) 50%, (FIG. 34C) 60%, (FIG. 34D) 80%, (FIG. 34E) 100% solvent evaporation;

FIG. 35 is a schematic of lysine ligand;

FIG. 36A is an image of the particle size measurements, by DLS, and FIG. 36B is an image of the UV-vis absorbance spectra for nanoclusters composed of citrate/lysine-capped gold nanoparticles produced after different extents of evaporation;

FIG. 37 is a histogram of separation distances between primary gold nanoparticles within a nanocluster produced after 100% solvent evaporation (starting gold concentration of 3 mg/mL and a PLA-b-PEG-b-PLA/Au ratio of 16/1);

FIG. 38 is an image of the reproducibility of nanoclusters of citrate/lysine-capped gold nanoparticles in terms of (a) size and (b) optical properties. Starting gold and PLA-b-PEG-b-PLA concentrations were 3 and 50 mg/mL, respectively. Nanoclusters were produced after 100% solvent evaporation;

FIG. 39A is an image of the particle size measurements, by DLS, TEM images of nanoclusters after (FIG. 39B) 60% and (FIG. 39C) 100% solvent evaporation, and (FIG. 39D) UV-vis absorbance spectra of nanoclusters composed of citrate/lysine-capped nanoparticles assembled using PEG homopolymer (MW=3350);

FIG. 40A is an image of the particle size distribution, as measured by DLS, and FIG. 40B is an image of the UV-vis spectra of clusters of citrate/lysine-capped nanoparticles made with the mixing protocol;

FIG. 41 is an image of the UV-vis spectra of clusters of citrate-capped nanoparticles made with the mixing protocol. The starting gold concentration was 3 mg/mL and the PLA-b-PEG-b-PLA/Au ratio was 16/1;

FIG. 42 is an image of the viscosity of PLA-b-PEG-b-PLA as a function of concentration. Viscosity measurements were performed using a cone and plate viscometer;

FIG. 43 is an image of the UV-vis absorbance spectra for clusters made with gold primary particles capped with different ligands;

FIG. 44A is an image of the DLS measurements, TEM images after (FIG. 44B) 85% and (FIG. 44C) 100% solvent evaporation, respectively, and (FIG. 44D) UV-vis, absorbance spectra for nanoclusters composed of citrate-capped gold nanoparticles produced after different extents of evaporation with a starting gold concentration of 3 mg/mL and a PLA-b-PEG-b-PLA/gold ratio of 16/1;

FIG. 45 is an image of the Hydrodynamic diameter and absorbance values for nanoclusters composed of primary particles capped with citrate (▪) or a combination of citrate and lysine () ligands;

FIG. 46A is an image of the particle size distribution, as measured by DLS, and

FIG. 46B is an image of the UV-vis absorbance spectra of nanoclusters of citrate/lysine-capped nanoparticles produced with varying PLA-b-PEG-b-PLA/gold ratios at an initial gold concentration of 1 mg/mL and 100% solvent evaporation. TEM images of nanoclusters: (FIG. 46C) 16/1 polymer/gold ratio and an initial gold concentration of 3 mg/mL and (FIG. 46D) a 1/1 polymer/gold ratio with an initial gold concentration of 1 mg/mL after 100% solvent evaporation;

FIG. 47 is a TEM image and FIG. 48 is a STEM-EDS micrographs of dextran-coated iron oxide nanoparticle cluster shells on gold nanocluster cores;

FIGS. 49A and 49B are tables of gold nanocluster cores and various initial and final iron oxide to gold ratios;

FIG. 50 is a TEM image and FIG. 51 is a STEM-EDS micrographs of citrate-coated iron oxide nanoparticle cluster shells on gold nanocluster cores;

FIG. 52 is an image of the time variation of thermoleastic displacement of macrophage-rich and control rabbit aortas;

FIG. 53A is a graph of the amplitude of phase modulation vs depth for control tissue specimens; FIG. 53B is a graph of the amplitude of phase modulation vs depth for macrophage-rich tissue specimens;

FIG. 54A is an image of the replicate amplitude and depth measurements in three rabbits measured in each of two anatomical locations at up to 6 different depths. FIG. 54B is an image of the replicate amplitude and depth measurements in three rabbits measured in each of two anatomical locations at up to 6 different depths; and

FIG. 55 is a microscopy images of macrophage-rich and control tissue sections. Macrophage-rich (left column) and control tissue (right column) sections; Brightfield RAM-11 stained (top Row) and darkfield (bottom row) unstained microscopy images.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Nanotechnology can provide unique solutions to revolutionize diagnosis and treatment of many devastating diseases such as cancer. One specific area of great interest is development of nanoparticles for molecular specific imaging, therapy and combined imaging/therapy. Nanoparticles such as gold and silver with plasmonic resonances in the near-infrared (NIR) optical region, where soft tissue is the most transparent, are of great interest in the biomedical imaging. Plasmonic nanoparticles may be used for combined imaging and photothermal therapy of cancerous cells. Plasmonic nanoparticles can be combined with another inorganic material, for example iron oxide for MRI, to form hybrid nanomaterials that provide easily detectable signals in more than one imaging modality.

In addition, molecular targeted nanoparticles exhibit significantly increased avidity, and they can be simultaneously decorated with different types of biomolecules which determine their delivery, targeting specificity and molecular therapeutic properties. Therefore, plasmonic nanoparticles provide an effective solution to one of the major challenges of modern day medicine—efficient delivery of therapeutics and molecular specific treatment of pathology with real-time imaging for guidance and monitoring.

A major roadblock in translation of inorganic nanoparticles to clinical practice for systemic targeting of cancer cells is their non-biodegradable nature. In addition, sizes of coated nanoparticles that are used in biological applications are not small enough to be easily cleared from the body. The accumulation and resulting long-term toxicity of nanoparticles is a major concern. Recently, it was demonstrated that particles with hydrodynamic diameters less than 5.5 nm are efficiently eliminated from the body by urinary excretion. However, plasmonic nanoparticles with resonances in the NIR region such as gold nanoshells, nanorods and nanocages are at least 50 nm in size, and often >100 nm, severely limiting their body clearance rates.

The present invention describes the design, synthesis and characterization of biodegradable nanomaterials with enhanced contrast capabilities for non-invasive molecular imaging of cancer, and thereby eliminating the existing roadblock to clinical translation. The nanoparticles of the present invention degrade to easily clearable components in the body and, therefore, provide a crucial missing link between the enormous potential of metal nanoparticles for cancer imaging and therapy and translation into clinical practice. The synthetic methodology of the present invention is based on controlled assembly of very small (less than 5 nm) primary gold particles into nanoclusters with <100 nm overall diameter and an intense NIR absorbance. The assembly is mediated by biodegradable polymers and small capping ligands on the primary nanoparticles. The intermolecular interactions of the capping ligands and stabilizing polymer(s) is designed to control cluster growth in order to keep the primary nanoparticles in close proximity, to produce strong NIR absorbance. After delivery into the body the nanoclusters will deaggregate over time into sub-6 nm ligand capped primary gold nanoparticles, which are highly favorable for rapid clearance from the body. This hybrid polymer/inorganic material combine advantages of biodegradability of polymer nanoparticles and strong imaging contrast and therapeutic capabilities afforded by metal nanoparticles.

Properties of gold nanoparticles such as photo-stability, water dispersibility, and non-toxicity make these probes highly advantageous for biological imaging.

New opportunities in cellular optical imaging and therapy in intact tissues have been spawned by gold nanoparticles with various geometries including gold nanoshells, nanorods, and nanocages with absorbance 1000 fold those of organic dyes. For these particle geometries, the surface plasmon resonance (SPR) peak of gold shifts to the NIR region (700 to 850 nm) where tissue is the most transparent. It has been demonstrated that gold nanoparticles provide high contrast in imaging of cancerous cells using confocal reflectance microscopy, dark-field imaging, two-photon luminescence, phase-sensitive OCT, and photoacoustic imaging. The latter imaging modality is particularly relevant to cancer imaging as its penetration depth is superior as compared to other optical imaging methods.

Plasmonic gold nanoparticles can function both as delivery vehicles and as contrast agents that enhance photothermal therapy when they absorb near infrared (NIR) irradiation. Photothermal therapy has been demonstrated using NIR absorbing nanoshells and nanorods or through the use of molecular-targeting spherical nanoparticles which undergo molecular specific aggregation that results in red- to NIR-shifted resonances due to plasmon coupling. In addition it is possible to use either pulsed or CW irradiation to achieve cell killing while the mechanism of cell death might be different in either case the end result is the same.

The synthesis of hybrid multimodal nanoparticles combine useful properties of more than one nonmaterial like gold-coated iron oxides for combined optical/MRI imaging and therapy was demonstrated. The nanoparticles have a magnetic core which provides strong T₂-weighted contrast, while the gold shell can be tuned to absorb in the infrared. These type of nanoparticles have been also used for molecular-specific optical image contrast enhancement using magnetic modulation.

The biodegradable nanoclusters comprises 3, 5, 10, 20, 25, 50, 100, 1000, 2000, and so on up to 1,000,000 or more primary nanoparticles. The biodegradable nanocluster of the present invention has an average size of about 3, 5, 10, 20, 25, 50, 60, 70, 80, 90, 100, 125, 150, 175, 200, 250 and 300 nm and the stabilizer to the primary nanoparticle weight ratio is about 10%, 15%, 20%, 25%, 30%, 35%, 40%, 45% and 50%. The biodegradable nanocluster described in the present invention deaggregates into one or more particles; wherein said particles have an average size of less than 15 nm, in vitro, in vivo, a biologically relevant media, a cell culture, in a human subject, and in an animal subject over a period of one-few hours, 1 day, 2 days, 3 days, 4 days, 5 days, 6 days, 1 week, 2 weeks, 5 weeks and 10 weeks or more.

The saturation magnetization of a dried biodegradable nanocluster particle dispersion at 300 K is above 30 emu/g iron oxide when measured by a superconducting quantum interference device. The primary nanoparticles of the biodegradable nanocluster of the present invention are magnetic and comprise a spin-spin relaxivity (reciprocal of the spin-spin relaxation time T2) sufficiently large to provide enhanced contrast in a MRI image. The invention further describe increasing the spin-spin relaxivity by: (i) increasing the number of primary magnetic nanoparticles within the cluster; wherein the number of primary magnetic nanoparticles is greater than 5, 10, 20, 30, 40 or 50, 100, 1000, 2000, and so on up to 1,000,000 or more primary nanoparticles and (ii) raising a volume fraction of a magnetic material within the cluster; wherein the volume fraction of magnetic material is greater than 0.1, 0.2, 0.3, 0.4, 0.5 or 0.6.

In a certain aspect of the present invention the biodegradable nanoclusters have magnetic properties, optical or electromagnetic properties or a combination of both, and the metal oxide particles are at least partially magnetic. In another aspect the metals used in the primary nanoparticles can comprises Fe, Ni, Co, FePt, or alloys of these materials and have a general formula MFe₂O₄ where M=Mn, Fe, Co, Ni. The size of a metal core in the primary nanoparticles is 2 nm, 3 nm, 5 nm, 10 nm, or 20 nm. The one or more primary metal oxides are selected from iron, cobalt, magnesium, zinc, aluminum oxides or combinations thereof.

In yet another aspect of the present invention the one or more stabilizers comprise a biocompatible polymer, a biodegradable polymer, a multifunctional linker to form a liposome without the use of a surfactant, or combinations thereof. In a further aspect the biodegradable nanocluster comprises one or more therapeutic moieties, one or more non-therapeutic moieties or a combination to target cancer. The therapeutic moieties associated with the biodegradable nanoclusters of the present invention include folic acid, peptides, proteins, antibodies, siRNA, poorly water-soluble drugs, anti cancer drugs or combinations thereof.

The invention further describes the distribution of the primary nanoparticles. The nanoparticles are distributed throughout the cross section of the total particle and not just near the surface, 10%, 20%, 30%, 40%, 50%, 60%, 70%, 80% or 90% of the primary particles are not in the outer 25% of the radius of the biodegradable nanocluster. The biodegradable nanoclusters of the present invention are stable during storage. The present invention also provides a method of biodegradation of the biodegradable nanocluster by changing the pH, a NIR light, a visible light, applying a magnetic or electrodynamic field (the latter includes RF and microwave), an enzymatic or chemical addition, or a combination of the above methods. The biodegradable nanoclusters of the present invention have an absorbance in the near infrared (NIR) range between 700 and 900 nm with a cross section of at least 10⁻³, preferably 0.02 cm²/microgram of metal for a metal concentration in the dispersion in the range of 0.5 to 3.0 mg/mL. The biodegradable nanoclusters deaggregate over time into one or more primary particles in vitro or in vivo; wherein the one or more primary particles have an average metal size of 5 nm or lower and an average hydrodynamic diameter of 15 nm or lower.

The invention further describes the stabilizers that are used in the formation of the biodegradable nanoclusters, these include one or more stabilizers are further defined as one or more primary particle stabilizers, one or more secondary stabilizers, or both, selected from starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone) or combinations thereof.

In one aspect of the present invention the size and proximity of the metal nanoparticles and the overall biodegradable nanocluster size is controlled to maximize absorbance in the NIR, or radio-frequency (RF) loss tangent, or T2 relaxation time. In another aspect the one or more ligands on the metal nanoparticles facilitate renal clearance, liver clearance, intestinal clearance or combinations thereof.

In one embodiment the present invention describes a biodegradable nanocluster composition with an average size below 150 nm comprising: one or more primary metal nanoparticles; one or more stabilizers; wherein said stabilizer to metal nanoparticle weight ratio is less than 50%; and one or more pharmaceutically acceptable carrier; wherein the biodegradable nanocluster has an absorbance in a near infra-red window between 700 nm and 850 nm.

In one aspect the biodegradable nanocluster have an absorbance in the near infrared (NIR) range between 700 and 900 nm with a cross section of at least 10⁻³, preferably 0.02 cm²/microgram of metal for a metal concentration in the dispersion in the range of 0.5 to 3.0 mg/mL. In another aspect the absorbance of the biodegradable nanocluster at 750 nm is greater than absorbance of the biodegradable nanocluster at 550 nm; the absorbance of the biodegradable nanocluster at 750 nm is at least one-half of absorbance of the biodegradable nanocluster at 550 nm; the absorbance of the biodegradable nanocluster at 750 nm is 40%, 30%, 20% of the absorbance of the biodegradable nanocluster at 550 nm

In an another embodiment the present invention describes a medical biodegradable nanocluster composition comprising, one or more primary metal oxides or magnetic nanoparticles; one or more noble metals at least partially coating the primary metal oxides or magnetic nanoparticles; one or more stabilizers; one or more active ingredients; and one or more biodegradable polymers dispersed in or about the coated nanoparticles; wherein the coated nanoparticles have an average size of less than 120 nm.

In one aspect of the present invention the one or more noble metals are at least partially coated onto the surface of the primary metal oxides or magnetic nanoparticles under reducing conditions in the presence of the one or more stabilizers. In another aspect the one or more stabilizers comprise a biocompatible polymer. In yet another aspect the biodegradable nanoclusters have absorbance in the near infrared (NIR) range between 700 and 850 nm and in the visible region. In a further aspect the biodegradable nanoclusters deaggregate in vivo or in vitro over time into one or more particles; wherein the one or more particles have an average size of 5 nm or lower.

The one or more primary metal or metal oxides are selected from gold, iron, magnesium, zinc, aluminum oxides, silicon oxides or combinations thereof, and the one or more noble metals partially coated onto the surface of the primary metal oxides are selected from silver, gold, copper, platinum, palladium, iridium, rhodium or combinations and alloys thereof. The one or more biodegradable polymers used in the present invention are selected from, PEG, dextran, polyvinyl alcohol, polyvinylpyrollidone, polyacrylates, polymethacrylates, polyacrylic and polyacrylamide-based gels or polymers, poly(vinyl alcohol), polypeptide hydrogels, poly(methacrylic acid), poly(vinylpyrrolidone), co-copolymers, poly (β-hydroxybutyrate) diol, poly (lactic acid) diols, polyglycolide diols, polylactide diol, polycaprolactone diol, polyglycolic acid diol polyanhydrides, polypeptides, albumin, alginates, amino acids, thiols, amines and carboxylic acids or combinations thereof. The one or more active ingredients are enclosed with the one or more biodegradable polymer matrices comprise one or more of drugs, proteins, amino acids, peptides, medical imaging agents, or combinations thereof.

The one or more drugs that can be used in the biodegradable nanoclusters of the present invention are selected from antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or combinations thereof. The one or more imaging techniques that can be used in conjunction with the biodegradable nanoclusters of the present invention include optical coherence tomography (OCT), photoacoustic, ultrasonic, fluorescence, medical diagnostic, magnetic resonance imaging, photothermal imaging or combinations thereof.

The present invention is also a method for imaging a patient comprising the steps of: identifying a patient in need of imaging; administering one or more biodegradable nanocluster compositions comprising an imaging agent dispersed in a suitable aqueous or non-aqueous medium, wherein the biodegradable nanoclusters are superparamagnetic and have an absorbance in the visible region, an absorbance in the near infrared (NIR) range between 700 and 850 nm; facilitating degradation of the biodegradable nanoclusters by one or more external agents; releasing of the imaging agent in the body; and imaging the patient by detection of the nanoclusters. In a certain aspect the imaging described in the present invention is a magnetic resonance imaging, an optical imaging, both magnetic and optical imaging, an optical coherence tomography, a photoacoustic tomography, an ultrasound imaging a magnetomotive ultrasound imaging and a hyperspectral microscopy. The biodegradable nanocluster composition of the present invention is administered subcutaneously, intraveously, peritoneally, orally, intramuscularly, topically, nasally, intradermally, ocularly, rectally, vaginally or combinations thereof. In a further aspect the external agents for the degradation of the biodegradable nanocluster and release of the imaging agent comprise magnetic fields, ultrasound techniques, laser or high intensity optical heating, magnetic, optical disruption or combinations thereof. 40%, 50%, 60%, 70%, 80% or 90% of the metals from the biodegradable nanocluster of the present invention clears from the body within 1 day, 1 week, 1 month and 2 months. 99% of the metals from the biodegradable nanocluster of the present invention clears from the body within 1 day, 1 week, 1 month and 2 months. In yet another embodiment, the present invention also provides a method of treating cancer and can include imaging with photothermolysis, or imaging with drug delivery, or combination of thereof.

In a further embodiment the present invention is a method for treating macrophage induced angiogenesis in a cancer patient comprising the steps of: identifying a patient in need for treatment; administering one or more biodegradable nanoclusters containing one or more anti-cancer agents dispersed in a suitable aqueous or non-aqueous medium intravenously; wherein the biodegradable nanoclusters have an absorbance in the in the visible region and in the near infrared (NIR) range between 700 and 850 nm; monitoring the uptake of the one or more biodegradable nanoclusters in the one or more tumor-associated macrophages (TAM); facilitating necrosis and vaporization of the TAM by a laser exposure; transitioning an aggressive tumor phenotype to a more benign tumor; and removing the benign tumor by local resection.

The present invention also describes a photo-thermolysis method for treating cancer and atherosclerosis by induced cell death comprising the steps of: identifying a patient in need for treatment; administering a biodegradable nanoclusters composition; wherein the biodegradable nanocluster composition is superparamagnetic and has an absorbance in the visible region, an absorbance in the near infrared (NIR) range between 700 and 850 nm; monitoring the uptake of the biodegradable nanocluster composition; and facilitating induced cell death by a laser or high-intensity optical exposure. The photothermolysis as described in the present invention occurs within a cell.

The present invention also provides a method for delivering an active agent comprising the steps of: identifying a patient in need of the active agent; administering the active agent; wherein the active agent is associated with a biodegradable nanocluster comprising a primary metal primary particle or a metal oxide primary particle and a polymeric stabilizer; and releasing the active agent by heating the particles with a laser or other optical source in a NIR region. In certain aspects the biodegradable nanocluster comprises a hydrodynamic diameter smaller than 100 nm and has an absorbance in the NIR window between 700 nm and 850 nm corresponding to at least 10⁻³, preferably 0.02 cm²/microgram of metal for a metal concentration in the dispersion in the range of 0.5 to 3.0 mg/mL.

The present invention also describes a method forming a nanocluster comprising the steps of: forming an aqueous dispersion; wherein the aqueous dispersion comprises one or more primary particles and one or more dispersed or dissolved stabilizers; and aggregating the one or more primary particles and the one or more dispersed or dissolved stabilizers of the aqueous dispersion over time to form the nanocluster. The nanocluster formation is aided by evaporation of 20%, 50%, 70% and 90% of a solvent and the nanoclusters are recovered by adding an aqueous solution which may comprise a stabilizer; wherein said stabilizer includes polyvinyl alcohol, polyethylene glycol, polysaccharides, and nonionic surfactants.

In a certain embodiment, the present invention is a method of forming a biodegradable gold nanocluster by a double emulsion tem plating process comprising the steps of: dispersing one or more gold nanoclusters stabilized by a legend in an aqueous medium to form an inner water phase; dissolving one or more polymers in an organic solvent to form an organic phase; dissolving a natural biodegradable polymer in an aqueous medium to form the outer water phase; mixing the inner water phase, the organic phase and the outer water phase to form a mixture; and emulsifying the mixture to form the biodegradable gold nanoclusters. In one aspect the ligand comprises lysine, and other amino acids, proteins, and peptides. In another aspect the natural biodegradable polymer comprises alginic acid. In yet another aspect the polymers in the organic phase comprise, PLA, PEG, and other natural and synthetic biodegradable polymers.

Intravenous administration is the most effective method for delivery of imaging and therapeutic agents because blood stream very quickly distributes the administered agent throughout the body. Eventually nanoparticles are cleared from blood by the reticuloendothelial system (RES) and kidneys. Generally, particles larger than 200 nm are cleared by the spleen, while nanoparticles smaller than 100 nm are mainly cleared by the liver, and nanoparticles with hydrodynamic size smaller than about 5.5 nm undergo effective renal clearance. Qdots with zwitterionic (cysteine) and neutral (PEG) coatings were cleared the most efficiently. However, coated 5 nm gold nanoparticles with positive surface charge showed better excretion in urine and feces than negatively or neutral counterparts.

The shapes and compositions of nanoparticles may be guided during condensation of atoms by selectively favoring growth of particular crystal facets to produce spheres, rods, wires, discs, cages, core-shell structures and many other shapes. Typically gold particles with these shapes with a size on the order of 100 nm would be inert and thus not biodegrade into sub-10 nm gold entities that would be desirable for facilitating clearance.

A less common yet highly adaptable approach is to assemble ultrasmall nanoparticles (<10 nm) as the primary building blocks, rather than atoms, into 1D 2D and 3D inorganic/organic nanocluster composites. The size and shape of 3D composite nanoclusters have been controlled with block copolymer templates, DNA, proteins and viruses, primarily for the design of sensing and memory devices. In nearly all cases, these nanoclusters grow to sizes well above 100 nm. Recently, gold particles were grown on the surface of liposomes. This reaction produced NIR absorbing gold nanoshells, which can be degraded by a surfactant to small (<10 nm) gold/phospholipid complexes.

The present invention describes a design for a hybrid polymer/inorganic nanoclusters smaller than ˜100 nm with high levels of targeting/imaging/therapy functionality. These nanoclusters consist of individual primary particles coated with small capping ligands. The cluster morphology will be controlled by the intermolecular interactions of the capping ligands and by biodegradable templating polymers. These nanoclusters biodegrade back into individual primary particles in the body that will facilitate their excretion. The clearance, excretion rates and pathways can be predetermined by the size of the primary nanoparticles and physiochemical properties of capping ligands and templating polymers. This approach provides a flexible platform for designing and validation of various types of nanoparticles for safe clinical use. Different types of primary nanomaterials can be clustered together providing multiplexing opportunities for synthesis of multifunctional/multimodal nanoparticles. Further applications include drug encapsulation inside the nanoclusters with controlled release that can be triggered by one of the following stimulus: polymer degradation in tumor microenvironment, enzyme sensitive polymers, or by an external stimulus such as NIR light or magnetic fields.

The present invention describes development of biodegradable plasmonic nanoclusters with strong absorbance in the NIR region required for effective application to in vivo optical contrast enhancement and photo-thermal therapy. In order to produce a significant red-shift, strong inter-particle coupling is required, and therefore constituent particles must be closely-spaced. Further, the magnitude of the inter-particle coupling also increases with the number of neighboring particles. Therefore, the degree of red-shift can be controlled by controlling inter-particle spacing and by modifying the particle volume-packing arrangement. The present invention describes nanoclusters which are ideally suited for in vivo molecular imaging and photothermal therapy with plasmonic nanoparticles.

In the near infrared optical region, plasmonic nanoparticles absorb light strongly (on the order of tens of inverse centimeters) while background absorption is only about 0.03-0.05 cm⁻¹ in tissue. Therefore, a technique for in vivo, depth-resolved measurement of optical absorption properties would be an optimal method to assess the presence and distribution of plasmonic nanoparticles in tissue. Such technique named photo/opto/thermo-acoustic imaging exists, and aims to remotely estimate optical properties of tissue and plasmonic nanoparticles at high spatial and temporal resolution.

Specifically, during photoacoustic imaging the tissue is irradiated with short (5-10 ns) pulses of low energy laser light. The 15-20 mJ/cm² laser fluence of near-infrared irradiation will be sufficient to deliver optical energy to the plasmonic nanoparticles and adjacent tissue—this laser fluence is well within the safe level of laser irradiation of tissue defined by the American National Standards and FDA. Therefore, a photoacoustic level of pulsed laser energy will not produce any thermal damage to the tissue, and will result in a negligible temperature increase. Next, through the processes of optical absorption followed by thermoelastic expansion, broadband acoustic waves are generated within the irradiated volume. Using an ultrasound detector, these waves can be detected and spatially resolved. The received acoustic signal contains information about both position (time of flight) and strength of the optical absorber (amplitude of the signal). The amplitude of the thermoelastic response of the tissue is proportional to the optical absorption, i.e., the stronger the absorption, the stronger the signal. Therefore, contrast in photoacoustic imaging is primarily determined by optical contrast of tissue constituents. Furthermore, the contrast mechanism in photoacoustic imaging offers the prospect of identifying functional properties of nanoparticles at sufficient depth in tissue such properties are indistinguishable using other imaging modalities such as ultrasound, MRI, PET or CT/X-ray.

The measurements of optical properties of tissues are limited, quite variable but they can offer an approximate guide to the optical behavior of tissues. In the near-infrared (2000-3000 nm) region, water is the dominant absorber; the light penetration depth (the distance through tissue over which diffuse light decreases in fluence rate to 1/e or 37% of its initial value) varies from about 1 mm to 0.1 mm. At the other end of the spectrum, in the ultraviolet region near 300 nm, the absorption depth is shallow, owing to absorption by cellular macromolecules. In the central region, tissue absorption is modest while contrast between tissue components remains high. Within 600-900 nm wavelength, background absorption of tissue is only about 0.03-0.05 cm⁻¹ and the average optical penetration depth is on the order of tens of millimeters while the plasmonic nanoparticles absorb light strongly (on the order of tens of inverse centimeters). Therefore, the 600-900 nm spectral range is very suitable for photoacoustic imaging of plasmonic nanoparticles.

Furthermore, the photoacoustic imaging was augmented by ultrasound imaging these imaging systems are complementary. Indeed, photoacoustic imaging can be transparently integrated with ultrasound since both photoacoustic and ultrasound imaging systems utilize the same ultrasound sensor and associated receiver electronics. The ultrasound imaging visualizes the overall anatomical features of tissue while the photoacoustic imaging will identify the presence, location and functional state of the plasmonic nanoparticles.

The present invention uses combined ultrasound and photoacoustic imaging because of several major factors. First, ultrasound and photoacoustic imaging are complementary. Second, nanoparticles can be imaged within the anatomical (morphology) and even functional (activity) properties of the surrounding tissue using ultrasound-guided photoacoustic imaging. Third, ultrasonic and optical access to zenographic models of cancer is very good since the tumor is typically located within a few centimeters from the transducer. High frequency, and hence high spatial resolution, ultrasound and photoacoustic imaging is possible in most cases. Fourth, both ultrasound and photoacoustic imaging methods are a non-ionizing imaging method and there are minimal safety concerns associated with low-fluence, non-ionizing laser irradiation. In addition, ultrasound-guided photoacoustic imaging is relatively inexpensive and portable. Finally, no other imaging technique is capable of imaging functional state of nanoparticles in vivo and at sufficient (15-20 mm) depth.

The present invention describes the development of contrast agents based on metal nanoparticles for imaging of epidermal growth factor receptor (EGFR), metallo-proteases 2 and 9, oncoproteins associated with HPV 16 induced carcinogenesis, and actin. Non-linear phenomena is exhibited by nanoclusters of plasmonic nanoparticles. Biologically active agents may be added to the nanoparticles for molecular specific optoacoustic imaging of cancer cells and for selective detection of macrophages in biological models of atherosclerotic plaques. Bi-modal MRI/optical nanoparticles for combined MRI/optical molecular imaging and photothermal treatment of cancer have been demonstrated. Multimodal nanoparticles offer exciting opportunities for new strategies for combined detection, diagnosis, treatment and monitoring of carcinogenesis in future clinical practice. Sokolov et al. have also reported the first multi-functional imaging platform using plasmonic nanoparticles that incorporates both cytosolic delivery and targeting moieties on the same entity for imaging of intracellular targets such as actin.

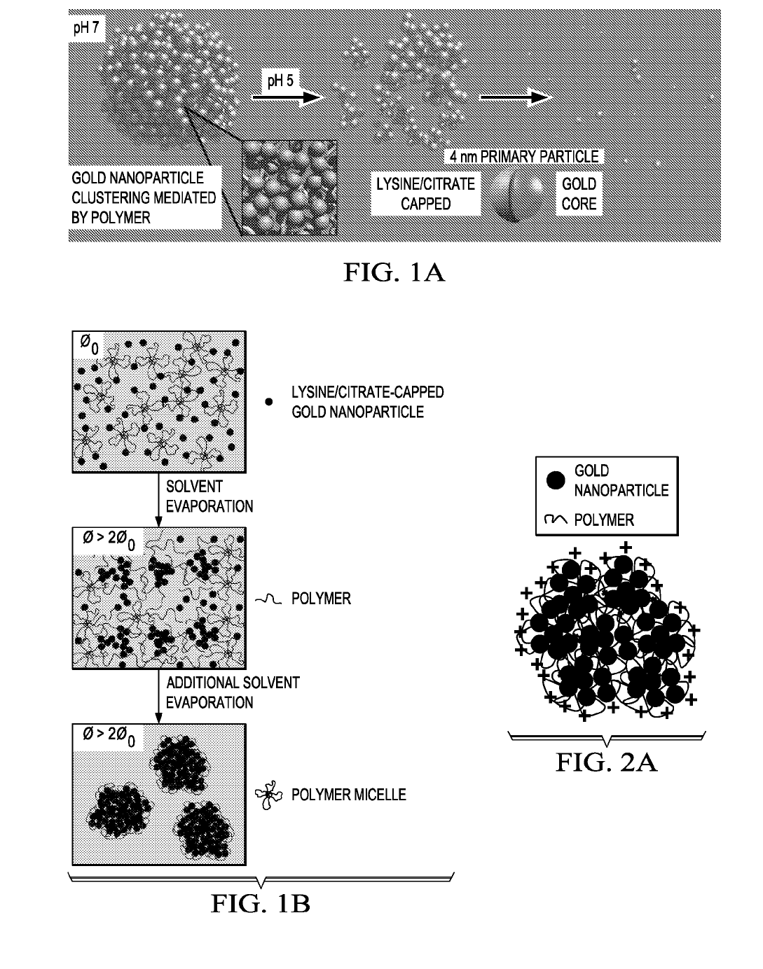

FIG. 1 is a schematic of a biodegradable nanocluster. FIG. 1A is an illustration of a biodegradable nanocluster, which is composed of ˜4-nm primary gold particles held together with a biodegradable polymer. Upon polymer degradation, catalyzed by low pH in endosomal compartments of cells, the nanocluster deaggregates into primary gold nanoparticles. FIG. 1A is a schematic of nanocluster formation process, in which primary gold nanoparticles aggregate in the presence of a polymer in a controlled manner to yield sub-100 nm clusters. Polymer adsorption to the nanoparticle surface and an increase in the volume fraction of particles, φ, via solvent evaporation promotes cluster formation. Long PEG loops on the polymer extend into the aqueous environment and provide steric stabilization for clusters.

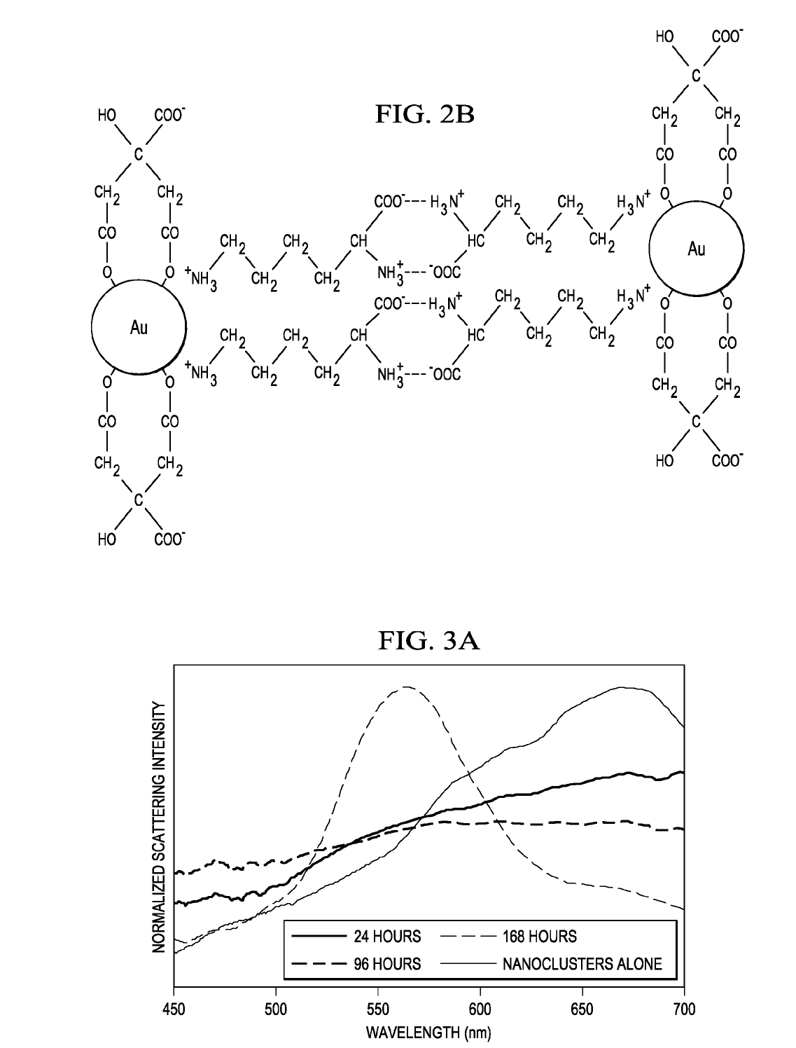

Lysine capped gold particles were found to form clusters in the presence of a biodegradable tri-block copolymer of lactic acid (LA) and ethylene glycol (EG), PLA(2K)-PEG(10K)-PLA(2K) upon concentration by solvent evaporation and resuspension. The intense NIR absorbance was produced by the close proximity of the gold primary particles resulting from electrostatic cross-linking interactions between the lysine ligands. (FIG. 2). Furthermore, the combination of the lysine ligands and PLA-PEG-PLA templating polymer provided controlled cluster growth such that the final average cluster size was smaller than 100 nm. Clusters of lysine-coated gold particles formed without polymer present grew to undesirable sizes about 10 fold larger as reported previously. Thus, the polymer is required to mediate the cross-linking of the ligands then to provide overall steric stabilization for the nanoclusters.

Gold nanoparticles (4-nm) stabilized with citrate ligands were synthesized based on a well known method from 1% HAuCL4.3H₂O. To replace the citrate ligands on the gold nanoparticles with lysine, a 1% lysine in pH 8.4 phosphate buffer (10 mM) solution was added to a 3.0 mg/mL colloidal gold solution to yield a final lysine concentration of 0.4 mg/mL. It was stirred for 2 hours. The biodegradable polymer, PLA(2K)-PEG(10K)-PLA(2K) (Sigma Aldrich, St. Louis, Mo.) was added to the aqueous dispersion of lysine capped gold nanoparticles and sonicated in a bath sonicator for 30 minutes. The polymer/gold dispersion was placed under an air stream and dried to completion over ˜2 hours. The initial lysine capped 4±0.8 nm gold particles changed in color from ruby red to blue in the presence of the tri-block copolymer, PLA(2K)-PEG(10K)-PLA(2K) during solvent evaporation (FIG. 7). Complete evaporation produced a smooth blue film, providing a preliminary indication that the surface plasmon absorbance shifted to the red-NIR region that was confirmed using UV-Vis-NIR spectroscopy. The dried film was redispersed with DI water to yield a blue dispersion.

The nanocluster morphology was observed by scanning electron (SEM) and transmission electron microscopy (TEM). A Zeiss Supra 40VP field emission SEM was operated at an accelerating voltage of 5-10 kV. The samples were prepared by depositing a dilute aqueous dispersion of the nanoclusters onto a silicon wafer. The sample was dried and washed with DI water to remove excess polymer. TEM was performed on a FEI TECNAI G2 F20 X-TWIN TEM using a high-angle annular dark field detector and an accelerating voltage of 80 kV. High resolution transmission electron microscopy (HRTEM) was performed on a TECNAI G2 F20 X-TWIN microscope in both bright field and scanning transmission electron microscopy (STEM) mode at an accelerating voltage of 200 kV. Energy dispersive x-ray elemental analysis (EDX) mapping was acquired with a dwell time of 3000 ms at any given position, and the map size was 400 positions per nanostructure. Nanocomposites were deposited from a dilute aqueous dispersion onto 200 mesh carbon-coated copper TEM grids. UV-vis spectra were obtained with a Varian Cary 5000 spectrophotometer and a 1 cm path length. Dynamic light scattering (DLS) measurements of hydrodynamic diameter were performed in triplicate on a custom-built apparatus (scattering angle: 90°) and the data were analyzed using a digital autocorrelator with a non-negative least-squares (NNLS) method. The dispersion concentration was adjusted with DI water to give a measured count rate between 300-400 kcps. All dispersions were filtered through a 0.1 μm PVDF (Millipore, Cork, Ireland) or 0.2 μm cellulose acetate filter and probe-sonicated for 2 min prior to measurement.

Upon redispersion in ˜10 ml of DI water, the hydrodynamic diameter of ˜77% by volume of the nanoclusters ranged from 60-90 nm as determined by dynamic light scattering (DLS). The primary 4-nm gold nanoparticles were uniformly dispersed throughout the sub-100 nm nanoclusters as determined by the red color in elemental analysis using STEM-EDX. A higher density of gold is seen towards the center of the cluster compared to the edges, which are somewhat enriched by the polymer coating during evaporation. The nanoclusters were surrounded by a thin polymer shell. The PEG blocks of the polymer, extend into the aqueous environment to provide effective steric stabilization of the nanoclusters.

The ruby red initial 4 nm gold nanocrystals exhibited the well-known maximum at 520 nm. However, the color change to blue with solvent evaporation indicated the formation of the gold clusters. Upon redispersion into 10 mL of DI water (10 fold the volume of water prior to solvent evaporation), the nanoclusters were stable and did not deaggregate based on the size from DLS and TEM, and the absorbance spectra. The strong NIR absorbance of the nanoclusters is expected given close spacing of nanoparticles within this nanomaterial. The extinction coefficient at the maximum absorbance, ∈₇₀₀, was calculated to be 0.020 cm²/μg, comparable to the value for nanoshells, nanocages, and nanorods.

The protonated amino end groups on the lysine readily adsorb to the gold nanoparticle surfaces in pH 7.4 media. A pair of electrostatic interactions between protonated amino groups and carboxylate couples (crosslinks) the nanocrystals. Without polymer, the cluster growth was found to be excessive with a color change to blue, as reported previously, even without any solvent evaporation. With the addition of PLA-PEG-PLA (50 mg/mL), the color changed only modestly over 4 hours indicating limited nanoparticle assembly. These results suggest that the cross-linking interactions between the NH3⁺ and COO⁻ were mediated by competing interactions with the ether oxygens on the polymer. Even with polymer present, the gold particles were close enough together to give the strong NIR absorbance, unlike the behavior of most previous clusters. The interparticle distance within the nanocluster was estimated to be 1.60 nm, based on the more discernible peripheral particles. The theoretical length of a lysine-lysine dipeptide is 1.49 nm. Thus, the short length of the lysine ligands, as well as electrostatic interactions, promote tight packing of the gold particles needed for NIR absorbance. Furthermore, the polymer was required to mediate the cross-linking of the ligands to provide overall steric stabilization for the nanoclusters.

Nanocluster size was investigated upon degradation of the PLA-PEG-PLA at pH 7.4 and 4; where pH 7.4 models normal cellular and extracellular environments and pH 4 is about 1 pH unit below that in cellular lysosomes. After storage for 1 week in pH 7.4 buffer, the more prevalent peak measured by DLS shifted modestly to smaller sizes and became broader, whereas the less prevalent peak at larger sizes became much smaller. The half life of PLA (MW=2K) is about 4 weeks at pH 7, thus only partial degradation was present, consistent with the relatively small change in hydrodynamic diameter distribution. Thus, the deaggregation was very limited. The addition of HCl to lower the pH to 4 accelerated hydrolysis of the PLA resulting in a marked shift in the hydrodynamic diameter after one week, with 72% of the particles by volume ranging in sizes from 7 nm to 11 nm.

The above results were confirmed by analysis of 100 particles in by TEM. The size was 4.3±1 nm and the previously observed clusters were no longer present. The TEM size of the gold cores was smaller than the hydrodynamic radius as expected. The ligand thickness of approximately (9−5)/2=2 nm was fairly consistent (a little longer) with the length of the lysine ligands. The degradation of the biodegradable nanoclusters was further observed visually, with a change in the color of the gold dispersion from blue in the clustered state to pink in the deaggregated state. This color transition was quantified by a notable shift in the maximum of the extinction spectrum to ca. 531 nm, close to the value of the initial spherical gold nanoparticle. This characterization of the particle morphology and the extinction spectra indicate nearly complete deaggregation of the gold nanoclusters. In a control experiment without added block copolymer, the lysine coated gold particles were found to deaggregate partially near pH 4, indicating a weakening of the cross-linking interactions upon COO⁻ protonation.

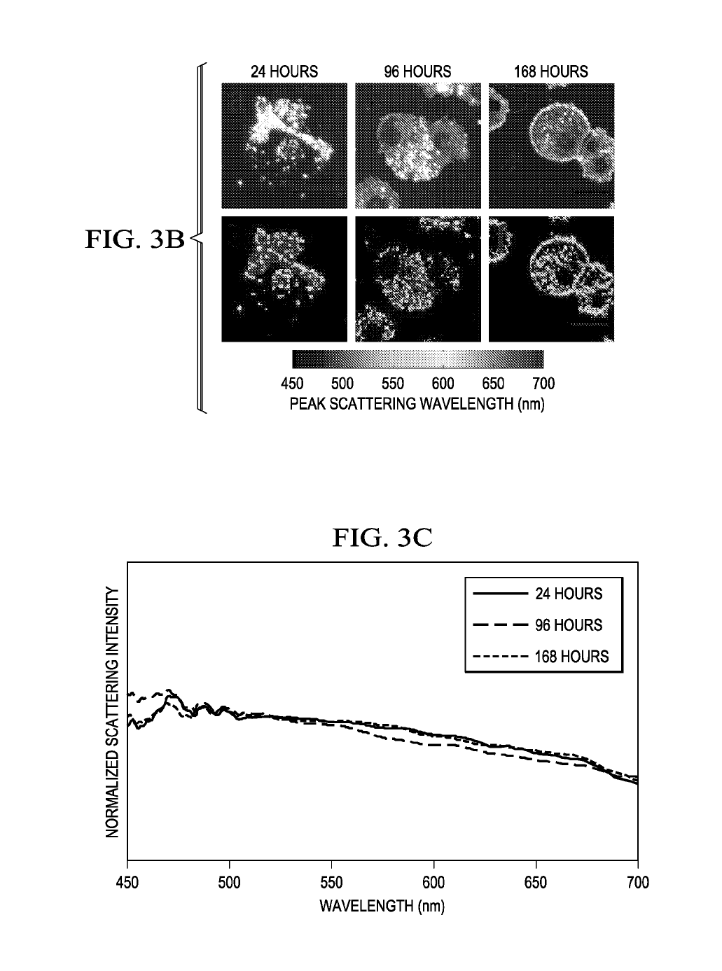

For analyzing the interactions of nanoclusters inside living cells, murine macrophage J774A.1 cells were allowed to interact with biodegradable nanoclusters for 2 hours, then the excess of nanoclusters was washed and cells were grown in phenol-free DMEM medium supplemented with 5% fetal bovine serum. Untreated cells were used as control. Dark-field reflectance (DR) images (FIG. 3B and FIG. 4, a-c), hyperspectral images (FIG. 3B and FIG. 4, d-f), and hyperspectral scattering spectra (FIGS. 3A and 3C) were acquired at time points 24, 96, and 168 hours to characterize changes in morphology of the nanoclusters in cells. High uptake of nanoclusters is evident in DR images of macrophages where nanoclusters strongly scatter illumination light (FIG. 3B, a); the scattering intensity decreases over time as macrophages divide and nanoclusters are distributed between daughter cells (FIG. 3B, a-c). Analysis of scattering from labeled cells shows significant increase of signal in the red-NIR region as compared to unlabeled cells (compare FIGS. 3A and 3C). This increase is consistent with high scattering efficiency of nanoclusters in solution (FIG. 3A). The combination of scattering from nanoclusters inside cells and intrinsic cellular scattering (FIG. 3C) produces the spectral profile of labeled cells that is shown in FIG. 3A. The relative intensity of red-NIR scattering signal decreases at 96 hours time point and eventually the scattering from labeled cells shows a marked blue shift to ca. 550 nm that is consistent with scattering from primary gold nanoparticles. These optical changes can be also followed by hyperspectral imaging (FIG. 3B, d-f); the images are color-coded according to the scattering peak position at each pixel in the field of view. A gradual progression can be observed from very strong scattering around 700 nm to 650 nm and, finally, 500-550 nm region (FIG. 3B, d-f). The expected scattering for the control macrophages gradually increases with a decrease in wavelength and does not significantly change with time. The results in cells are consistent with optical changes of nanoclusters that were observed during degradation of nanoclusters in solution. The cell assays indicate that nanoclusters biodegrade in cellular environment most likely inside lysosomes. We are carrying out TEM studies of labeled cells to determine the location and morphology of the biodegradation products.

The contrast mechanism in deep-penetrating photoacoustic imaging is based upon the difference in optical properties of the tissue constituents and contrast agent. Gold nanoparticles have excellent biocompatibility and the conjugation protocols to attach proteins to gold nanoparticles are also well developed. Even more, the photoacoustic imaging with gold nanoparticles can be potentially extended to a combined diagnostic imaging and therapy approach. Based on the information obtained with photoacoustic imaging, pulsed or continuous wave photothermal therapy could be performed to induce localized destruction of tumor, potentially even using the same light source as was used in photoacoustic imaging (PA).

PA imaging can be used to monitor changes in optical properties of gold nanoparticles in vivo. For these studies, a Cortex ultrasound imaging system (Winprobe Corporation, North Palm Beach, Fla., USA) with an integrated imaging probe was used to obtain combined ultrasound and photoacoustic images. The integrated imaging probe consisted of a 7.5 MHz center frequency transducer (14 mm wide, and 128 element linear array) and a fiber bundle for laser light delivery. Either a Q-switched Nd:YAG laser (532 nm wavelength, 5 ns pulses, 20 Hz pulse repetition frequency) or a tunable OPO laser system (680 nm-950 nm wavelength, 7 ns pulses, 10 Hz pulse repetition frequency) were used to generate photoacoustic transients. The light delivery and RF acquisition together made up the PAUS system which could capture spatially co-registered RF data from both ultrasound and photoacoustic imaging.

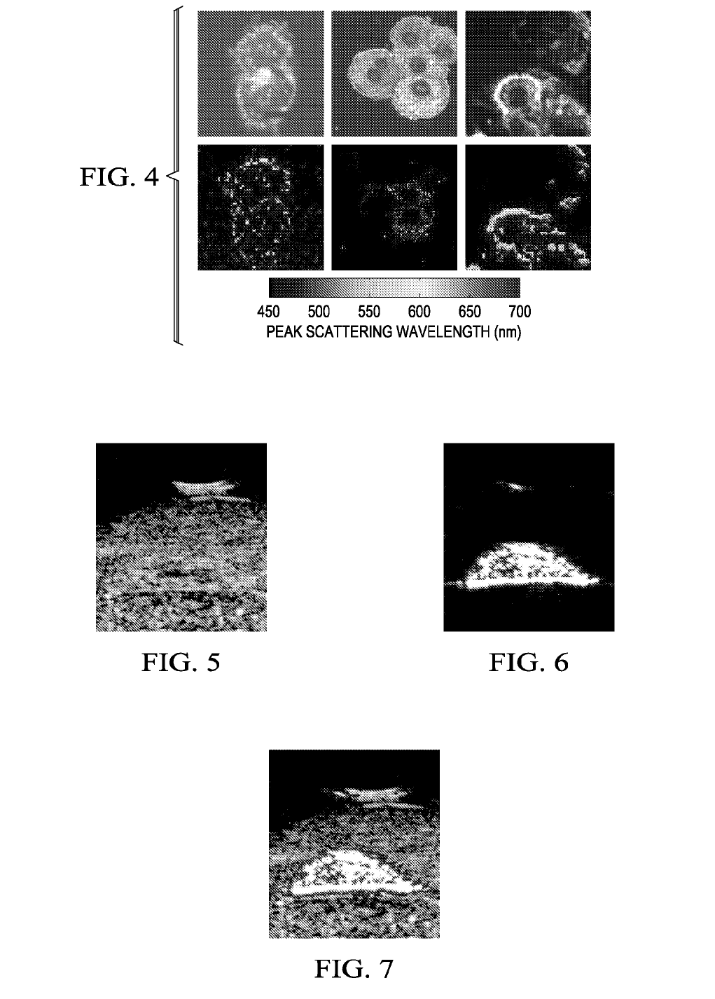

FIGS. 5 and 6 and 7 are photoacoustic imaging of coated biodegradable gold nanoclusters. The particles used in the photoacoustic images from Soon Joon were lysine nanoclusters with PLA(2 k)-PEG(10 k)-PLA(2 k) biodegradable polymer. They were 100% evaporation clusters with a size of 80-90 nm (same size as the 100% clusters we reported in the bionano papers).

The present invention provides photoacoustic imaging of nanoclusters in tissue phantoms. The phantoms were prepared using a mixture of 8% gelatin by weight and 0.1% 10 μm silica particles. The silica particles provided ultrasonic scattering. First, a thick layer of the gelatin/silica particles mixture was formed on bottom of a well. Then, a drop of nanoclusters mixed with the same gelatin/particle suspension was placed on top of the first layer and was allowed to gel. Finally, another layer of gelatin/silica particle mixture was added on top. Photoacoustic and ultrasound imaging was carried out using a single element focused ultrasound transducer and a pulsed laser system. The laser light was delivered using the integrated probe consisting of several optical fibers positioned around the ultrasound transducer. Nanoclusters were not visible in ultrasound image FIG. 5 but provided high contrast in photoacoustic image FIG. 6. Since the images were collected using the same ultrasound transducer, these images are spatially coregistered and could be overlaid one on top of each other FIG. 7. Clearly, these results demonstrate that NIR absorbing nanoclusters may act as contrast agents for photoacoustic imaging.

The small gold nanoparticles of the present invention can be assembled together into nanoclusters ˜100 nm in diameter using biodegradable polymers. Tight packing of primary particles in the nanoclusters results in strong NIR extinction. The nanoclusters are stable at physiological pH and deaggregate in pH environment that mimics lysosomes down to essentially primary nanoparticles with 4 nm gold core diameter. Furthermore, the nanoclusters deaggregate in live cells over time.

The present invention describes a method to synthesize gold nanoclusters with controlled size, shape, gold packing fraction and strong NIR absorbance that will biodegrade into individual gold nanoparticles smaller than about 5.5 nm in in vitro assays and in animal models in vivo. The nucleation and growth of the clusters was controlled by varying the gold concentration, ligands on the gold surface, polymer/gold ratio, polymer architecture, pH, solvent evaporation rate and extent, and use of secondary polymer stabilizers. These rates and the interactions between the capping ligands influence the density and size of the nanoclusters. The present invention further describes methods for conjugation of antibodies and targeting peptides onto either gold or the stabilizing polymers on the nanoclusters, for molecular specific targeting of cancer cells.

The ligands on the gold nanocrystals and the polymers was designed to provide sufficient interparticle attraction to favor the formation of tight clusters, to give the desired NIR optical properties. For low molecular weight capping ligands on gold that produce strong interparticle cross-linking interactions such as cysteine, lysine, and glutathione, polymer stabilizers including PLA(1K)-PEG(10K)-PLA(1K), PLA(1K)-PEG(5K)-PLA(1K), and PLGA(1K)-PEG(10K)-PLGA(1K) were used to weaken these interactions, so that the clusters do not grow too large. For ligands that do not produce strong interparticle interactions including citrate and glutamic acid, the polymers were used to aid gold clustering.

In order to vary particle charge, various stabilizing ligands, such as citrate (negative charge), lysine (zwitterionic), glutathione, cysteine (zwitterionic), PEG-SH (neutral), and 2-aminoethanethiol (positive charge) were investigated in the synthesis of primary gold nanoparticles. Aqueous solutions of these ligands were prepared, and then added to an aqueous solution of HAuCl₄ at approximately 97° C. The gold was reduced with NaBH₄. Particle surface charge was assessed by zeta potential measurements, which indicated an approximately neutral charge (zeta potential of 2 mV) on the cysteine particles and citrate: zeta potential of −44 mV, lysine zeta potential of −30 mV and glutathione zeta potential of −46 mV. These particles were then used to form clusters with the biodegradable polymer PLA(2K)-PEG(10K)-PLA(2K).

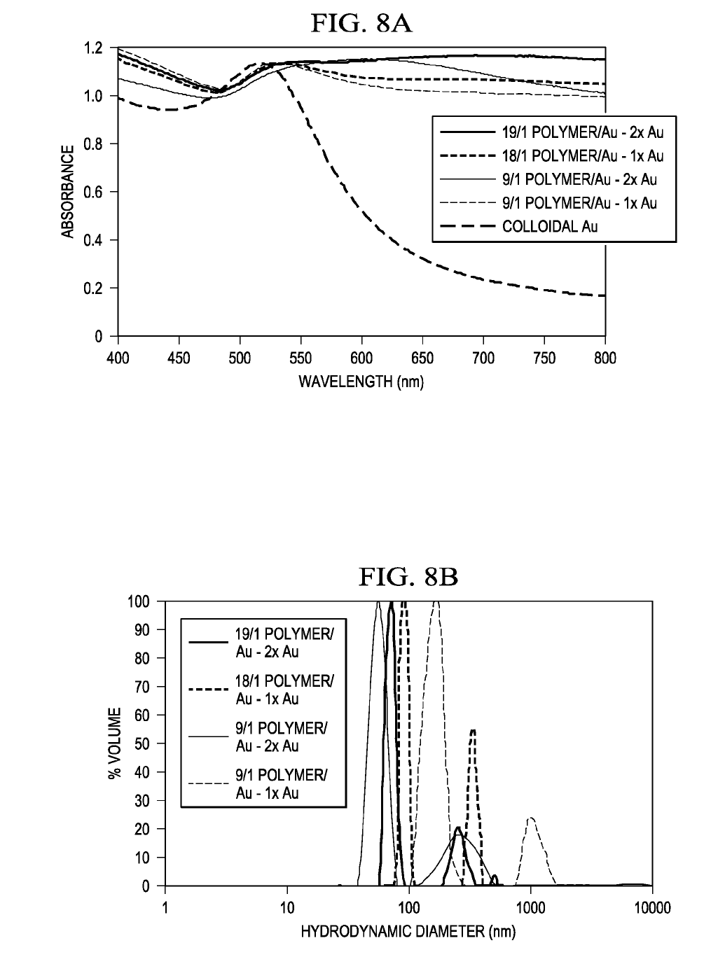

A variety of variables were manipulated in the present invention to control the nucleation rate and growth rates that determine the cluster morphology. For example, rapid nucleation and slow growth will favor small clusters, whereas the solvent evaporation will produce the desired small interparticle spacings for the gold. The initial concentration of gold nanoparticles was varied from 0.5 to 5 mg/mL, and the polymer/gold ratio from 4/1 to 40/1. For lysine, strong NIR absorbance was achieved for gold concentrations of 3 (2× loading) and 1.5 mg/mL (1× loading) for polymer gold/ratios of 9/1 to 19/1 (FIG. 8). Whereas nanoclusters on the order of 100 nm, by DLS, were formed for each of these conditions, the smallest size was found at 2× gold and the lower polymer loading. The higher gold concentration appeared to raise the nucleation rate. The kinetics was also manipulated by varying pH, and salt concentration.

The present invention also includes method to induce cluster formation by evaporation of the solvent. The loss of hydration of the polymer stabilizers as the last 20% of the water is removed may be expected to cause polydispersity in the cluster size. The solvent was evaporated partially (50 to 90%) to induce nanocluster formation and the solution was be flash frozen and lyophilized, rapidly filtered within a few minutes by tangential flow filtration to remove the remaining solvent or the clusters were quenched by the addition of hydrophilic stabilizers including polyethylene glycol and polyvinyl alcohol to stop the particle growth. The temperature was varied from 50 to 3° C. to control the cluster growth. Another approach includes adding ethanol to the water to influence the nucleation rate. Yet another approach involves allowing the gold particles to undergo partial clustering and then quench with polymer(s).

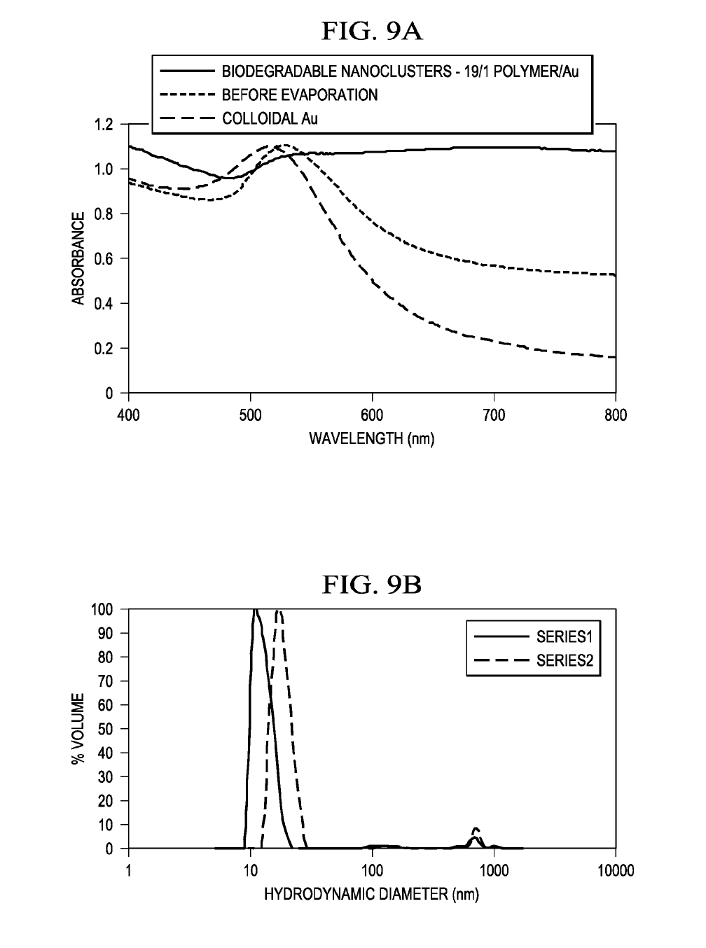

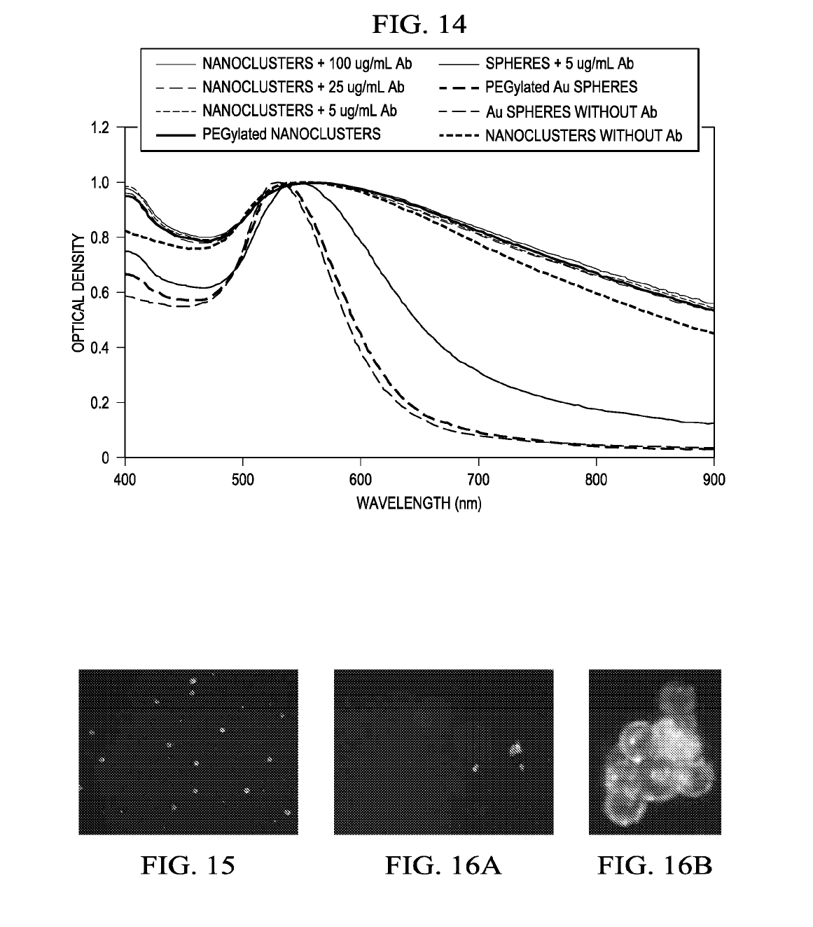

The clustering of the gold particles was further manipulated by the rate and amount of water evaporation. The modest red-shift in the extinction spectrum of gold nanoclusters for the lysine-PLA(2K)-PEG(10K)-PLA(2K) system indicated limited clustering of the gold primary particles without water evaporation (FIG. 9), as confirmed with very small nanocluster sizes shown by DLS (FIG. 14A). After evaporation to raise the particle concentration and manipulate the polymer solvation and ion hydration, the presence of the NIR peak indicates the cluster formation (FIGS. 8 and 9) as was also seen by DLS.

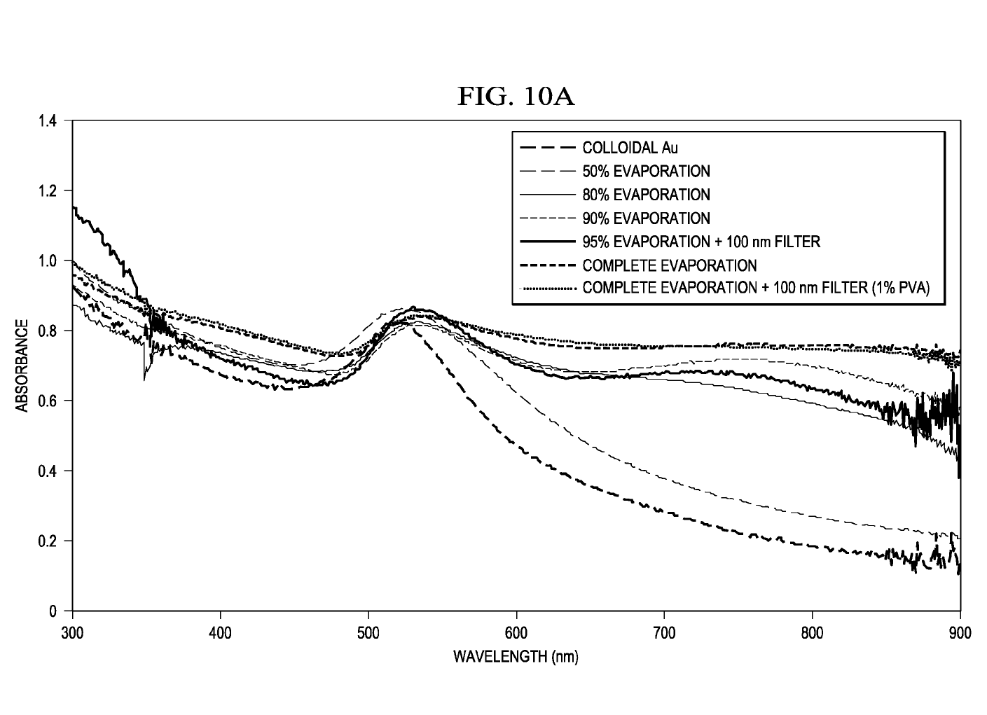

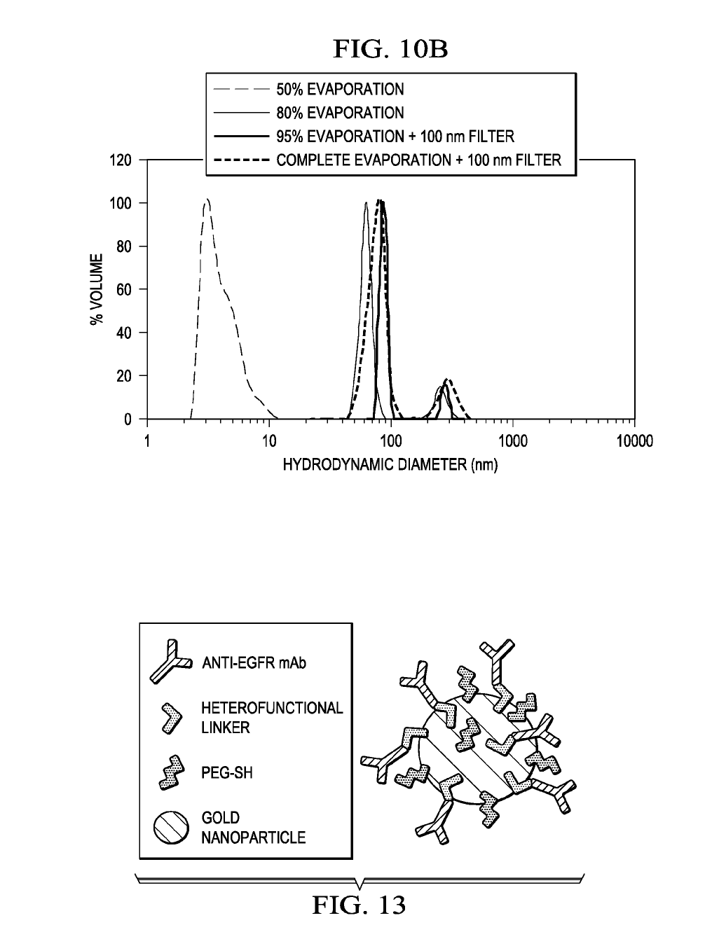

FIGS. 10A and 10B show that when only 50% of the liquid is evaporated, nanoclusters are not formed, as evidenced by only a modest shift of the UV-vis curve and cluster sizes less than 10 nm. However, as the extent of evaporation reached 80%, a second peak in the NIR region (>700 nm) of the UV-vis spectrum began to appear (FIG. 10A) and an increase in particle size was observed, between 40-80 nm (FIG. 10B). As the extent of evaporation further increased towards 100% evaporation, or complete evaporation to form a dry film, the second peak in the UV-vis curve became more prominent. The cluster size also increased marginally, between 50-110 nm, with an increase in the extent of evaporation.

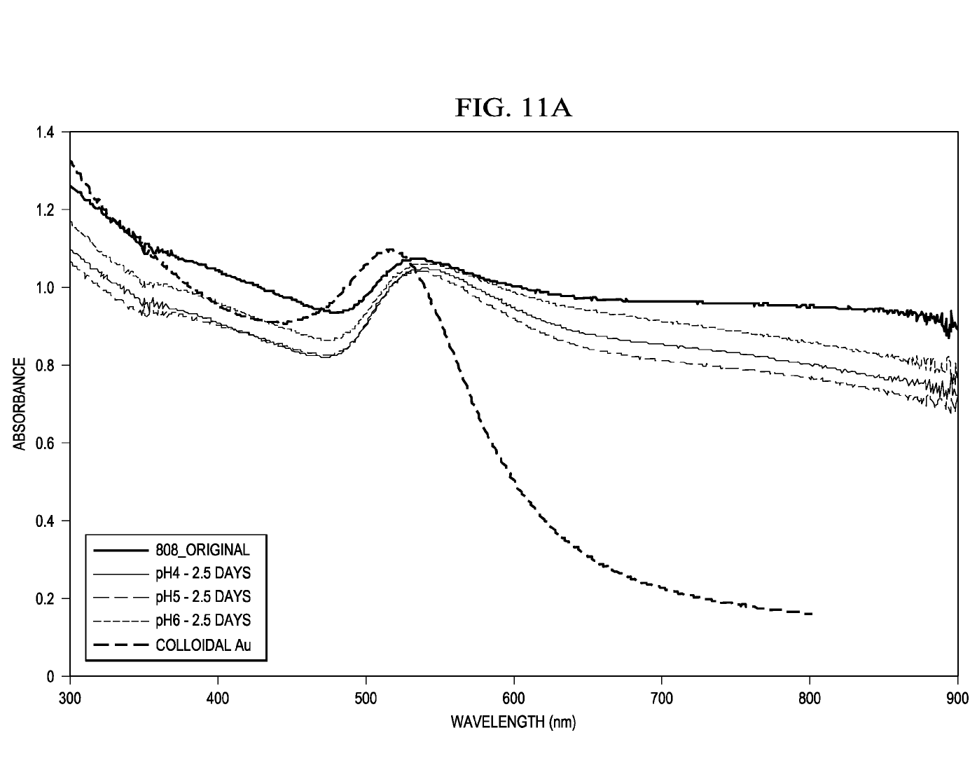

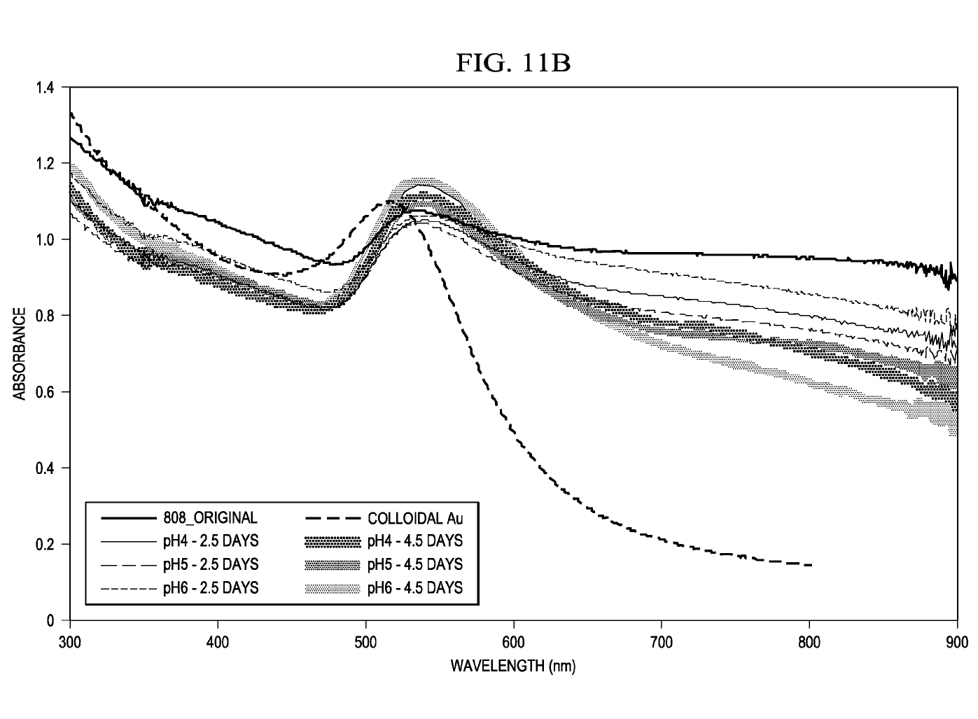

The pH range over which nanocluster deaggregation occurs was also examined. FIGS. 11A and 11B show that significant nanocluster deaggregation occurs in pH 6 media, as seen by the decrease in the NIR peak in the UV-vis absorbance curves. FIG. 11A clearly shows that nanocluster deaggregation occurs more rapidly in lower pH media, either pH=4 or 5. After 4.5 days of incubation, significant deaggregation is observed in pH=4, 5, and 6 media. Therefore, these nanoclusters are capable of deaggregating inside cells, which include organelles with pH varying between 5 and 6.

As previously described the nanoclusters in the present invention were characterized by UV-vis-NIR spectroscopy, SEM, TEM, STEM-EDX, and DLS. In addition to the above mentioned methods small angle X-ray scattering (SAXS) to determine the gold nanoparticle distribution functions, zeta potential measurements in a DLS apparatus to determine cluster charge, and thermal gravimetric analysis (TGA) to determine the polymer/gold ratio and BET adsorption measurements of the particle porosity were also done. For the SEM analysis the clusters were fixed with an epoxy prior to evaporation of the water, and then microtoming the sample. This approach enabled more accurate identification of clusters in solution prior to drying, and facilitated comparisons of cluster size by SEM and DLS. Furthermore, the absorbance and hydrodynamic diameter as a function of time at conditions (low T, certain pH ranges) where the particles are stable to find the optimum environment for particle storage was measured. The absorbance was also determined during the solvent evaporation protocols to understand the clustering kinetics.

To complement the measurements of hydrodynamic diameter by DLS, the zeta potential was measured with a Zetaplus to understand the influence of charge on the clusters on their colloidal stability as a function of pH, ionic strength, and the polymer/gold ratio. For example, in the case of lysine at a pH above 5, the two positive charges in addition to the single negative charge provides a net positive charge to provide electrostatic stabilization to complement the steric stabilization from the polymer.

The mass of gold particles per volume of solution was determined from flame atomic absorption spectroscopy (AA). The total volume of gold per volume of solution was determined from this mass and the known density of gold. The number of gold particles per volume of solution was determined from the mass of gold per volume, the gold diameter (TEM) and the gold density. The mass of polymer/mass of gold was be determined by TGA, and used to determine the mass and volume of polymer per volume of solution. The porosity of the nanoclusters was determined with a BET adsorption apparatus. The volume average nanocluster size and size distribution was determined by DLS. From these properties the effective number of nanoclusters per volume and number of gold particles per nanocluster, and the volume fractions of gold and polymer in each nanocluster was determined.

SAXS measurements were used to determine the average center-to-center separation of the gold particles within the clusters, as has been reported previously for gold clusters. The measurements are made with X-rays from a rotating copper anode generator. The generator is operated at 3.0 kW and the scattered photons are collected on a two-dimensional multiwire gas-filled detector.

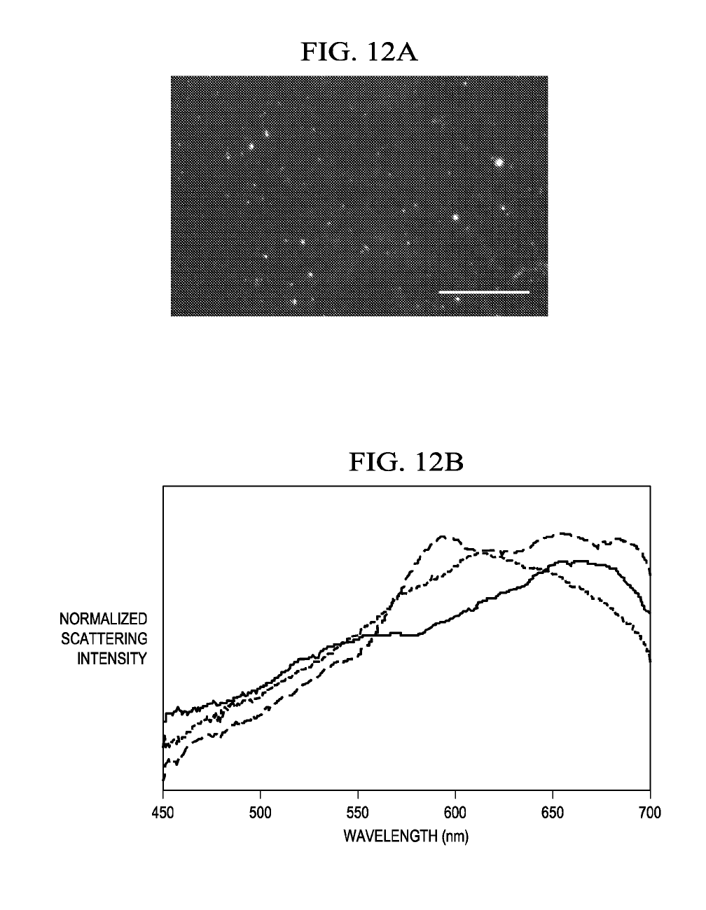

In addition to UV-Vis/NIR spectroscopy, the scattering and absorbance of individual nanoclusters was measured using hyperspectral microscopy (FIG. 12). A prism based PARISS hyperspectral imaging device (LightForm, Inc.) coupled to a Leica DM6000M microscope with broad band excitation provided by either halogen or Xe lamps was used. The system was designed to detect signals from 350-850 nm with 1 nm spectral resolution. These studies determined the homogeneity of optical properties of the nanoclusters.

The nanoclusters of the present invention were conjugated with monoclonal antibodies for the epidermal growth factor receptor (EGFR)—an important cancer biomarker which is associated with carcinogenesis in many epithelial cancers including lung, oral cavity, and cervix. A large fraction of the gold surface was available for conjugation given the relatively weak binding of PLA and PLGA to gold and the low molecular weights of the polymer stabilizers.

Antibodies were attached to the gold surfaces in the nanoclusters via a conjugation linker that consists of a short polyethylene glycol (PEG) chain terminated at one end by a hydrazide moiety, and at the other end by two thiol groups. Antibodies at a concentration of 1 mg/mL were be exposed to 10 mM NaIO₄ in a 40 mM HEPES pH 7.4 solution for 30-40 minutes at room temperature, thereby oxidizing the hydroxyl moieties on the antibodies' Fc region to aldehyde groups. The formation of the aldehyde groups were colorimetrically confirmed using a standard assay with an alkaline Purpald solution. Excess hydrazide-PEG-thiol linker was added to the oxidized antibodies and allowed to react for 20 minutes. The hydrazide portion of the PEG linker interacts with aldehyde groups on the antibodies to form a stable linkage. In this procedure a potential loss of antibody function is avoided because the linker cannot interact with the antibody's target-binding region, which contains no glycosylation. The unreacted linker was removed by a 100,000 MWCO centrifugal filter (Millipore). After purification, the modified antibodies were mixed with gold nanoparticles in 40 mM HEPES (pH 7.4) for 20 minutes at room temperature. During this step a stable bond is formed between the gold surface and the linker's thiol groups. Subsequently, monofunctional PEG-thiol was added to passivate the entire nanoparticle surface (FIG. 13). The hydrophilic nature of PEG improves the biocompatibility of the conjugate. The conjugates are centrifuged and resuspended in 1×PBS. The amount of antibodies per particle can be controlled by simultaneous exposure of gold surface to the mixture of the proteins and PEG-thiol molecules at different stoichiometric ratios.

Antibody targeted nanoparticles can have decreased blood circulation times as compared to particles conjugated with molecular specific peptides. However, peptides have some drawbacks including lower binding constant and decreased conformational stability in comparison with antibodies. Furthermore, directional attachment of antibodies through Fc portion shown in FIG. 13 would decrease recognition by macrophages in liver and spleen because these cells recognize antibodies through Fc moiety. In addition, the presence of PEGylated templating polymers should provide a strong “stealth” properties to the nanoclusters thus extending their circulation time that is need for accumulation in tumor.

Gold nanoclusters were conjugated with anti-EGFR antibodies in order to target them to cells. Anti-EGFR antibodies were first suspended in 40 mM HEPES (pH 8) and then mixed with NaIO4. This oxidation reaction was quenched with phosphate-buffered saline (PBS) solution and added to 50 mM PEG-dithiol linker in order to add the linker to the antibody. Conjugation of the nanoclusters was performed by adding anti-EGFR-PEG-dithiol prepared previously to a solution of nanoclusters (approx. 10¹⁰ particles/mL) and mixed for approximately 24 hours in order to facilitate antibody binding to the gold surface of the nanoclusters. mPEG-SH in water and then 2% by volume of PEG in phosphate-buffered saline were then added, and the resulting product was centrifuged and resuspended in DMEM cell media in order to facilitate a cell targeting test. A431 (lung cancer) cells were taken from the incubator and mixed with anti-EGFR conjugated nanoclusters in order to facilitate cell labeling. Cells were then viewed under a darkfield reflectance (DF) microscope.

Absorbance spectra taken before and after conjugation of the antibody to the nanocluster and the control gold nanospheres and nanoclusters without conjugated antibody show minimal aggregation and continued NIR absorbance upon addition of antibody, as can be seen in FIG. 14. FIG. 14 is a graph of the absorbance spectra of nanoclusters and solid Au sphere before and after addition of various concentrations of anti-EGFR antibody.

Cell targeting was assessed by comparing darkfield reflectance images of cells incubated with PEGylated control nanoclusters with images of A431 cells incubated with anti-EGFR conjugated nanoclusters. FIG. 15 is an image of A431 cells incubated with PEGylated nanoclusters without antibody conjugation. FIG. 16A is an image of A431 cells incubated with nanoclusters conjugated with 25 ug/mL Ab. FIG. 16B is an close-up of image in FIG. 16A showing gold within A431 cells. This comparison shows a clear contrast between the control cells, which did not have gold inside them, and the cells with conjugated nanoclusters, which had gold inside them, showing a significant amount of cell targeting.

The present invention also describes engineering optimization of composite nanoparticles through the evaluation the following composite particle features: overall particle shape, whether a filled-sphere, or a hollow spherical shell (with enhanced payload capacity); composite particle packing method and associated bulk-production methodology, variations between maximally-jammed-packing (MJP) and diffusion-limited-growth (DLG) type packing; and mean interparticle-distance and mean total particle number. Design of optimal structures may also be facilitated through the use of theoretical and computational-physics based modeling of desired electrodynamic properties.

The overall red-shift of the extinction cross section can be expressed in terms of statistical features of an aggregate, such as its overall dimensionality (i.e. whether 2D or 3D in shape), its total number of particles, and its mean inter-particle spacing. For aggregates comprised of a very large number of particles, which is the case in the present invention, an additional effect which must be considered involves the transition to an effective-medium. The collective-mode effects associated with this effective-medium induce additional modifications to the formulas applicable to smaller aggregates. Most importantly, these latter effective-medium considerations indicate that the manner in which the composite particles are packed into larger structures, for example, whether a maximally-jammed-packing, or a diffusion-limited-growth type of packing are used, critically affects the optical properties of the composite structure.

The present invention also describes studies to test stability and deaggregation of both non-targeted and molecular specific anti-EGFR nanoclusters in a variety of solutions that mimic environment of cellular organelles (lysosomes) and tissue. The invention further details the interaction of nanoclusters with living cells and cell mediated deaggregation process

The extracellular pH in tumors is more acidic than in normal tissue, whereas the intracellular environment is neutral or slightly alkaline. This pH gradient is opposite that for normal cells. For example, for human malignant head and neck tumors, the intracellular pH ranges from 7.07 to 7.25 whereas the extracellular values range from 6.58 to 6.9, on the basis of measurements with fluorescent dyes. The elevated cytosolic pH is maintained by enhanced sequestration of cytosolic protons into the acidic cellular vesicles including endosomes and lysosomes. The pH of 4.6 to 5.0 in the interior of lysosomes, with sizes ranging from 0.1 to 1 μm, and the degradative hydrolytic enzymes, will aid biodegradation of the polymers in the nanoclusters, for example, for polyesters including PLA and PLGA. For human breast cancer cells, the extracellular acidification been shown to move lysosomes toward the cell periphery and to increase the number of lysosymes

The composition, size, surface charge, and type of targeting molecules on the nanoclusters was varied to influence the cellular uptake and degradation. The particle size was examined from 50 to 150 nm as it plays a key role. For example, PLGA nanoparticles smaller than 100 nm exhibited 27-fold higher gene transfection than those larger than 100 nm. The surface charge of the nanoclusters will be adjusted by varying the concentrations of the ligands on the gold surface (negative for citrate and positive for lysine) and the polymer (negative for PLGA at neutral pH from the end carboxylic acid groups, but neutral in acidic lysosomes).

FIG. 16 shows that gold nanoparticles capped with negatively charge citrate ligands form nanoclusters in the presence of PLA-PEG-PLA during solvent evaporation (t=0). Furthermore, these clusters degrade nearly completely to gold particles in 4 days at pH 4. The acidic conditions were needed only to degrade the polymer and had little effect on the negatively charged citrate coated particles that disperse as primary particles.

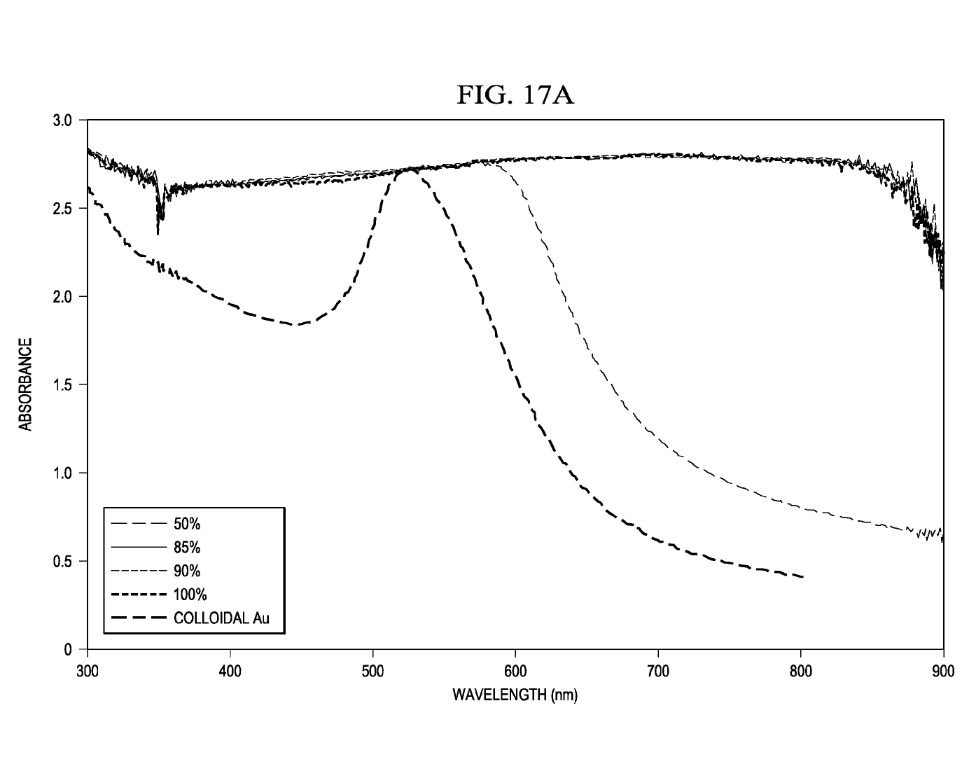

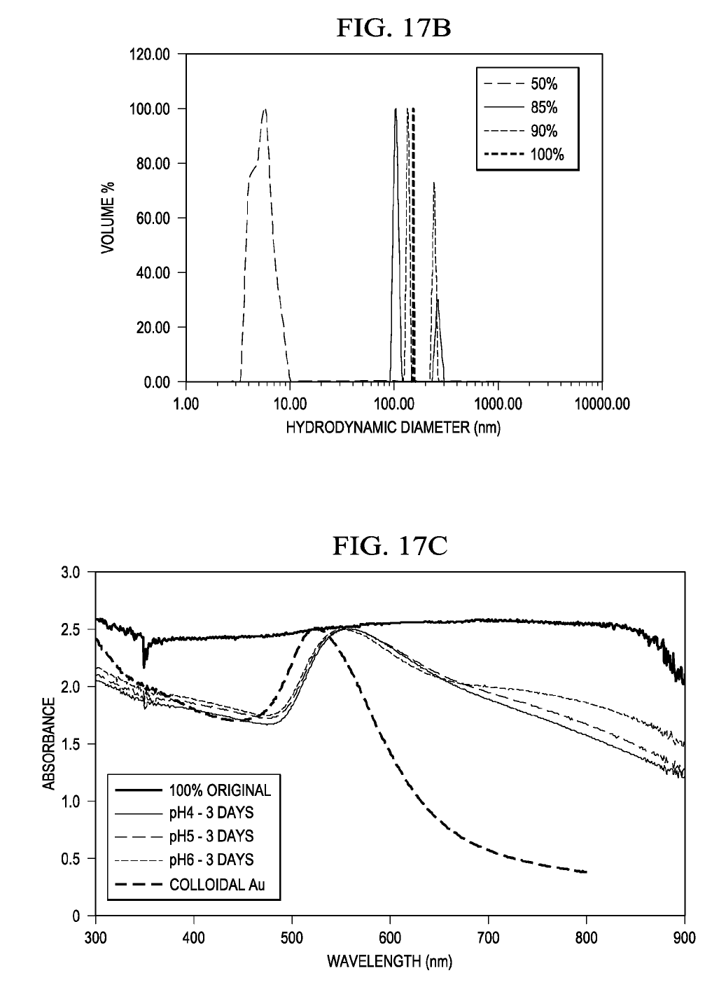

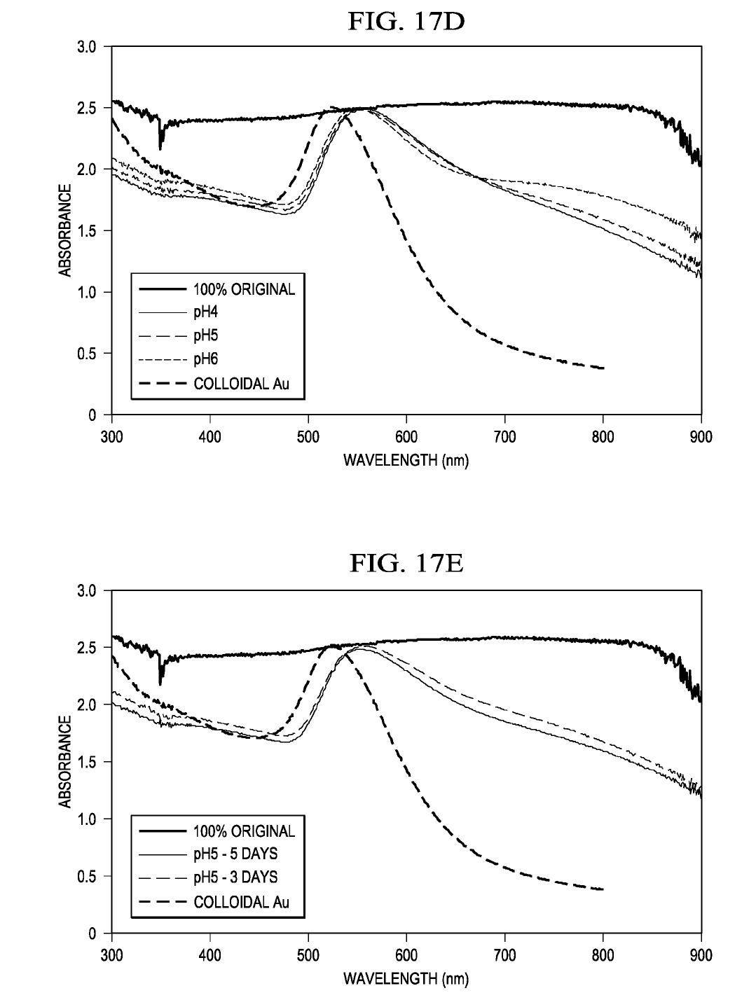

Natural biodegradable polymers such as alginic acid were used to control nanocluster formation and deaggregation. Gold nanoclusters can be formed using a double emulsion templating process in which lysine stabilized gold nanoparticles makes up the inner water phase, dissolved polymer makes up the organic phase, and an aqueous alginic acid solution makes up the outer water phase. As an example, a nanocluster using PLA(2K)-PEG(10K)-PLA(2K) polymer and the polymer/gold ratio of 16/1 was synthesized. The alginic acid solution concentration was varied between 1 and 0.1% w/v to yield polymer/gold/alginic acid ratios of 16/1/20 and 16/1/2, respectively. Only the composition with 0.1% w/v alginic acid solution was shown to deaggregate significantly after 1 week (FIG. 17).

The use of pH-sensitive, low molecular weight ligands to modulate gold nanocluster aggregation and deaggregation was demonstrated using glutathione (a tripeptide consisting of glycine, cysteine, and glutamic acid), cysteine, glutamic acid, in addition to lysine. In previous studies, the nanocluster aggregation was not controlled and thus the clusters grew to sizes much larger than 100 nm. The present invention controls the cluster size with the addition of biodegradable polymers to inhibit cluster growth as shown for nanoclusters with lysine capping ligands. The above series of capping ligands possess different surface charges at neutral pH: lysine is positively charged, while glutathione is negatively charged, and cysteine is zwitterionic. The variation of surface charge on the primary gold particles is of interest as it has been found to influence the rate of renal clearance.

Nanocluster deaggregation rates will also be influenced by the polymer degradation rate. Our preliminary experiments used PLA(2K)-PEG(10K)-PLA(2K). PLA (MW=2K) has a half life of 4 weeks at pH 7, but decayed much more rapidly at pH 4. PLGA (poly(lactic-co-glycolic acid)) blocks of similar length will increase biodegradation rates, as glycolide units degrade more rapidly than lactide units. The tri-block PLA-PEG-PLA will be compared with di-block PLA-PEG, as the different polymer structure may influence particle packing and thus affect deaggregation rates. Stability and non-specific interactions with proteins that can significantly alter both deaggregation process and the size of nanoclusters.

The present invention describes the development of a new technological platform for creation of plasmonic and hybrid multimodal/multifunctional nanoclusters which will undergo biodegradation and accelerated clearance in vivo. This present invention removes one of the most significant roadblock in translation of plasmonic and other types of nanoparticles to the clinic—concerns of long term toxicity. The development of biodegradable plasmonic nanoclusters described in the present invention will provide an opportunity for an accelerated translation of this technology to phase I and II clinical trials in human subjects.

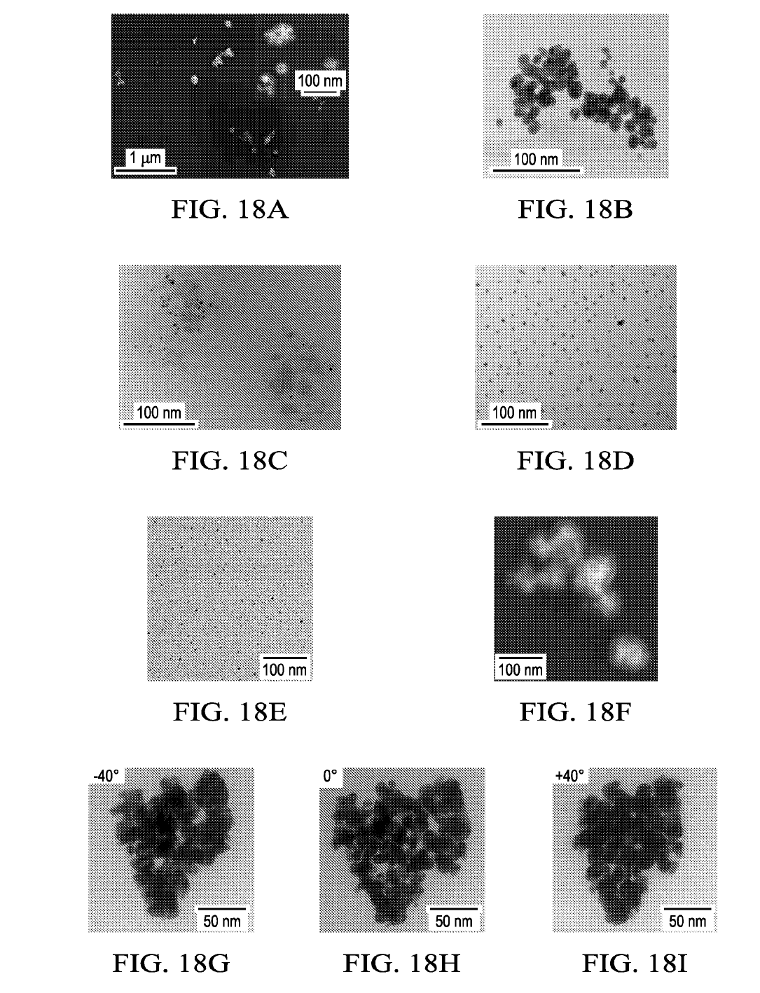

FIGS. 18A-18I are images of Cluster growth is controlled through mediation of the interactions between ligand-capped gold particles with the biodegradable polymer. Gold nanoparticles stabilized with citrate ligands were synthesized. A solution of 1% lysine in pH 8.4 phosphate buffer (10 mM) solution was added to 1.2 mL of a 3.0 mg/mL colloidal gold solution to yield a final lysine concentration of 0.4 mg/mL and an average diameter of 4.1±0.8 nm. The dispersion was stirred for 12 hours. PLA(2K)-PEG(10K)-PLA(2K) (60 mg) was added to the aqueous gold dispersion, yielding a final polymer concentration of 50 mg/mL. The dispersion was sonicated in a bath sonicator for 5 minutes, during which the dispersion changed from ruby red color to a darker red-purple color. Upon evaporation of ˜80% of the solvent, the dispersion turned blue, indicating absorption in the red. Complete solvent evaporation over two hours produced a smooth blue film. Reconstitution of the film with deionized (DI) water to a concentration of ˜0.3 mg/mL, yielded a dark blue dispersion. The fact that this dispersion consists of sub-100 nm clusters composed of primary gold nanoparticles is indicated by scanning electron (SEM) and transmission election microscopy (TEM). TEM images taken at various angles reveal closely-spaced primary gold nanoparticles throughout the porous cluster. The average hydrodynamic diameter measured by dynamic light scattering (DLS) was 83.0±4.6 nm, in agreement with the TEM results. In the SEM image a polymer-rich shell a few nanometers thick is apparent on the exterior of the clusters, which potentially provides steric stabilization of the dispersion.

The nanoroses and nanoclusters of the present invention can be used alone or in combination with an active agent to deliver an active agent payload to a target cell. Often, the active agent may be released based on the degradation of, e.g., a controlled release biodegradable matrix and/or polymer. However, it has been found that the nanoclusters of the present invention can also deliver their payload by laser heating, magnetic or optical disruption of the nanoclusters.

The nanoroses and nanoclusters can be coated with dextran to target the macrophage cells, since macrophages have dextran receptors. Uptake of nanoclusters into macrophage cells associated with tumors, atherosclerosis, and arthritis is investigated with dark field and phase contrast microscopy. The nanoclusters optical properties within macrophages were measured with hyperspectral microscopy. In addition, a localized temperature increase, obtained during the irradiation of 755 nm single pulse infrared laser therapy, was monitored using a point infrared detector. The thermal ablation was evaluated through the absorption effectiveness of nanoclusters after uptake by macrophages in vitro.

The nanoclusters of the present invention can be adapted for administration using a wide variety of methods of delivery, including, but not limited to, e.g., subcutaneous, intraveous, peritoneally, orally, intramuscular, topical, nasally, intradermal, ocular, rectal, vaginal and combinations thereof. The nanoroses can be used in patients who have previously received a drug eluting stent, as a method to identify polymers on stents causing a localized inflammatory reaction. The predominant cell type in these inflammatory reactions are macrophages, and if identified, place that drug eluting stent at greater risk for acute stent thrombosis (heart attack for the patient). Thus, patients who have drug eluting stents who have concerns regarding late stent thrombosis could have tunable optical nanoparticles injected intravenously prior to heart catheterization, to determine if there are macrophages infiltrating around the stent struts. This approach can be coupled with the use of intensity sensitive OCT to detect the anatomic marker of late stent thrombosis, which is retraction of the vessel wall from the stent struts. If these findings are present, then anticoagulation with certain agents such as but not limited to Plavix, would be prolonged to mitigate against acute stent thrombosis in the future

The nanoroses of the present invention can also be used not only for detecting, but also for treating macrophage laden plaques with the same nanoparticle. Macrophages in atherosclerotic plaques are known to be an important risk factor for heart attacks. Thus, spectrally-tunable optical nanoparticles permit not only the identification of macrophages as a marker of vulnerable plaque, but may also be used to treat these macrophages as well at the time of identification. By extending the intensity of laser exposure, additional heating of the nanoparticles can be accomplished to transition the macrophages into apoptosis. The nanoroses can use used as part of a treatment regimen for the selective elimination of plaque based macrophages via apoptosis as a method to stabilize vulnerable plaque. The transition to apoptosis can be accomplished with less than a 5° C. elevation of temperature, far from the 50-60° C. elevations in temperature seen with traditional laser angioplasty as practiced for the last two decades.

A further application of the nanorose can be to prevent cancer from metastasizing to other locations in the body. Aggressive cancers are known to induce an inflammatory response composed of macrophages. These macrophages which initially attack the tumor (M1 phenotype) evolve to a tumor supportive role within the tumor environment (M2 phenotype). M2 macrophages encourage angiogenesis and break down basement membranes, both critical factors in allowing tumors to metastasize. IV injection of nanorose provides a means to have nanorose uptake in tumor associated macrophages (TAM). The use of laser energy would allow selective necrosis and vaporization of these TAM, transitioning aggressive tumor phenotypes to more benign tumors which could then be cured with local resection.

Gold nanoparticles that absorb in the near infrared (NIR) offer abundant opportunities for minimally invasive optical imaging and photothermal treatment of cancer and atherosclerosis. The present invention includes ˜30 nm clusters of iron oxide@gold core shell primary particles with intense NIR absorbance from 700 to 850 nm in aqueous media and primary mouse peritoneal macrophage cells. Kinetic control of the aggregation produces relatively uniformly-sized particles with stable NIR absorption in aqueous media for 6 months, despite the unusually small size and high surface area. The small size of the clusters and the dextran coating facilitate rapid and strong uptake by the macrophage cells, with up to 3000 nanoroses per cell. As a consequence of the large optical density of 0.6 within each cell, as shown by hyperspectral microscopy at 755 nm, a single 50 ns laser pulse is sufficient to produce photothermal ablation.

Gold plasmonic nanostructures are receiving great attention as contrast agents for in vivo optical imaging of tissue with optical coherence tomography, photoacoustic tomography and two-photon luminescence in atherosclerosis and cancer. The depth of penetration of tissue may be improved by tuning the gold surface plasmon resonance (SPR) into the NIR (700-850 nm), where soft tissue, hemoglobin and water are the most transparent. The SPR of gold undergoes a red shift into the NIR region in confined geometries including nanoshells, nanorods, nanocages and clusters of gold primary particles. Gold nanospheres bioconjugated with antibodies have been assembled by cancer receptors within cells to form clusters with high NIR contrast ratios for precancerous versus normal cells.

The selective delivery of gold nanoparticles to targeted cells and eventual clearance from the body have been shown to improve with a decrease in particle size. Ultrasmall 20 nm nanoparticles may be used to target lymph-node-resident dendritic cells for vaccine delivery. Recently, 40-50 nm particles were found to be optimal for nanoparticle-mediated binding of membrane receptors for signaling a variety of cell functions including cell death. To design these ultra-small nanostructures, several challenging must be addressed. As the size reaches 30 nm and smaller, the red shift to the NIR often vanishes. Furthermore, because of the high surface energy, the particles often do not form stable dispersions in various physiological media, or may undergo changes in shape to reduce the surface area. Finally, the gold domains and polymeric surface coatings, utilized for particle stabilization and cell targeting, must be packed into a very small overall particle volume.

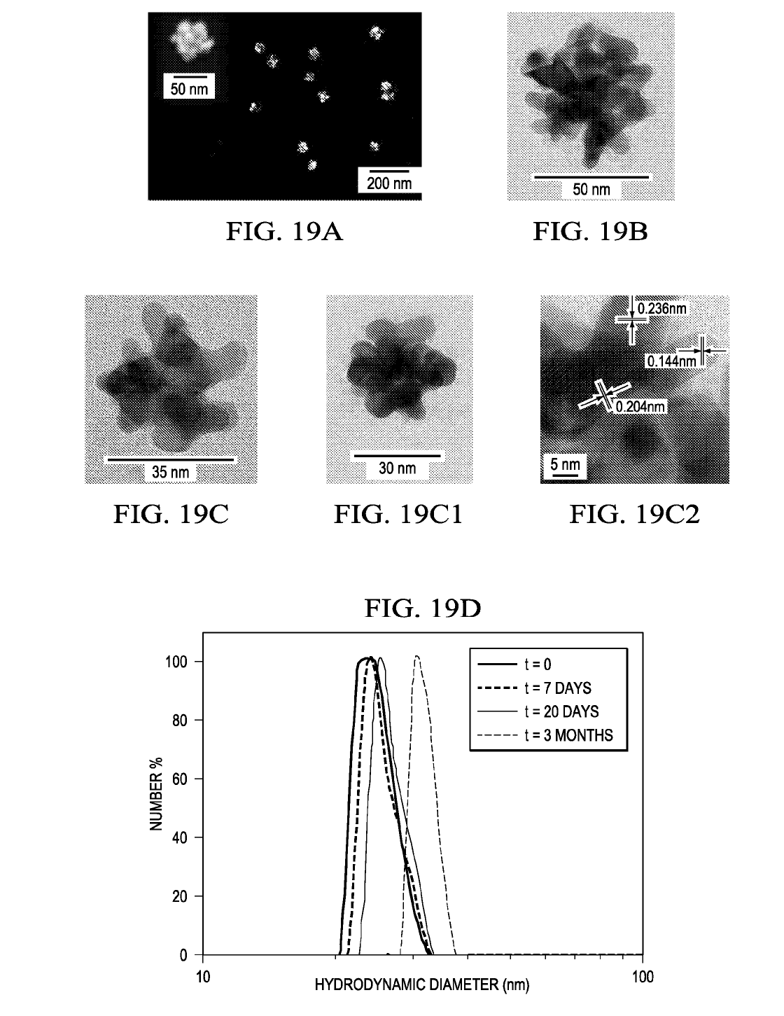

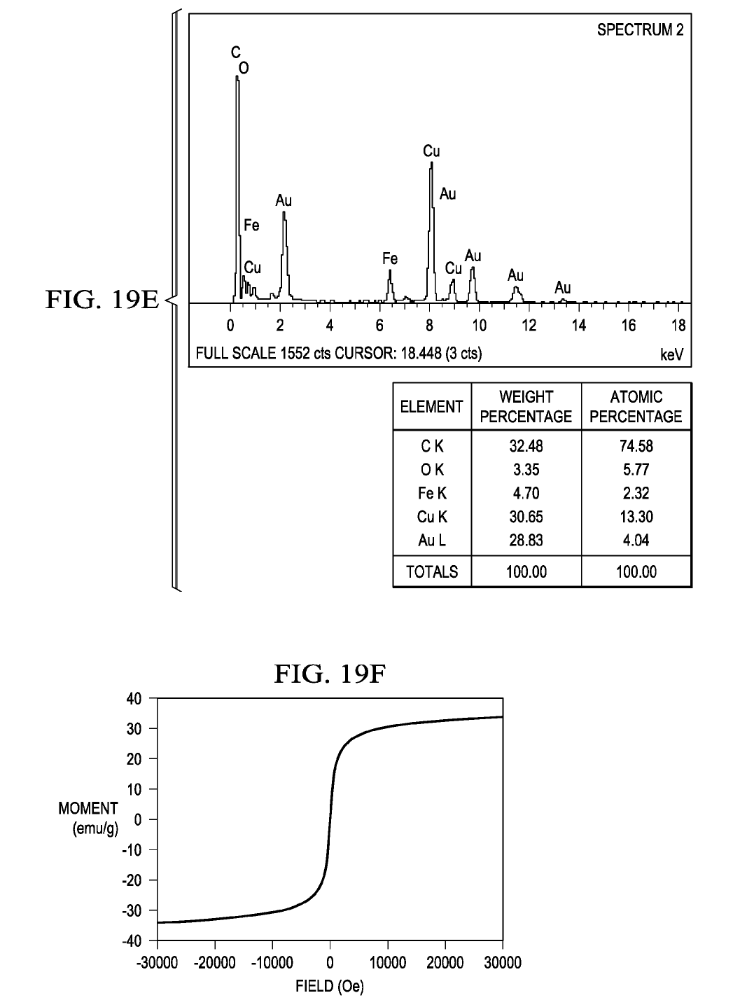

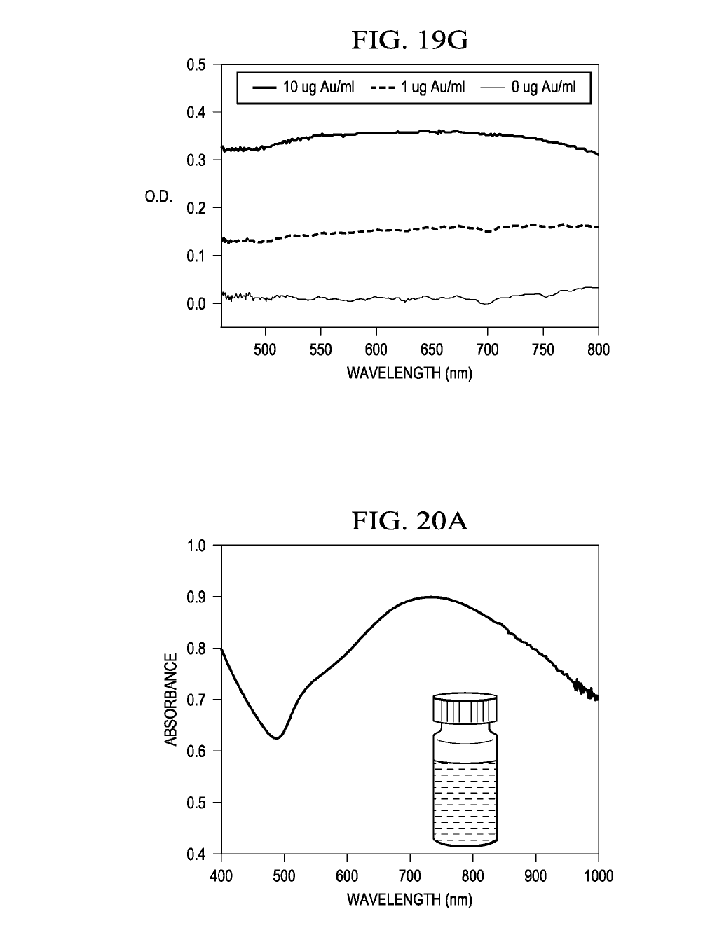

Surprisingly, the present inventors were able to make nanoclusters that are unusually small and stable ˜30 nm (based on dynamic light scattering) cluster of iron oxide gold shell primary particles with an open structure as shown in FIG. 19 with strong SPR in the NIR region. For simplicity, the nanocluster is also referred to as a “nanorose” based on FIG. 19. The use of the term “rose” is neither a limitation of the present invention, nor a requisite shape of the nanoclusters of the present invention, it is merely used as a short cut to distinguish the shape from other nanoparticles, such as, nanoshells, nanorods and nanocages. A strong absorption cross-section in the NIR between 700 and 850 nm is produced by a combination of the close proximity of the gold nanoparticles, the open and non-spherical shape of the clusters, and regions of thin gold shells on the iron oxide cores. We controlled the aggregation kinetically, as a function of gold shell growth rates and amount of dextran stabilizer, to obtain the small ˜30 nm clusters with relatively low polydispersity and favorable geometry. In contrast, the reported NIR absorbance has been weak for individual (non-clustered) iron oxide gold shell particles with diameters of 6 to 60 nm.

The small particle size and presence of dextran on the nanorose surface is shown to facilitate high uptake into macrophage cells, resulting in strong contrast enhancement in cellular imaging and an effective target for photothermolysis. Both laser ablation and apoptosis were achieved with a single 50 ns laser pulse with a fluence of only 18 J/cm².

The small particle size enhances transport rates in leaky vasculature in tumors, extracellular fluid, cell membranes, and within cells. It also minimizes rapid clearance by the reticuloendothelial system, particularly in the liver and spleen, especially with the flexible hydrophilic polyvinylalcohol (PVA) coating. The nanorose are multifunctional in that the super-paramagnetic iron oxide cores can serve as contrast enhancement agents for magnetic resonance imaging (MRI). In addition, the relatively non-toxic components, iron oxide, Au, dextran and PVA are potentially acceptable for administration to humans, in contrast with commonly used gold particle stabilizers such as cetyl trimethylammonium bromide.

Nanoroses were formed by the reduction of HAuCl₄ onto the surfaces of 5 nm iron oxide nanoparticles by a reported hydroxylamine seeding procedure, but with several key modifications including the use of a polymeric steric stabilizer, dextran. Previously, ˜60 nm Au-coated magnetic iron oxide nanoparticles were formed with a molar Au:Fe precursor ratio of 2 after the first iteration. In our study, the much smaller Au:Fe ratio 0.1 after all of the iterations led to slower reduction and a relatively open cluster of much smaller primary gold domains, on the order of 8-10 nm (FIGS. 19B and 19C). These domains are easy to discern near the periphery, but are somewhat masked towards the center, where the electrons pass through a much thicker cross-section. The dextran molecules on the iron oxide surface may have helped prevent the gold domains from growing too thick. The gold coatings on iron oxide cores increase the attractive van der Waals forces between particles. The balance of these attractive forces and the steric stabilization provided by dextran, along with the iron oxide and gold precursor concentrations, resulted in kinetic control of the cluster size with the relatively low polydispersity shown by DLS (FIG. 19D). In contrast, aggregation of gold nanoparticles by variation of surface charge in a recent study led to large micron-sized 3-D assemblies with NIR absorption.

Synthesis of dextran coated iron oxide nano-dispersion and purification. The iron oxide nanoparticles were synthesized by using a modified method of Shen. Briefly, 15 ml of Dextran aqueous solution (15% w/w) was titrated with 4 ml NH₄OH (>25% w/w) to pH 11.7. The alkali-treated dextran solution was heated in a flask with magnetic stirring to 25° C. in a water bath. 5 ml fresh prepared 0.75 g FeCl₃.6H₂O and 0.32 g FeCl₂.4H₂O aqueous solution was gradually injected into the alkali-treated dextran solution after passing through a hydrophilic 0.2 μm filter. The black suspension was stirred for a half hour. The subsequent mixture is centrifuged at 10,000 rpm for 20 min. to remove the aggregates. The supernatant was decanted and dialyzed against DI water for 24 hrs. For a dialysis bag with 25 kDalton molecular weight cut off, heavy metal ions, excess salts, ammonium and unbound dextran molecules were removed from the particle dispersion. To concentrate the dispersions and further remove free dextran from the particles, a centrifugal filter device was used in a 1500 rcf speed.

The size of the iron oxide nanoparticles in the end product measured by HRTEM was 5.2±0.8 nm. DLS showed an average hydrodynamic diameter of 12 nm at 25° C. by measuring a diluted iron oxide aqueous solution (0.1 mg/ml Fe). The final colloidal solution had a pH value of 7.3. The concentration of this iron oxide final solution was determined using FAAS and it was found to be 14.6 mg Fe/mL.

Elemental analysis by flame atomic absorption spectrometer. A GBC 908AA flame atomic absorption spectrometer (FAAS) with air/acetylene flame was used for Au and Fe determination. Hollow cathode lamps, gold (Au) and iron (Fe), were operated at the manufacturer recommended current (4 mA for Au and 5 mA for Fe) and the following wavelengths: 242.8 nm for Au and 248.3 nm for Fe. 0.5 ml samples/standards were nebulized into the flame. Six Fe and Au ion standard solutions ranging from 0.5 to 6.0 μg/ml were made for calibration graph. All standards were prepared in 1% nitric acid solution, and same diluent was used as a blank. The linear correlation coefficient is as good as r²=0.9990. The absorption of each sample at the two wavelengths was used to determine the Fe or Au concentration according to the previously prepared standard calibration curve. The observed mass ratio of Au:Fe varied between 3:1 to 4:1.

The Pariss hyperspectral system is coupled to a Leica microscope and measures the spectra of transmitted light at each pixel in an image, for illumination with a halogen lamp (300 to 780 nm). A single vertical section of the sample image is projected onto a prism through a 25 μm slit, and a prism disperses the one-dimensional image onto a two-dimensional Q-imaging Retiga EXi CCD detector, with spatial information encoded on one axis and spectral information on the orthogonal direction. The macrophage samples were laterally scanned via a piezoelectric stage to construct a three-dimensional hyperspectral data cube. A blank slide containing 1×PBS was used to acquire spectrum of the illumination lamp.

FIG. 19E shows an energy dispersive spectroscopy (EDS) area scan coupled with HRTEM from one nanorose. C and Cu peaks are from TEM sample grid (Cu) and ultrathin carbon substrate.

FIG. 19F shows the magnetization vs field strength at 300K. The saturation magnetization of a dried nanorose dispersion at 300 K was 34 emu/g iron oxide (based on Fe₃O₄) as measured by a superconducting quantum interference device magnetometer. The magnetization approached the value of 39 emu/g for the original 5 nm iron oxide nanoparticles, suggesting little interference from the gold coating. To convert from magnetization per total mass of particles to a basis of per mass of Fe₃O₄, the mass ratio of Au:Fe was 3:1 as determined by FAAS, and the polymer amount was 12% as determined from TGA.

FIG. 19G graphs an average optical density spectra v.s. incident light wavelength in macrophages labeled with different nanorose concentrations. The average OD values over 3 to 4 macrophage areas are collected between 460 nm and 800 nm spectra range.

The small hydrodynamic diameter of the nanorose in deionized water of 25 to 35 nm changed relatively little to an average of only 35 nm in 3 months as shown in FIG. 19D indicating effective steric stabilization by the dextran and PVA. According to SEM, the average diameter was approximately 40 nm. The smaller hydrodynamic versus geometric diameter is consistent with a ratio of hydrodynamic radius (DLS) to radius of gyration (static light scattering) of 70-80% from a previous study of open clusters of silica particles of similar size and shape. It is also consistent with the ability of the solvent to flow through spaces and crevices in the open clusters.

Energy dispersive x-ray spectroscopy (EDS) measurements of 20 nanorose particles indicate that the Au-to-Fe molar ratio varied from 5:1 to 8:1. The smaller ratios of 3:1 to 4:1 determined from flame atomic absorption spectroscopy (FAAS) resulted from excess iron oxide particles (without gold shells) in the dispersion, which were seen by TEM (not shown). From the molar ratio determined by EDS and the assumption that the occupied volume within an effective spherical nanorose (with diameter equivalent to the end-to-end distance) was approximately 50% (FIG. 19B), the calculated number of iron oxide particles per nanorose was approximately 100. The number of primary particles in the nanorose in FIG. 19B was of similar order. Thus, most of the 8-10 nm primary particles in FIG. 19B contained a 5 nm iron oxide seed particle in the core. The presence of core-shell primary particles is further supported by the magnetization and an EDS line scan across the particle that shows Fe throughout the particle.

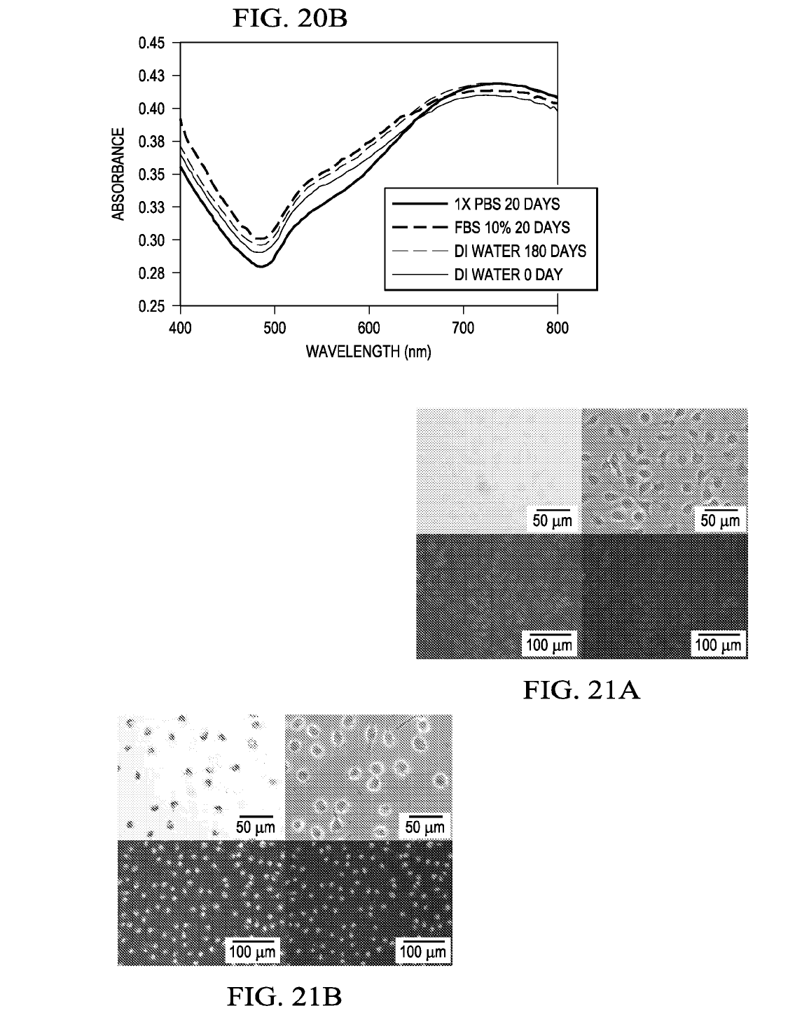

The broad absorbance of a colloidal nanorose dispersion shown in FIG. 20B covers the relevant window for NIR imaging, and drops only 5% at 800 nm from the maximum at 700 nm. The high colloidal and optical stability of the nanorose may be attributed to stabilization against growth or collapse of the gold domains by the iron oxide and polymer stabilizers. For the concentration of gold in the dispersion of 32 μg/ml determined by FAAS in FIG. 20A and an optical path length of 1 cm, the extinction coefficient at the maximum absorbance at 700 nm, ∈₇₀₀=0.025 cm² μg. Assuming that gold occupies ˜50% of the volume of a nanorose (end-end distance) based on FIG. 19, the dispersion in FIG. 20A contained 10¹⁰ nanoroses per ml and thus a particle extinction cross section σ₇₅₅ nm=1×10⁻¹⁴ m². Similar particle cross sections were observed for nanocages, nanorods and nanoshells. The nanorose cross section at 755 nm is 6 orders of magnitude larger than that of freshly prepared indocyanine green dissolved in NaCl aqueous buffer solution (1×10⁻²⁰ m² at 778 nm), which has been used as a NIR dye for laser photothermal therapy to treat cancer.

An examination of the particle shape reveals several reasons for the red shift of the SPR to the NIR region. Various trimers and tetramers of primary particles may be identified in FIG. 19B in the shape of relatively high aspect ratio rods or bent rods containing kinks where the particles touch. The high aspect ratio of these rods shifts the SPR to the red relative to a dense spherical cluster composed of uniformly spaced primary particles. In summary, the strong NIR absorbance may be attributed to a combination of geometric factors: the coupling of the SPR of the primary particles that are in close proximity (FIG. 19B), the high aspect ratio of the small oligomers of the coupled particles in the open cluster (as in nanorods and chains), and thin dimensions (8-10 nm total, 5 nm iron oxide) in certain locations from the surface of an embedded iron oxide particle to the outer edge of the gold shell (as in nanoshell).

The normalized saturation magnetization at 300 K was 34 emu/g iron oxide as measured by a superconducting quantum interference device magnetometer. The magnetization approached the value of 39 emu/g for the original 5 nm iron oxide nanoparticles, suggesting little interference from the gold coating.

Macrophages are implicated in every stage of atherosclerosis from lesion initiation to clinical presentation. Macrophage targeting via administration of NIR sensitive nanoparticles may enhance diagnosis and therapy in situ. Thus, primary mouse peritoneal macrophages were chosen as an in vitro model for cell imaging and photothermolysis.

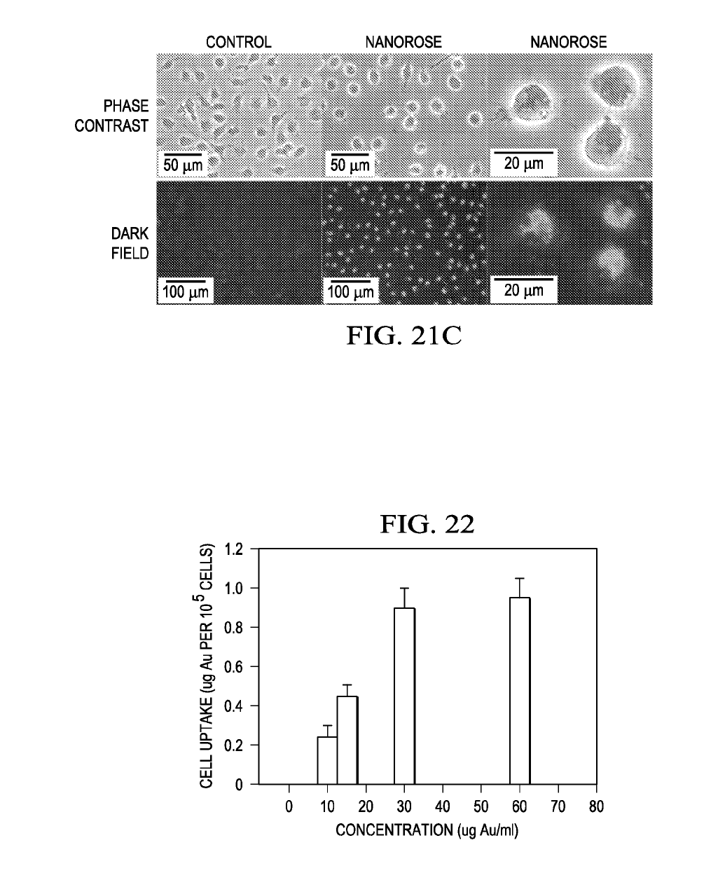

After isolating the peritoneal macrophages and plating them into chamber slides, we prepared various concentrations gold nanorose culture media. We observed macrophage cultures after 24 hours with four different microscopy techniques to confirm nanorose uptake by macrophages (FIG. 21). In bright field transmission mode (40× objective lens), macrophages are nearly transparent due to very weak reflectance and absorption of white light by cytoplasm and cell membrane. In phase contrast mode (40× objective lens) cell membranes are more visible. Similar low contrast images were recorded in dark field reflectance mode (20× objective lens) (FIG. 21A). After 10⁵ macrophages were cultured with 0.1, 1.0, 10, 15, 30 and 60 μg Au/ml nanoroses in DMEM supplemented with 10% FBS media, these nanorose-laden macrophages showed a significant contrast enhancement in all four microscopy modes. In FIG. 21B for 10 μg Au/ml dosage, dark blue color within macrophages in bright field mode indicates absorbance of white light by nanoroses. The brown area within the cell membrane under phase contrast mode shows the configuration of both nanoroses and macrophage membrane clearly. The red color observed in reflectance after passing through a 610 nm long pass filter in dark field mode proved 610 to 800 nm light was strongly reflected by nanorose labeled versus unlabeled native or empty macrophages. FIG. 21C is an image of phase contrast and dark field microscopy images of macrophages labeled with nanorose in DMEM supplemented with 10% FBS media. The left panels do not include nanoroses. The middle and right panels at two different levels of magnification include nanoroses at 10 μg Au/ml. The darkfield reflectance images (20× objective lens) included a 610 nm long pass filter in the path of illumination. All images were recorded with Xe lamp illumination.

A high optical contrast for labeled macrophage cells for a relatively low nanorose dosage requires high cell uptake and a strong absorbance cross section per nanorose cluster. As shown in FIG. 22 for a concentration of only 30 μg Au per ml culture media, the uptake reached saturation at 10⁴ nanoroses per cell for 10⁵ macrophage cells. This uptake level is far above the minimum value of a few hundred required to discriminate between nanorose-labeled versus unlabeled macrophage cells under dark field microscopy with a 40× objective lens. The optical densities, log₁₀ Iₒ/Iₛₐₘₚₗₑ, of nanorose loaded macrophages, were collected with a PARISS hyperspectral imaging instrument in transmission brightfield mode with a halogen illuminator. For the three gold concentrations indicated, 10⁵ macrophages were incubated with nanoroses in DMEM culture medium supplemented with 10% FBS for 24 hours. A pronounced increase in absorbance was observed over this concentration range reaching 0.6 indicating the potential for high contrast in NIR optical imaging despite the relatively low nanorose dosage.

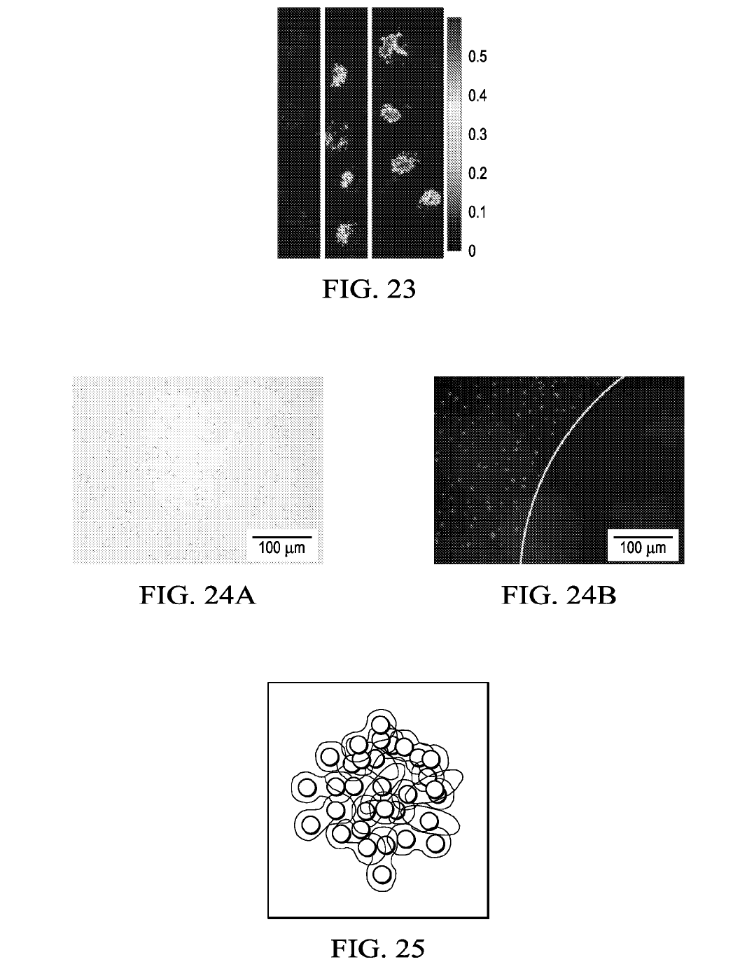

Macrophage cell killing with near infrared pulsed laser and temperature measurement. The high NIR absorption of the nanorose is also beneficial for photothermolysis. In FIG. 23, the macrophages were irradiated with a single 50 ns 18 J/cm² laser pulse emitted from a Q-switched alexandrite laser (50 ns) with a 2 mm diameter spot size. An indium-gallium-arsenide infrared detector was used to measure the temperature after irradiation. Immediately after irradiation, FIG. 23 shows a 0.7° C. increase over the 2 mm spot, indicating strong absorption by the nanorose. Outside the beam, macrophage cells were brown in a bright field image with TUNEL staining demonstrating the ability to achieve apoptosis, which is also of interest in photothermal therapy.

Nanorose growth and purification. 0.1 mL (14.6 mg Fe/ml) 5 nm dextran coated iron oxide nanoparticles were dispersed in 8.9 mL DI water. Dextrose and 100 μL 1% hydroxylamine were added and adsorbed on the surface of iron oxide nanoparticles. The hydroxylamine catalyzed reduction of gold ions on the iron oxide particle surface selectively relative to the free gold ions in solution. Before starting the Au precursor addition, 20 μL of 7% NH₄OH solution was added to tune the pH to be 9.0. An aliquot of 6.348 mM HAuCl₄ aqueous solution was added with at least 10 minutes between each addition. A total of 400 μL of HAuCl₄ were performed. A gradual change in color from brown to dark brown occurred as the precursor addition was increased. The pH gradually decreased to reach a final at 7.0.

The dense gold-coated iron oxide particles were separated from the less dense uncoated particles by centrifugation. After decanting the supernatant, purified gold-coated iron oxide nanorose were redispersed in DI water. Dialysis bags were used to purify the nanoroses further against DI water for 24 hours and the dispersions were sterilized by passage through a 0.45 μm pore size Nylon filter. The purified particles were then concentrated by centrifugal filter devices to 700 μg Au/ml. The final products appeared dark blue in color to the unaided eye. To improve the steric stabilization of the nanorose clusters, poly (vinyl alcohol) (PVA) MW 22,000, was added into the dispersions. After 3 months storage, a small portion of the settled particles were re-dispersible by manual shaking without any visible clusters. After washing the nanoroses twice with DI water followed by centrifugation at 8000 rpm and drying, thermogravimetric analysis (TGA) indicated that the concentration of polymer was 13% (w/w).

Dynamic light scattering analysis was performed in triplicate on a custom-built apparatus (scattering angle: 90°) and the data were analyzed using a digital autocorrelator and a non-negative least-squares (NNLS) routine. The dispersion concentration was ˜0.02-0.04 mg/mL which gave a measured count rate of approximately 300-400 kcps. All dispersions were filtered through a 0.2 μm filter and probe sonicated for 2 min prior to measurement.

FIG. 24 shows the laser ablation of macrophage cells in vitro with a single 50 ns pulse under a fluence of 18 J/cm². FIG. 24A, after irradiation without nanorose, the bright field image with TUNEL staining indicates the macrophage membranes were intact. FIG. 24B, A dark field image shows interaction of the laser beam with the nanorose in the irradiated area vaporized the macrophage cells. FIG. 24C, Temperature profile over the 2 mm diameter irradiated area. FIG. 25 is a schematic of nanocluster of gold coated iron oxide primary particles, the lines show the gold shell domains.

Macrophage cell culture. Peritoneal macrophages were isolated from C57BLKS mice to demonstrate the targeted uptake of nanoroses and microscopic imaging enhancement. The macrophages were cultured on chamber slides in phenol-free DMEM plus 10% FBS media at 37° C. in 5% CO₂ for 24 hours before they were treated with nanoroses. The nanorose suspensions at different gold concentrations were mixed with the cell culture media immediately prior to addition to isolated macrophages. 1 mL of nanorose medium was incubated in each chamber for 24 hours to maximize uptake by macrophages before an intensive 1×PBS washing. The non-engulfed nanoparticles were removed from the chamber prior to elemental analysis of metals by FAAS. The laser treatments were performed on these same chamber slides while they were covered to minimize contamination. The macrophages were cultured for another 24 hours after each laser treatment before staining or microscopy imaging.

Macrophage photothermal treatment and infrared detector setup for temperature measurement in vitro. The macrophage culture slides had two chambers. One chamber was filled with a monolayer of macrophages which had engulfed nanoroses. The nanorose concentration was maintained at 1 μg/ml of gold. The second chamber was filled with a monolayer of non-labeled macrophages only, which was used as a control. The nanorose treated macrophages were irradiated with a single 755 nm pulse of 50 ns duration and 2 mm spot size providing a fluence of 18 J/cm². 8 spots per chamber were pursued to show the reproducibility. The control was irradiated with the same specification laser dosage under the same procedure.

A Candela ALEXLAZR© at wavelength of 755 nm with adjustable fluence was used to irradiate macrophages in vitro on the chambered slides. An Indium-Gallium-Arsenide (InGaAs ranging from 1.0-2.4 Microns wavelength) infrared detector was used to measure the temperature when the macrophages were irradiated. The laser radiation was angled onto the macrophages so that the detector would not capture the laser beam but capture only the IR radiation from the heating effect caused by the laser. The infrared emission from the macrophages was focused by a 25 mm focal length Calcium Fluoride lens onto a parabolic mirror with 3.5 cm focus. The IR reflected from the parabolic mirror was focused onto the InGaAs detector. The InGaAs detector was connected to an amplifier to convert the detector output current to a voltage. A data acquisition (DAQ) card was then used to capture the voltage value. An automated LABVIEW© visual interface was used to record the temperature data for a period of 10 seconds. Temperature calibration was performed using a black body radiator.

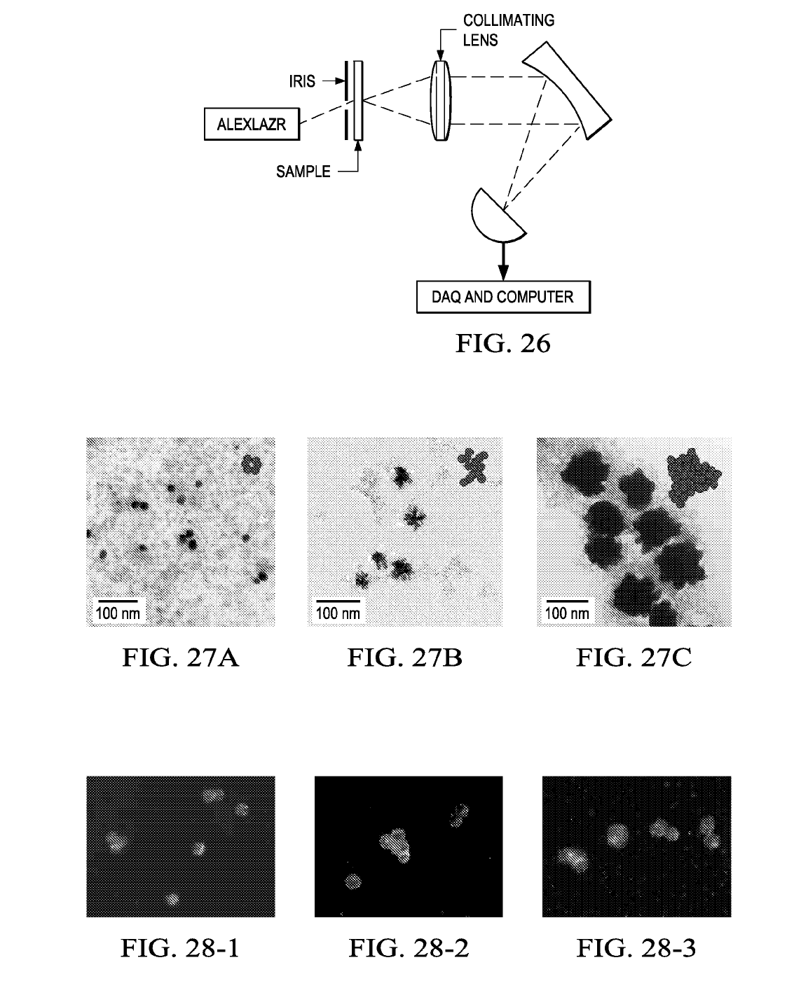

FIG. 26 shows the apparatus for taking an infrared temperature measurement using HgCdTe single point detector (Fermionics Corp Model #PV-11-1) and the temperature profile. The 755 nm laser (Alexlazr) was at an angle of 45° to prevent the incident beam from being sensed directly by the IR detector.

Design of size and shape of hybrid magnetic/plasmonic nanoclusters size to enhance the therapeutic effect. Nanoclusters have been designed with controlled size, curvature and shape to enhance the therapeutic effect of the conjugated biomolecules. When diameters of Ab coated gold nanospheres are reduced to 20 to 50 nm, the biological pathways in targeted cells can undergo profound changes. The nanoparticles serve not merely as substrates for the Abs but strongly influence the effect of the Abs on the biological signaling processes. The fact that the curvature of the gold nanospheres influence binding capacities by nearly 3 orders of magnitude suggests that interactions between multiple Abs on the surface and cell receptors play a key role. In addition to nanoparticle size, cell targeting is influenced by particle shape, and recent studies have investigated ellipsoids, rods, cylinders and disks in addition to spheres. The goal is to be able to control the size, shape and curvature of the nanoparticle, and to conjugate multiple Abs onto the particle surface for enhanced targeting to advance imaging and therapy.

The nanocluster assembly platform of the present invention is highly flexible and robust for controlling both the curvature of the gold shells on the primary particles and the size of the clusters. Furthermore, the presence of gold shells on the clusters provides a general surface for conjugation of multiple targeting and therapeutic moieties. This approach is applicable to the biodegradable nanoclusters, including the gold nanoclusters, and the nanorose iron/oxide gold nanocomposites. These morphologies have been achieved by changing the gold to iron oxide ratio as shown in FIGS. 27A and 27C, both above and below the values for our standard nanoclusters in FIG. 27B. As this ratio increases the thicker gold shells leads to a greater degree of clustering of the primary particles. The size of the gold-coated primary magnetic particles may be varied from 3 nm to 8 nm to change their curvature. In addition, the size of biodegradable nanoclusters, including the gold nanoclusters, and the surface properties of the nanoclusters may be varied to influence the shape and curvature. With this robust approach, the spacings of the Abs on the gold shells on the individual primary particles and on adjacent shells may be varied over a wide range. This approach offers vast new opportunities for therapeutic enhancement from multiple interactions between Abs on surfaces of varying curvatures and cell receptors. For example, the nanoclusters begin to mimic the size of viral capsids (nanoclusters of proteins), which when labeled with antibodies, provide highly effective cell targeting of the transmembrane protein tyrosine kinase 7 receptors on leukemia cells. The curvature of the primary particles (˜5 nm) is similar to that of the protein spheres that make up viral capsids. Furthermore, the surface of the primary particles on the gold nanocluster surface has been conjugated with EGFR to selectively target cancer cells as was shown for one particular nanocluster geometry.

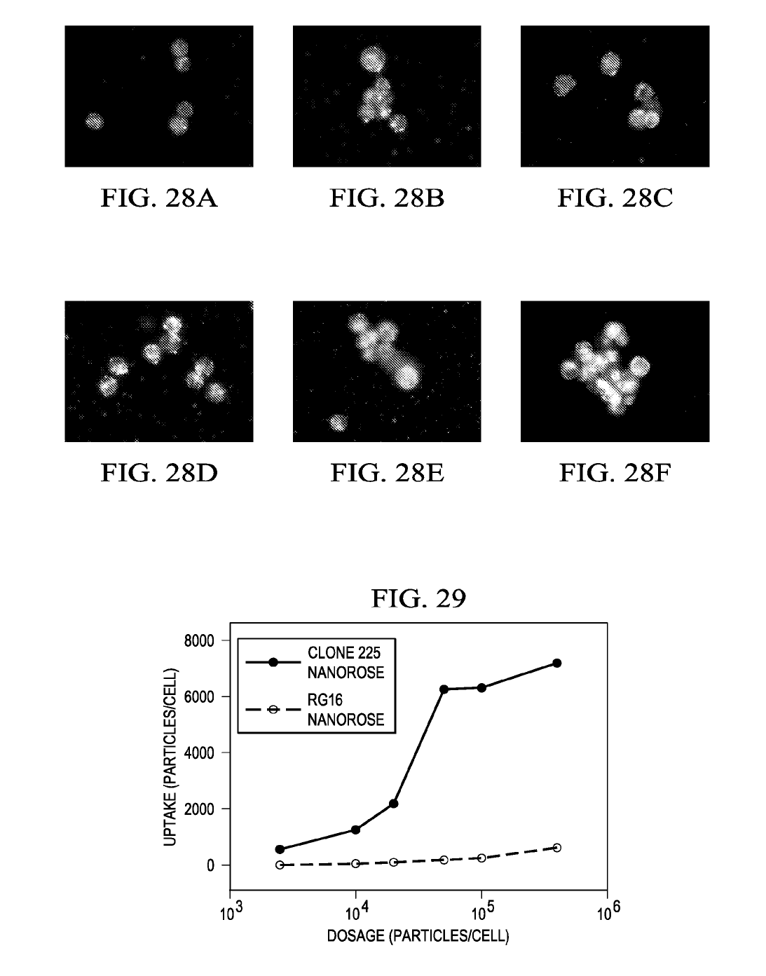

Anti-EGFR Neomarker clone 225 antibodies were purified using a 100 k MW filter from Centricon and then mixed with 0.1 M sodium periodate. This results in oxidation of carbohydrate moieties on the antibody's Fc region to aldehydes. The reaction was quenched with phosphate buffered saline (PBS) and then a hydrazide polyethylene glycol-thiol heterobifunctional linker molecule was mixed with the antibodies for 20 min. During this step the hydrazide portion of the polyethylene glycol (PEG) linker interacts with aldehyde groups on the antibodies to form a covalent bond. One more filtration step was used to remove excess linker molecules. The antibody/linker solution was diluted in the organic buffer 4-(2-hydroxyethyl)-1-piperazineethanesulfonic acid (HEPES), pH 8.75, to 0.05 mg ml⁻¹ and mixed with the gold/iron oxide particle solution in a 1:1 volume ratio for particle functionalization via gold-thiol interactions. The mixture was agitated for 30 min at room temperature and then a small amount of 10⁻⁵ M 5 kD mPEG-thiol was added to coat any remaining bare gold surface. After thirty minutes 2% 18 kD PEG in PBS was added and the particles were centrifuged at 6000 rpm for 10 min and resuspended in 1% PEG in 1×PBS. FIG. 28 are dark field microscopy images of A431 skin cancer cells cultured with different dosage Clone 225 conjugated nanoroses for 1 hr. a, b, c, d, e and f represent typical images of treated cells with dosage 2.5×10³, 1.0×10⁴, 2.0×10⁴, 5.0×10⁴, 1.0×10⁵, 4.0×10⁵ nanoroses/cell. Control experiments were done by using rabbit IgG antibody (RG16) conjugated nanoroses to tread A431 cells in the same conditions with dosage (1) Non-treated A431 cells, (2) 2.0×10⁴ RG16 nanoroses/cell, (3) 4.0×10⁵ RG16 nanoroses/cell. The dosage response of A431 cells targeted with Anti-EGFR C225-conjugated nanoroses was studied by dark field microscopy and atomic absorbance spectroscopy. As shown in FIG. aa, the orange color density within cells increased as dosage of C225-conjugated nanoroses increased from 2.5×10³ to 4.0×10⁵ nanoroses/cell, which was correlated to the particle density increase. Control experiments were done by using a general rabbit IgG antibody (RG16) which was conjugated to nanoroses to treat the A431 cells under the same incubation conditions. The invisible orange color inside cells suggested a much weaker binding strength between particles and cells. Therefore, a high selectivity of targeting can be realized by conjugation of specific antibody (C225) to nanoroses. The results were further quantified with atomic absorbance spectroscopy. The element gold was chosen to identify the quantity of nanoroses which were bound to cells. As shown in FIG. bb, an average of 500 to 7000 C225 nanorose conjugates were detected within one cell according to a dosage increase from 2.5×10³ to 4.0×10⁵ nanoroses/cell. In contrast, a lower number of cells was detected for the non-specific RG16 nanorose conjugates. The high selectivity confirmed the effectiveness of the selective targeting of these antibody-nanorose conjugates to the receptors on the surface of cancer cells.

FIG. 29 is an image of cell uptake dosage response of clone 225 and RG16 conjugated nanoroses. 10⁵ to 10⁶ A431 cells were incubated with dosage 2.5×10³, 1.0×10⁴, 2.0×10⁴, 5.0×10⁴, 1.0×10⁵, 4.0×10⁵ nanoroses/cell for 1 hr. The particle targeted cells were separated from free nanoroses by centrifugation at 1000 rpm for 3 mins. After repeating centrifugation twice, the precipitates were dissolved in 0.5 ml 1 mM HNO₃ for AA elemental analysis.

The nanorose conjugates were characterized by UV-Vis spectroscopy, SEM, TEM, and DLS. Various amounts of clone 225 antibodies were conjugated to nanoclusters, and the hydrodynamic diameters were measured by DLS. For the lowest ratio C225/Au ratio of 5/40 by weight, the average diameter was 35 nm, only about 20% larger than the original unconjugated particles. Even at this low ratio, high targeting efficiency was observed by DF microscopy. The nanocluster sizes were also acceptable and below 60 nm at a much higher ratio of 125:40 illustrating the high colloidal stability of the conjugated nanoclusters.

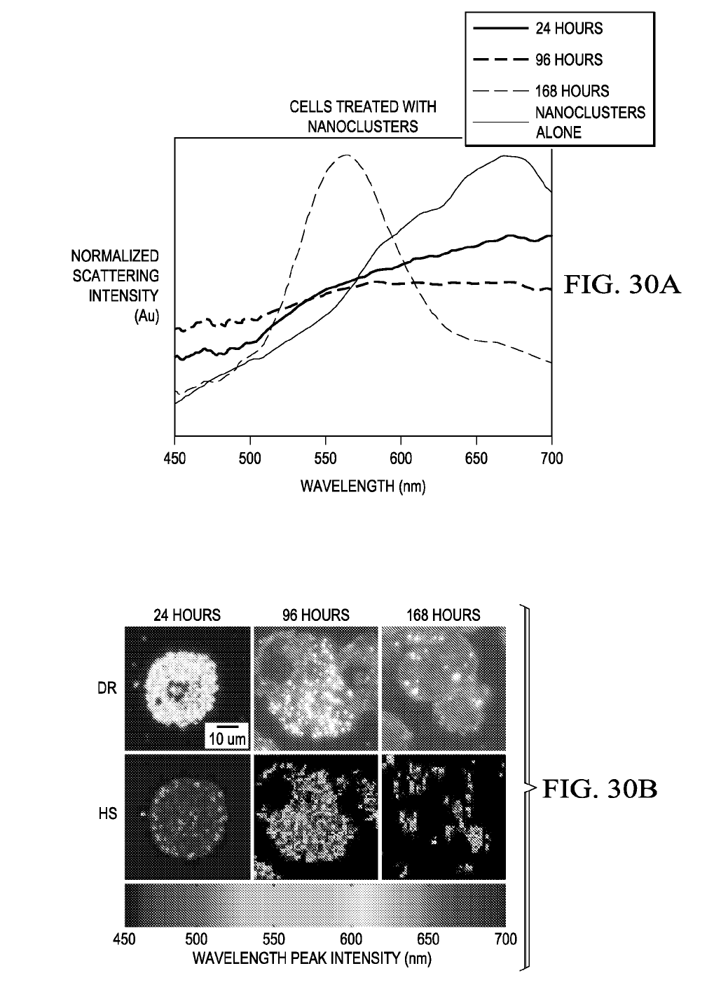

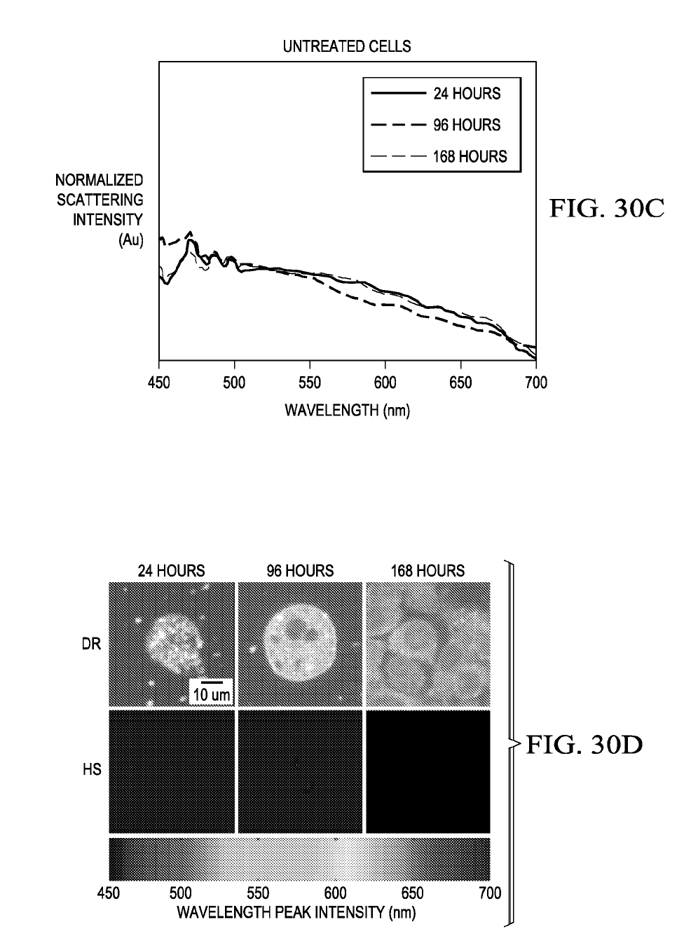

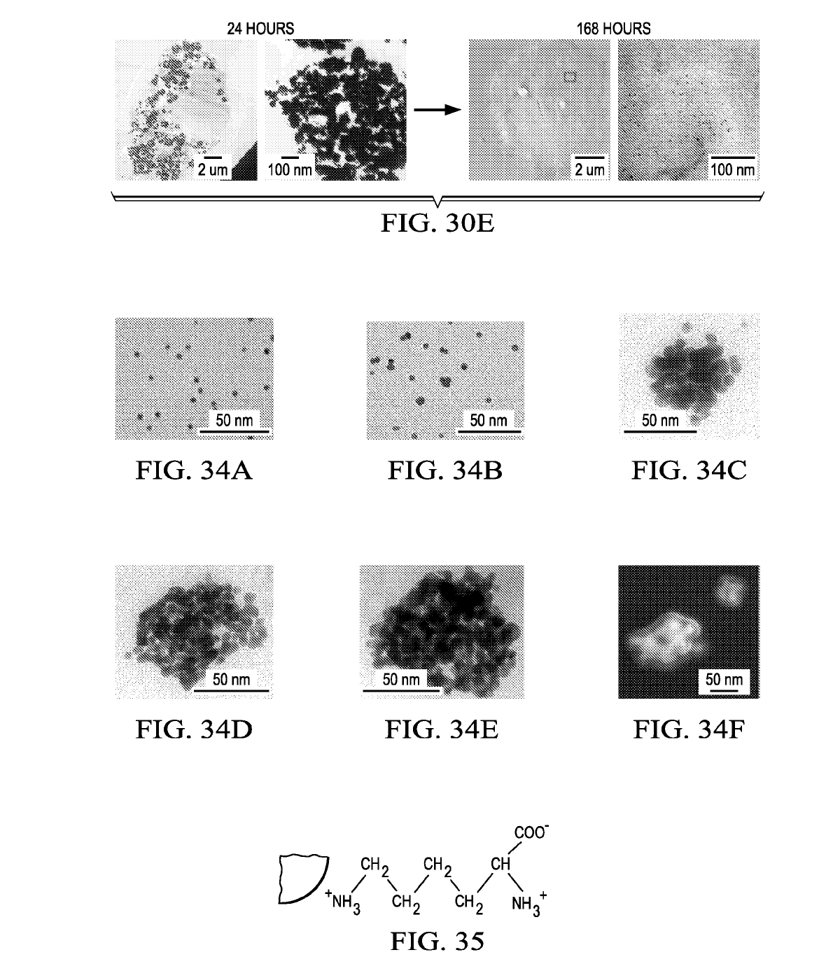

FIGS. 30A-30E are images of the biodegradation of gold nanoclusters inside live cells. Scattering spectra (normalized by area under the curve) of FIG. 30A live cells labeled with nanoclusters and (FIG. 30C) control cells without nanoclusters. The spectra were taken at 24, 96, and 168 hours time points after cells were treated with nanoclusters. Dark-field reflectance (DR) images of cells treated with (FIG. 30B) nanoclusters and (FIG. 30D) control cells over time are shown together with corresponding color coded images indicating scattering peak position at each pixel in the field of view (FIG. 30B and FIG. 30D, bottom rows). Hyperspectral (HS) images and the color bar were used to obtain the color coded distribution of scattering peaks within cells (FIG. 30B and FIG. 30D, bottom row). Pixels that did not have an identifiable peak in a corresponding spectrum were not assigned a color. The scale bar in the DR and HS images is 10 μm. (FIG. 30E) TEM images of cells treated with nanoclusters at low magnification (scale bar 2 um) and high magnification (scale bar 100 nm) at 24 hours and 168 hours. Red boxes in the low magnification images are magnified on the right at each time point.

Scattering spectra from hypespectral images of cells (FIGS. 30A and 30C), dark-field reflectance (DR) (FIG. 30B, 30D, top row), and hyperspectral (HS) images (FIGS. 30B and 30D, bottom row) were acquired at 24, 96, and 168 hours time points after cells were treated with nanoclusters. High nanocluster uptake was evident in the DR images, where nanoclusters strongly scattered illumination light; overall scattering intensity decreased over time as macrophages divided and nanoclusters were distributed between daughter cells (FIG. 30B, top row). A significant increase in the red-NIR scattering signal of the labeled cells was seen compared to unlabeled cells (compare FIG. 30A, dark blue curve, and FIG. 30C), consistent with the high scattering efficiency of the nanoclusters in solution (FIG. 30A, light blue curve). The relative intensity of the red-NIR scattering signal decreased after 96 hours and the scattering from labeled cells showed a marked blue shift to ˜550 nm that is consistent with scattering from the constituent lysine/citrate-capped gold nanoparticles. Hyperspectral images showed a gradual progression from very strong scattering in the 650-700 nm region at t=24 hours to a less intense scattering signal predominantly in the 500-550 nm region at t=168 hours (FIG. 30B, bottom row). The scattering for the control cells did not significantly change with time.

The biodegradation of nanoclusters inside live cells was further confirmed by TEM (FIG. 30E). Approximately 3×10⁴ macrophage cells were seeded overnight on Aclar Embedding Film. All samples for TEM imaging were treated identically and were run in parallel to the samples used for optical imaging. At each time point, the cells were fixed in a 1% glutaraldehyde and 1% paraformaldehyde solution for 1 hour at room temperature and then washed 3 times in PBS. Subsequently, cells were stained with 2% osmium tetroxide in water for 10 minutes and washed for 10 minutes in water. The sample was then dehydrated using increasing ratios of ethanol to acetone solutions, and finally embedded in an epoxy-acetone mixture and allowed to bake at 60° C. for 24 hours. Ultrathin sections were sliced using a Leica Ultracut microtome, and imaged with the Tecnai G2 TEM at a voltage of 80 kV. After 24 hours, large ˜100 nm nanoclusters can be observed throughout the interior of cells (FIG. 30E, 24 hours), whereas after 168 hours, cells contain only particles less than 5 nm in diameter (FIG. 30E, 168 hours). These TEM results are in excellent agreement with optical measurements and with deaggregation results in solution, providing unambiguous proof of essentially complete biodegradation of the initial ˜100 nm nanoclusters into sub-5 nm primary particles.

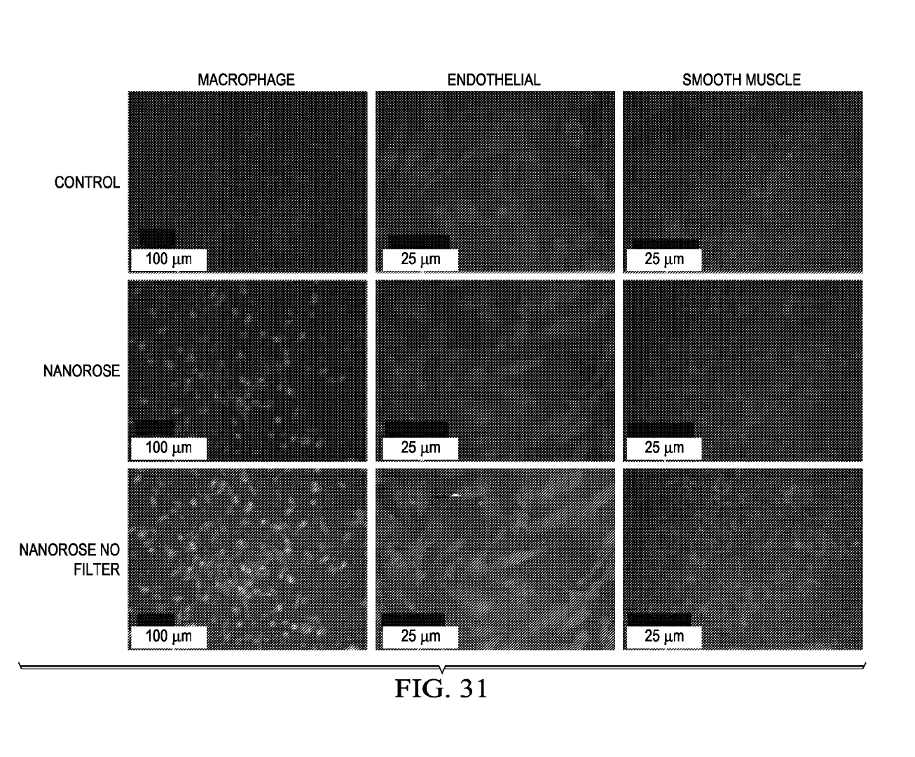

FIG. 31 is an image of the specificity of nanorose uptake into peritoneal macrophages versus aortic endothelial cells and aortic smooth muscle cells by dark field microscopy with a 610 nm long pass filter. The top row is a control without incubation of nanorose. In the middle row, the bright spots indicate NIR reflectance from nanorose in macrophage cells, at wavelengths above 610 nm, which is not evident for the other cells. The lower row shows the cellular morphology in greater detail by dark field imaging without a filter.

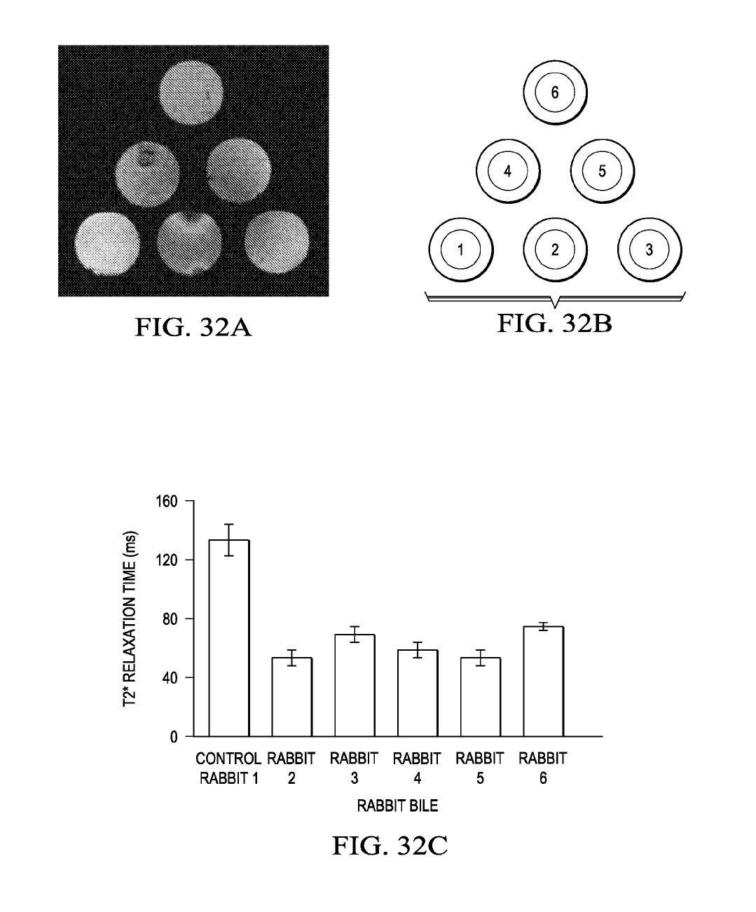

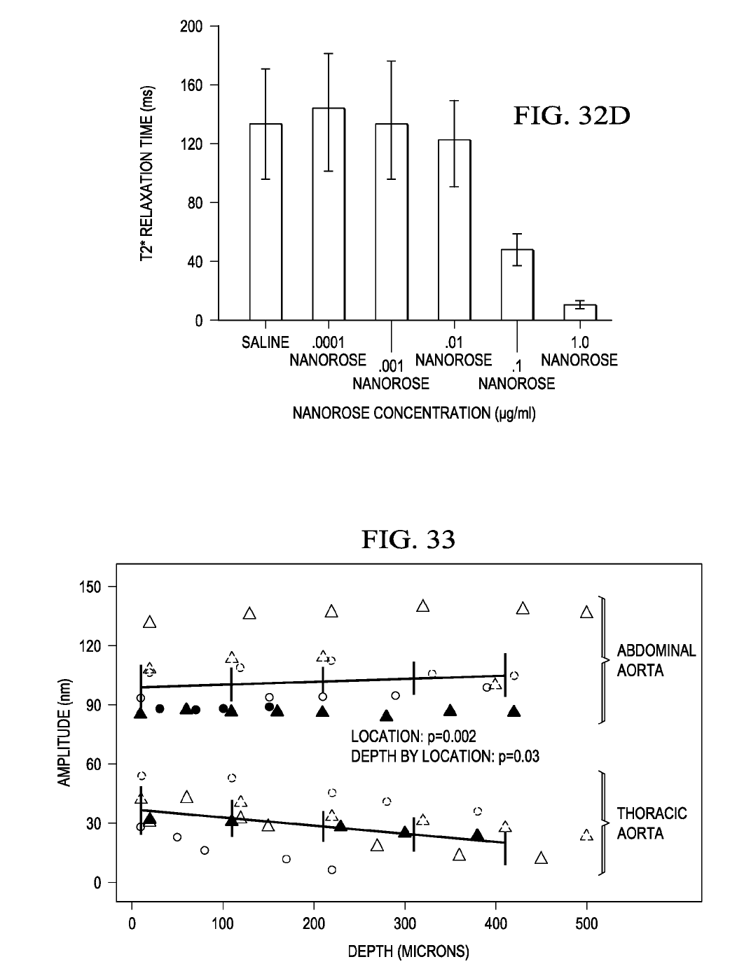

FIG. 32 is a evidence of nanorose excretion via bile detected with 7T MRI. Due to the iron oxide core of each “nanopetal”, and the open design of the nanorose which allows a large surface area for interaction with protons (water), the nanorose have a stronger MRI signal than FDA approved FERRIDEX®. Atherosclerotic rabbits were iv injected with nanorose (1.4 mg nanorose Au/kg rabbit body weight, n=5) or saline (n=1), and 3 days later sacrificed for collection of bile and urine. A. The raw MRI image of tubes containing bile from each rabbit is shown. A spoiled multi-echo gradient sequence, with 12 echos spaced 3.85 msec apart (TR=1500 ms) was used and the 12th echo image displayed. The darker the image, the more iron oxide present. As is visibly evident, the bile from the iv nanorose injected rabbits is darker than the control bile, consistent with nanorose excretion via the reticulo-endothelial system. FIG. 32B is a cartoon demonstrating the locations of regions of interest (ROI) analyzed to avoid artifacts in the samples. The iv nanorose rabbits are identified in blue, and the control rabbit with no coloration. FIG. 32C is an image that measurement of the T2* relaxation times from samples shown in A. Standard error bars represent ±95% confidence intervals of the non linear least square fit derived for T2*. FIG. 32D. Standard curve for T2* relaxation times from known concentrations of nanorose in saline (triplicate). Based upon this standard curve, the concentration of nanorose in bile was approximately 0.1 μg/ml, while no paramagnetic signal was present in urine collected from these same rabbits (saline injected rabbit urine T2* 252 msec, vs 247±80 msec in the nanorose injected rabbit urines).

To demonstrate the clinical applicability of nanorose, it is important to demonstrate that the reticulo-endothelial system is able to metabolize and excrete these nanoparticles. Preliminary studies have demonstrated that in acid pH of 4.5, the nanorose lose their NIR tuning and change their color in visible light from blue to red over 3 days, consistent with degradation of the nanorose into nanopetals (data not shown). Since macrophage lysosomes have a pH of 4 to 5, the principal investigator and colleagues hypothesized that if nanorose were incubated in vitro in rabbit macrophage cultures, they would also lose their NIR tuning Macrophages which had engulfed nanorose (1 μg Au/ml), were excited with light between 200-800 nm. The absorbance spectra at 755 nm were collected with a PARISS hyperspectral imaging device, and were consistent with nanorose maximally absorbing light at 755 nm even after being engulfed by macrophages at the 24 hour time point. However, by 3 days, the NIR absorbance spectra were lost, consistent with breakdown of nanorose (data not shown). Based upon these studies, we hypothesized that if nanorose were iv injected into rabbits, by the day 3 time point, paramagnetic activity consistent with the iron oxide core of each “nanopetal” would be evident in the bile, as demonstrated in FIG. 32.

Table 1 below illustrates Chemistry (Chem and LFTs), hematology (CBC) and urine analysis (UA) demonstrating 3 days following the iv injection of 1.4 mg nanorose Au/kg body weight, there was no allergic reaction, and no renal or hepatic toxicity in double balloon injured fat feed New Zealand white rabbits (n=5, p=NS all comparisons).

TABLE 1

| | | Baseline n = 5 | Nanorose n = 5 |
|---|---|---|---|
| Chem | Na (mmol/L) | 141 ± 0.3 | 138 ± 6 |
| | K (mmol/L) | 4.3 ± 0.3 | 3.6 ± 0.4 |
| | Glucose | 124 ± 31 | 122 ± 34 |
| | (mg/dL) | | |
| | BUN (mg/dL) | 14 ± 3 | 13 ± 1 |
| | Creat (mg/dL) | 1.0 ± 0.2 | 1.0 ± 0.2 |
| LFTs | Alk phos (U/L) | 98 ± 37 | 86 ± 29 |
| | Billi total | 0.3 ± 0.1 | 0.3 ± 0.1 |
| | (mg/dL) | | |
| | ALT (U/L) | 42 ± 18 | 44 ± 17 |
| | Amylase (U/L) | 226 ± 61 | 206 ± 51 |
| CBC | Hct (%) | 33.0 ± 5.7 | 25.5 ± 2.1 |
| | WBC (109/L) | 11.3 ± 3.4 | 6.2 ± 2.0 |
| | Eosino (%) | 0.5 ± 0.7 | 1.5 ± 0.7 |
| | Plat (109/L) | 354 ± 76 | 414 ± 205 |
| UA | pH | 8.5 ± 0.1 | 6.7 ± 0.3 |
| | SG | 1.02 ± 0.01 | 1.02 ± 0.01 |
| | Protein | negative | negative |
| | Casts | negative | negative |

FIG. 33 is a graph that replicate amplitude (nm) and depth (microns) measurements in rabbits (n=3) measured in macrophage rich abdominal aorta, and macrophage poor thoracic aorta at up to 6 different depths. The three rabbits are identified by color (blue, green, orange) and paired samples from a single rabbit by symbol (triangle, dot). Statistical testing was two-sided with a significance level of 5% and predicted values were estimated based on a repeated measures linear model in terms of location and depth. For each anatomical location, whiskers extend to the predicted value plus or minus one standard error (Abdomen: Black, Thoracic: Red). The location effect (p=0.002) and the depth by location interaction (p=0.03) were significant indicating variation with mean amplitude (abdominal vs thoracic aorta location) and the slope (relating amplitude and depth, where the signal is stronger in depth in the abdominal aorta, and weaker in depth in the thoracic aorta). These results are consistent with our ability to identify ex vivo plaque based macrophages in atherosclerotic aortic tissue following iv injection of nanorose.

Clusters of metal nanoparticles with an overall size less than 100 nm and high metal loadings for strong optical functionality, are of interest in various fields including microelectronics, sensors, optoelectronics and biomedical imaging and therapeutics. Herein we assemble ˜5 nm gold particles into clusters with controlled size, as small as 30 nm and up to 100 nm, which contain only small amounts of polymeric stabilizers. The assembly is kinetically controlled with weakly adsorbing polymers, PLA(2K)-b-PEG(10K)-b-PLA(2K) or PEG (MW=3350), by manipulating electrostatic, van der Waals (VDW), steric, and depletion forces. The cluster size and optical properties are tuned as a function of particle volume fractions and polymer/gold ratios to modulate the interparticle interactions. The close spacing between the constituent gold nanoparticles and high gold loadings (80-85% w/w gold) produce a strong absorbance cross section of ˜9×10⁻¹⁵ m2 in the NIR at 700 nm. This morphology results from VDW and depletion attractive interactions that exclude the weakly adsorbed polymeric stabilizer from the cluster interior. The generality of this kinetic assembly platform is demonstrated for gold nanoparticles with a range of surface charges from highly negative to neutral, with the two different polymers.

Metal nanoparticles with high NIR absorbance are of great interest in biomedical imaging and therapy because soft tissues and water are relatively transparent from 650 to 900 nm. The surface plasmon resonance (SPR) of a spherical gold particle exhibits a maximum at 530 nm, but undergoes a red shift to the NIR for particles with a hollow or non-spherical geometry, such as nanoshells, nanorods, and nanocages. These particles are typically 50-100 nm in diameter. NIR absorbance has rarely been achieved for particles smaller than 50 nm, where it becomes challenging to synthesize the types of asymmetric morphologies needed for strong red-shifts. Significant NIR absorbance also has been demonstrated in vitro and in vivo for the assembly of 40 nm gold spheres, conjugated with antibodies, by receptors in cancer cells into clusters. Small gold clusters that have been formed by equilibrium self assembly methods often contain high concentrations of templating agents, which result in particle separations greater than one particle diameter and thus small red shifts.

Nanoparticle components may be assembled into clusters with properties that are challenging to achieve including, sizes below 50 nm strong optical absorbance, multifunctionality, and/or biodegradability. Recently, there has been great interest in the development of sub-30 nm particles, which penetrate cell membranes and leaky vasculature in cancerous tumors more efficiently than particles >50 nm. Furthermore, these small nanoparticles elicit profound changes in biological pathways in targeted cells. Sub-30 nm particles have been reported for gold nanocages and multifunctional nanocluster hybrids containing gold and iron oxide, referred to as nanoroses. Despite their small sizes, both types of nanoparticles absorb strongly in the NIR. The nanorose clusters, composed of nanocomposite primary particles, are formed by kinetic assembly during the reduction of gold precursors onto iron oxide nanoparticles. They exhibit intense magnetic relaxivity as well as NIR absorbance. To further advance the functional properties in nanoclusters, especially biodegradability, we recently introduced a physical, rather than chemical, method for the kinetically controlled colloidal assembly of ˜5 nm gold spheres into ˜100 nm NIR plasmonic clusters stabilized by PLA(2K)-b-PEG(10K)-b-PLA(2K). These clusters were shown to biodegrade nearly completely in solution and in macrophage cells back to the original 5 nm primary spheres, which are small enough for renal clearance. This physical, kinetic, colloidal assembly method is general and likely to enable synthesis of many types of clusters over a wide size range.

Herein we assemble kinetically sub-5 nm gold particles into clusters of controlled sizes, as small as 30 nm and up to 100 nm, stabilized by small amounts of a weakly adsorbing polymer, either PLA-b-PEG-b-PLA or PEG 3350. The physical cluster assembly process is illustrated in FIG. 1B. The gold nanoparticles are nucleated rapidly at high volume fractions in the presence of a weakly adsorbing polymer to form small cluster. The nucleation and growth of the gold clusters is controlled by increasing gold and polymer concentrations simultaneously, either by solvent evaporation or by mixing of a concentrated gold dispersion with a concentrated polymer solution. A mechanism is presented to describe the cluster growth and gold particle spacing in terms of the electrostatic, VDW, steric and depletion forces. The combination of high gold particle volume fractions and exclusion of the weakly adsorbed polymeric stabilizer from the cluster interior towards the exterior surface are utilized to produce low polymer loadings and closely spaced gold particles for strong NIR absorbance. In contrast, high polymer loadings and larger gold particle spacings are typically obtained in equilibrium assembly processes that rely on strong interactions with templating agents, such as micelles. Finally, the small amount of polymer on the exterior surface provides sufficient steric stabilization to prevent unregulated cluster growth, in contrast with previous studies without polymer stabilizers. Relative to our previous study, the size of the clusters is over three fold smaller, and furthermore, a wider range of ligands (to modify particle charge), polymers, and polymer/gold ratios are examined. An advantage of this kinetic assembly approach is its use of readily available polymer stabilizers and simple ligands on the gold surface, such as citrate and lysine, in contrast to templating agents that often require complicated synthetic approaches.

HAuCl₄.3H₂O was purchased from MP Biomedicals LLC (Solon, Ohio) and Na₃C₃H₅O(COO)₃.2 H₂O and NaBH₄ were acquired from Fisher Scientific (Fair Lawn, N.J.). L(+)-lysine was obtained from Acros Chemicals (Morris Plains, N.J.). PEG (MW=3350) was ordered from Union Carbide (Danbury, Conn.) and PLA(2K)-b-PEG(10K)-b-PLA(2K) was purchased from Sigma Aldrich (St. Louis, Mo.).

Nanocluster formation Gold nanoparticles (3.8-nm) stabilized with citrate ligands were synthesized based on a well known method. Briefly, DI water (100 mL) was heated to 97° C. While stirring, 1 mL of 1% HAuCL₄.3H₂O, 1 mL of 1% Na₃C₃H₅O(COO)₃.2H₂O, and 1 mL of 0.075% NaBH₄ in a 1% Na₃C₃H₅O(COO)₃.2H₂O solution were added in 1 minute intervals. The solution was stirred for 5 minutes and then removed to an ice bath to cool to room temperature. The gold particles were then centrifuged at 10,000 rpm for 10 minutes at 4° C. to remove any large aggregates. Centrifugal filter devices were used to removed unadsorbed citrate ligands as well as concentrate the gold dispersion to ˜3.0 mg Au/mL. Gold concentrations were determined using flame atomic absorption spectroscopy (FAAS).

In most cases, lysine ligands were added to the citrate stabilized gold nanoparticles by adding a 1% lysine in pH 8.4 phosphate buffer (10 mM) solution to 1.2 mL of the colloidal citrate-capped gold solution to yield a final lysine and gold concentration of 0.4 mg/mL and 3.0 mg/mL, respectively. In the cases where a 1.0 mg/mL gold solution was used to produce nanoclusters, the 3.0 mg/mL stock gold solution was diluted using deionized (DI) water. The dispersion was stirred for at least 12 hours. PLA-b-PEG-b-PLA was added to the aqueous dispersion of ligand capped gold nanoparticles to yield polymer/gold ratios ranging from 1/10-40/1. The dispersions were then sonicated in a bath sonicator for 5 minutes. Unless otherwise noted, the concentration of the gold solutions used in this study to produce nanoclusters was 3.0 mg/mL with a polymer/gold ratio of 16/1.

In some cases, the polymer/gold dispersion was placed under an air stream and a certain percentage of the solvent, between 50-100%, was evaporated. When the dispersion was not dried to completion, it was quenched with DI water after the chosen amount of solvent evaporation. Upon quenching, the concentration of the dispersion was approximately an order of magnitude lower than that of the original gold stock prior to solvent evaporation. In the case of 100% solvent evaporation, which took place over ˜20-30 minutes, the dried film was redispersed with 10 mL of DI water to yield a blue dispersion of ˜0.30 mg Au/mL. Nanoclusters were also formed using a mixing procedure, in which highly concentrated solutions of gold colloid and polymer were mixed together using a probe sonicator (Branson Sonifier 450, Branson Ultrasonics Corporation, Danbury, Conn.) with a 102 converter and tip operated in pulse mode at 35 W.

Nanocluster morphology was observed by transmission electron (TEM) and scanning electron microscopy (SEM). TEM was performed on a FEI TECNAI G2 F20 X-TWIN TEM using a high-angle annular dark field detector. TEM samples were prepared using a “flash-freezing” technique, in which a 200 mesh carbon-coated copper TEM grid was cooled using liquid nitrogen and then dipped into a dilute aqueous nanocluster dispersion. The TEM grid was dried using a Virtis Advantage Tray Lyophilizer (Virtis Company, Gardiner, N.Y.) with 2 hours of primary drying at −40° C. followed by a 12 hour ramp to +25° C. and then 2 hours of secondary drying at 25° C. Separation distances between primary particles within the nanoclusters were measured by analyzing TEM images using Scion Image software (Frederick, Md.). A Zeiss Supra 40VP field emission SEM was operated at an accelerating voltage of 5-10 kV. SEM samples were prepared by depositing a dilute aqueous dispersion of the nanoclusters onto a silicon wafer. The sample was dried in a hood, washed with DI water to remove excess polymer, and dried again. UV-visible spectra were measured using a Varian Cary 5000 spectrophotometer for a 1 cm path length. Dynamic light scattering (DLS) measurements of hydrodynamic diameter and zeta potential measurements were performed in triplicate on a Brookhaven Instruments ZetaPlus dynamic light scattering apparatus at a scattering angle of 90° and temperature of 25° C. Dispersion concentrations were adjusted with either DI water for DLS measurements or pH=7.4 buffer (10 mM) for zeta potential measurements to give a measured count rate between 300-400 kcps. For DLS measurements, all dispersions were filtered through a 0.2 μm filter and probe sonicated for 2 min prior to measurement. The data were analyzed using a digital autocorrelator with a non-negative least-squares (NNLS) method. A distribution of hydrodynamic diameters was obtained based on the Stokes-Einstein equation for the diffusion coefficient of a sphere. All distributions were weighted by volume. Reported average diameters correspond to the D₅₀, or diameter at which the cumulative sample volume was under 50%. For zeta potential measurements, the average value of at least three data points was reported. Thermogravimetric analysis (TGA) was used to determine the amount of adsorbed ligand mass on the primary gold nanoparticles and the final polymer/gold ratio of the nanoclusters. TGA was performed using a Perkin-Elmer TGA 7 under nitrogen atmosphere at a gas flow rate of 20 mL/min. Excess, unadsorbed organic material, either ligands and/or polymer, was removed from particles, either colloidal gold or nanoclusters, by centrifuging the dispersions at 10,000 rpm for 5 minutes at 4° C. For the colloidal gold particles, which were too small to settle efficiently during centrifugation, centrifugal filter devices were used to separate and filter the particles from the smaller unadsorbed ligands. The supernatants were discarded and the pellets were dried to a powder. The powder samples were held at 50° C. for 120 minutes to remove any moisture in the sample and then heated at a constant rate of 20° C./min from 50° C. to 800° C. and held at 800° C. for 30 minutes. The loss in mass after heating accounted for the organic component of the particles. Flame atomic absorption spectroscopy (FAAS) was used to determine the gold concentration in the dispersion and the yield for the gold particles that were incorporated into the clusters. A GBC 908AA flame atomic absorption spectrometer (GBC Scientific Equipment Pty Ltd) was used to determine the amount of gold present in a sample. All measurements were conducted at 242.8 nm using an air-acetylene flame. To determine clustering efficiency, a dispersion of nanoclusters of known concentration was centrifuged at 10,000 rpm for 10 minutes at 4° C. FAAS measurements were conducted on the supernatant.

The stability of the nanoparticles may be quantified using a stability ratio, W, defined as the ratio of the rate of fast, diffusion controlled aggregation to slow, kinetically-controlled aggregation. Alternately, W may also be determined using the respective half-lives for fast and slow aggregation.

$$W = \frac{{k}_{f}}{{k}_{s}} = \frac{{t}_{1 / 2 , s}}{{t}_{1 / 2 , f}} \qquad ( 1 )$$

where k_{f} and kₛ are the rate constants for fast and slow flocculation, respectively, and t_{1/2,f} and t_{1/2,s} are the half-lives for fast and slow flocculation, respectively. The half life for fast, diffusion-controlled aggregation according to Smoluchowski is given as:

$${t}_{1 / 2 , f} = \frac{3 η}{4 {k}_{b} {TN}_{0}} \qquad ( 2 )$$

where η is the solution viscosity, and N₀ is the initial number density of nanoparticles. Slow flocculation half-lives were estimated experimentally based on the observed time required for a visual color change in the nanocluster dispersion to occur, t_{col}. The observed t_{col} may be used to estimate half-lives using the assumption that a color change corresponds to the collision of 11 particles and solving the equation for second order reaction kinetics, 1/N(t)=kt+1/N₀ᵢ, to yield:

t_{1/2,s}=t_{col}/10 (3)

where N is the number of particles in the system at time, t, and k is the reaction rate constant. Effect of particle volume fraction on nanocluster size and optical properties. The amount of ligands on the surface of the gold particles was determined prior to the formation of nanoclusters. For the red citrate-capped gold nanoparticles, the average diameter was 3.8±1.0 nm (data not shown) and the zeta potential was −44.0±4.7 mV (Table 2) at a pH of ˜7.2. Table 2: Zeta potentials of gold primary particles and nanoclusters capped with citrate or a combination of citrate and lysine ligands.

TABLE 2

| Ligand | Zeta potential (mV) |
|---|---|
| Citrate | -44.0 ± 4.9 |
| (primary particle) | |
| Citrate/lysine | -30.1 ± 2.4 |
| (primary particle) | |
| PLA(2K)-b-PEG(10K)-b-PLA(2K) | -8.0 ± 0.2 |
| Citrate/lysine | -16.3 ± 4.0 |
| 16/1 PLA-b-PEG-b-PLA /Au | |
| (nanocluster − 100% evaporation) | |
| Citrate | -13.0 ± 3.3 |
| 16/1 PLA-b-PEG-b-PLA /Au | |
| (nanocluster − 100% evaporation) | |

FIGS. 34A-34F are TEM images of nanoclusters produced after (FIG. 34A) 0%, (FIG. 34B) 50%, (FIG. 34C) 60%, (FIG. 34D) 80%, (FIG. 34E) 100% solvent evaporation. (FIG. 34F) SEM image of nanoclusters produced after 100% solvent evaporation. The nanoclusters were formed at an initial gold concentration of 3 mg/mL and a PLA-b-PEG-b-PLA concentration of 50 mg/mL.

The citrate coverage on the gold nanoparticles was estimated to be about 6.3% w/w, based on calculations assuming saturated ligand coverage on the 4 nm gold particle surface in good agreement with the 7% w/w citrate measured by TGA. The adsorption of lysine to gold did not significantly change the particle size, which was 4.1±0.8 nm (FIG. 34A), nor the pH of the gold dispersion. Lysine contains two NH₃⁺ charges and one COO⁻ charge over a pH range from 3 to 10. FIG. 35 is a schematic of lysine ligand. The ligand exchange with the positively charged lysine increased the zeta potential to −30.1±2.4 mV (Table 2), indicating about 30% of the adsorbed citrate was exchanged. The citrate/lysine-capped particles were coated with 11% total ligand, according to TGA results, compared with 7% for the citrate-only stabilized nanoparticles. The color did not vary for the citrate-only and citrate/lysine-capped gold nanoparticle dispersions for ˜1 month, corresponding to a very high W of ˜7×10⁹ (Table 3) for an N₀ of ˜10²¹ particles/m³ and t_{1/2,f} of 3.93×10⁻⁵ s, which is based on a gold loading of 3 mg/mL.

TABLE 3

| Evaporation | | | | | |
|---|---|---|---|---|---|
| Extent (%) | N_{0 (}particles/m³) | t_{1/2,f} (s) | t_{col} (s) | t_{1/2,s} (s) | W |
| 0, no polymer | 5 × 10²¹ | 3.93 × 10⁻⁵ | 2.59 × 10⁶ | 256634 | 6.60 × 10⁹ |
| 0, polymer | 5 × 10²¹ | 1.51 × 10⁻⁴ | 3.60 × 10³ | 356 | 3.97 × 10⁵ |
| 50 | 1 × 10²² | 1.30 × 10⁻⁴ | 300 | 30 | 2.50 × 10⁵ |

Calculated stability ratios for nanoclusters produced using citrate/lysine-capped nanoparticles at a 16/1 PLA-b-PEG-b-PLA/Au ratio and a starting gold concentration of 3 mg/mL. The high stability is due to strong repulsive charges on the ligands of the particles, in good agreement with previous reports in literature.

To form gold clusters, interactions between citrate/lysine gold particles were mediated with a weakly adsorbing polymer, either PLA-b-PEG-b-PLA, as shown in FIG. 1, or PEG (MW=3350) homopolymer. Without any solvent evaporation, the addition of either polymer to the ligand-capped gold particles at a 16/1 polymer/gold ratio (gold concentration=3 mg/mL) did not produce a color change over a period of one hour, indicating that clusters of closely-spaced gold were not formed. After an hour, the color slowly changed. A one hour stability (t_{col}=1 hr) corresponds to a maximum W of ˜4×10⁵, as determined from Eqs. 1-3 (Table 3). To more fully probe the kinetics of nanocluster formation, the nanocluster size was monitored as a function of solvent evaporation by quenching cluster growth with the addition of DI water after a specified level of solvent evaporation. The harvested nanoclusters were observed by TEM (FIG. 2) and their sizes determined by DLS.

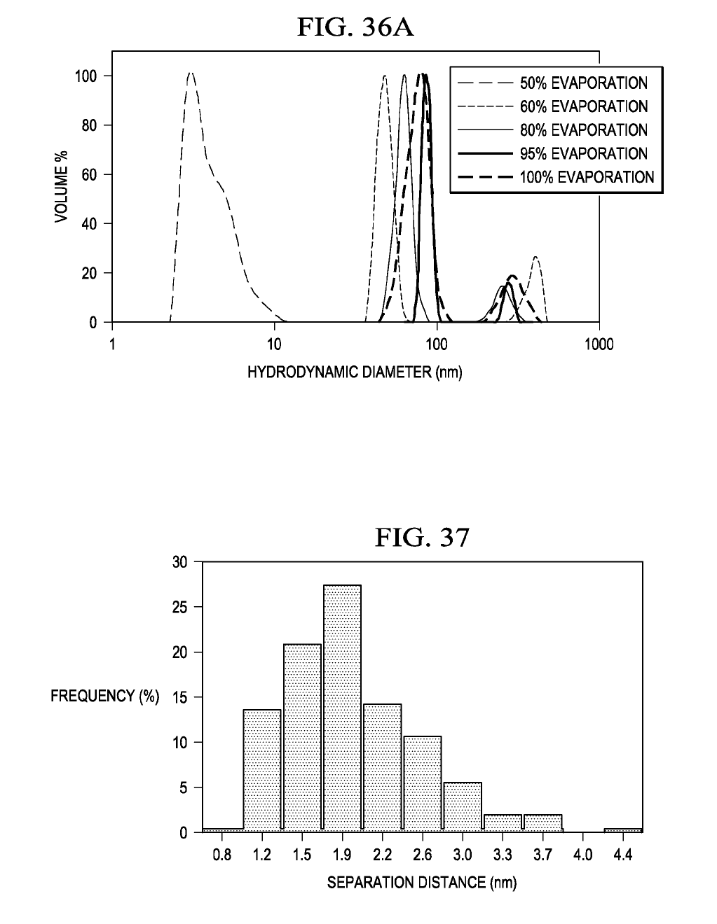

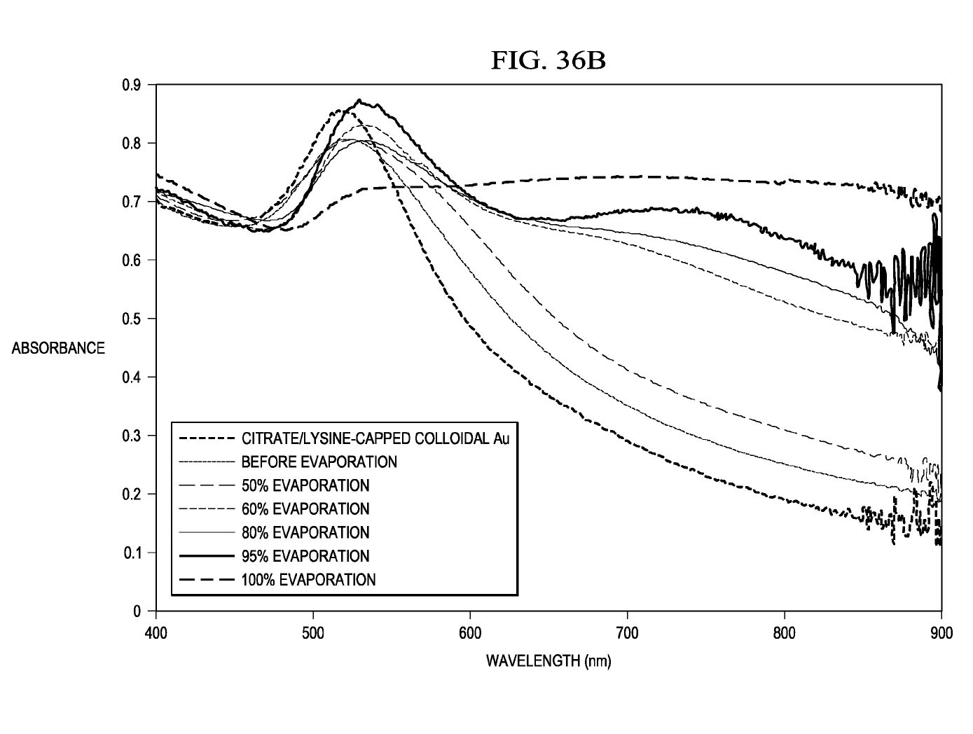

FIG. 36A is an image of the particle size measurements, by DLS, and FIG. 36B is an image of the UV-vis absorbance spectra for nanoclusters composed of citrate/lysine-capped gold nanoparticles produced after different extents of evaporation. Nanoclusters were produced at a starting gold concentration of 3 mg/mL and bound together with PLA-b-PEG-b-PLA at a polymer/gold ratio of 16/1. For PLA-b-PEG-b-PLA, the formation of dimers and trimers was detected, indicating nucleation, after 50% solvent evaporation, which occurred over ˜5 minutes for a 1.4 mL sample. This time corresponds to a maximum W of ˜2.5×10⁵, as t_{1/2,s} could have been even smaller than 5 minutes. These small oligomers produced a shoulder in the DLS size distribution. When the suspension, from which 50% of the solvent had been evaporated, was allowed to sit over the course of one week, still no color change was observed, indicating that the oligomers did not grow to produce larger clusters. However, additional solvent reduction to 60% evaporation, approximately one minute later, led to clusters 35-60 nm in size, as seen both by TEM and DLS. Further solvent evaporation, to 80% and 100%, produced additional growth, with D₅₀ values of 60 nm and 80 nm, respectively, with low polydispersities between 1.1-1.8. From 50 to 95% evaporation, the cluster size was monotonic with the extent of evaporation. Yields of gold in the clusters, or the percent of the loaded primary particles that are incorporated into clusters after quenching the growth, was determined using FAAS (Table 4).

TABLE 4
Size distribution moments and cluster yields, as determined by FAAS, for nanoclusters
produced using different extents of evaporation. The initial gold concentration was 3 mg/mL and
the PLA-b-PEG-b-PLA /gold ratio was 16/1.

| Sample | Cluster yield (%) | μ₁ | μ₃ |
|---|---|---|---|
| Citrate/lysine-capped nanoclusters 100% | 99.7 | 1.11 | 0.93 |
| evaporation | | | |
| Citrate/lysine-capped nanoclusters 80% | 96.8 | 1.04 | 0.97 |
| evaporation | | | |
| Citrate/lysine-capped nanoclusters 60% | 95.1 | 1.09 | 0.93 |
| evaporation | | | |
| Citrate-capped nanoclusters | 98.5 | 1.01 | 0.99 |
| 100% evaporation | | | |

After 60% and 100% solvent evaporation, 95.1% and 99.7%, respectively, of the initially loaded gold nanoparticles by mass were incorporated into clusters. Therefore, cluster yields, as well as size, continued to increase with the extent of solvent evaporation. The ability to tune the cluster size over a wide range and to achieve low polydispersities is of great scientific and practical interest.

Extents of solvent evaporation greater than 60% resulted in a color change of the dispersion to blue, but it was difficult to observe the kinetics given the dark, opaque dispersions at the high volume fractions. Thus, the spectra were measured after the clusters were quenched by dilution. The red shifts in the absorbance to the NIR were consistent with the morphologies observed by TEM and the sizes measured by DLS. Before polymer was added, the characteristic spectrum for individual gold particles exhibited a maximum at 530 nm (FIG. 36B). For the dimers and trimers (FIG. 34B), the red-shift was modest as expected. Much larger shifts were observed for 60% evaporation (FIG. 36B), where sizable clusters of 35-60 nm were observed by TEM and DLS, as expected from theoretical calculations. The NIR absorbance continued to increase as the extent of evaporation and nanocluster size increased.

Complete solvent evaporation produced a smooth blue film on the glass surfaces of the vials, indicating a shift in the absorbance spectra of gold to the NIR. Reconstitution of the film with DI water yielded a dark blue dispersion of sub-100 nm clusters composed of primary gold nanoparticles (FIG. 34). SEM images of nanoclusters formed after 100% solvent evaporation reveal a polymer-rich shell a few nanometers thick surrounding the exterior of each cluster. The spectra of the nanoclusters formed after 100% solvent evaporation exhibited a broad, relatively constant, absorbance in the important NIR region from 700 to 900 nm, corresponding to an extinction coefficient at the maximum absorbance, ∈703, of 0.017 cm2/μg for a 56 μg/mL gold dispersion. Assuming that the gold nanoparticles occupy ˜72% of the cluster volume (based on SEM and TEM images in FIG. 34), characteristic of a closest-packed volume fraction, the estimated particle extinction cross section was 9.0×10-15 m2, comparable to the value for nanoshells, nanocages, nanorods, and nanoroses. The mean spacing between primary gold particles within the clusters was estimated to be 1.80±0.6 nm based on the more discernible particles in the periphery of TEM images, well within the range of interparticle spacing known to produce a significant red-shift in the SPR.

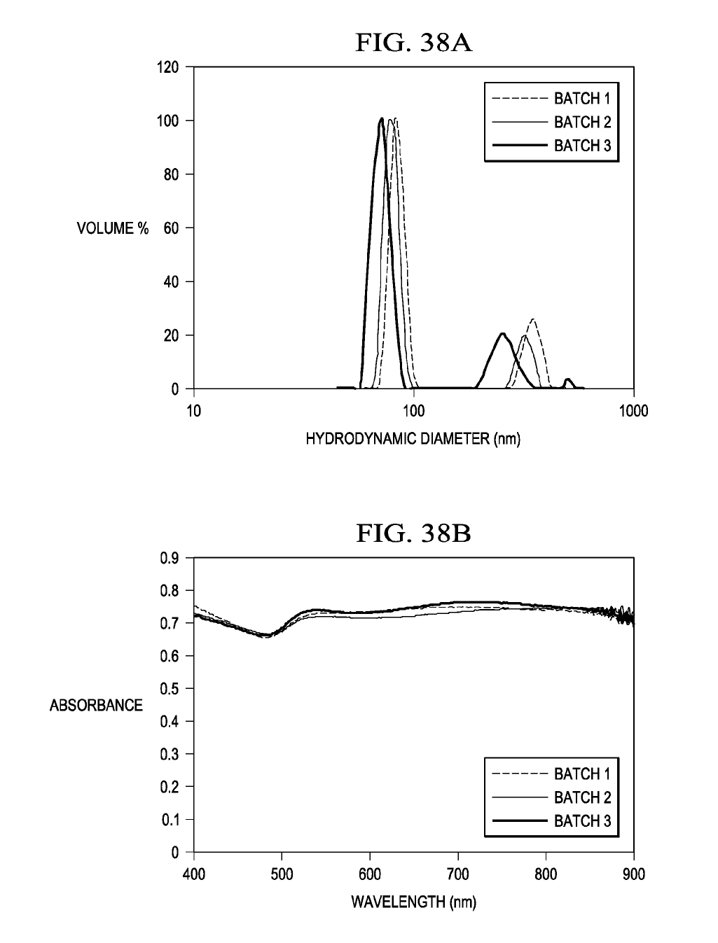

FIG. 37 is a histogram of separation distances between primary gold nanoparticles within a nanocluster produced after 100% solvent evaporation (starting gold concentration of 3 mg/mL and a PLA-b-PEG-b-PLA/Au ratio of 16/1). Measurements taken using particles on the periphery of the nanoclusters. Over 130 measurements were taken. Inset is a TEM image of one of the clusters that was used in this measurement. The ability of the gold nanoparticles to pack tightly together is supported by TGA results, which indicated that after 100% solvent evaporation, nanoclusters contained only 20±5% organic material. From the known amount of ligand reported above, 10-15% of this material was polymer. The ability to reproducibly produce nanoclusters using 100% evaporation, with respect to both size and optical properties, is shown in FIG. 38.

FIG. 38 is an image of the reproducibility of nanoclusters of citrate/lysine-capped gold nanoparticles in terms of (a) size and (b) optical properties. Starting gold and PLA-b-PEG-b-PLA concentrations were 3 and 50 mg/mL, respectively. Nanoclusters were produced after 100% solvent evaporation.

The zeta potentials of the resultant nanoclusters of citrate-only and citrate/lysine-capped nanoparticles were −13.0±3.3 mV and −16.3±4.0 mV, respectively, approximately half that of the initial colloidal gold nanoparticles (Table 2). Interestingly, the zeta potential of clusters formed using citrate-only and citrate/lysine-capped gold, stabilized with PLA-b-PEG-b-PLA, had similar zeta potential values, somewhat larger than that of the pure polymer. The value of −8.0±0.2 mV for the PLA-b-PEG-b-PLA polymer is attributed to the ionized PLA end groups.

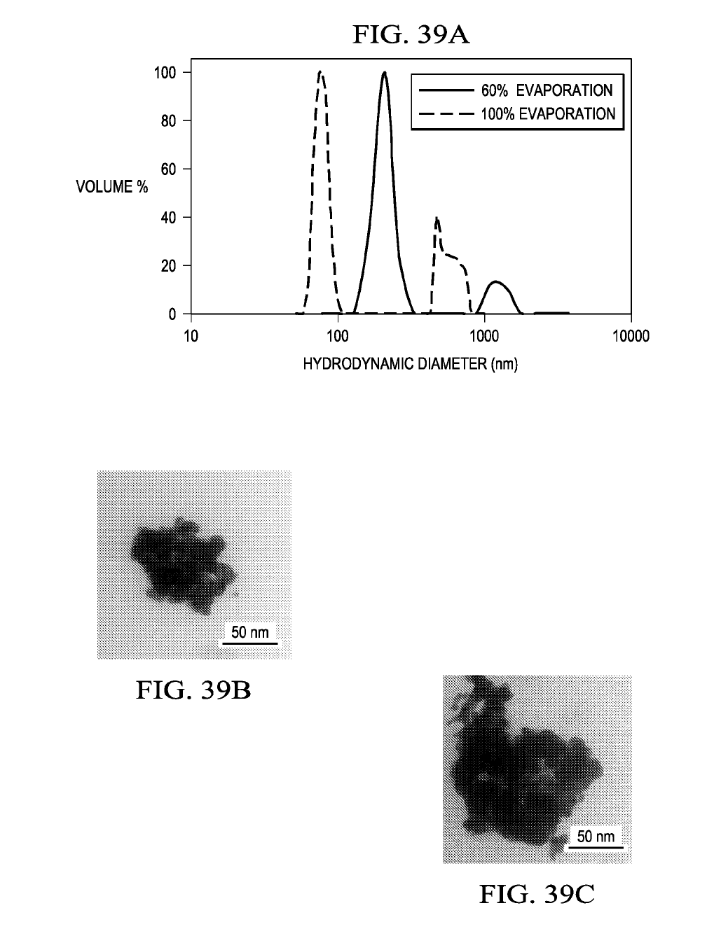

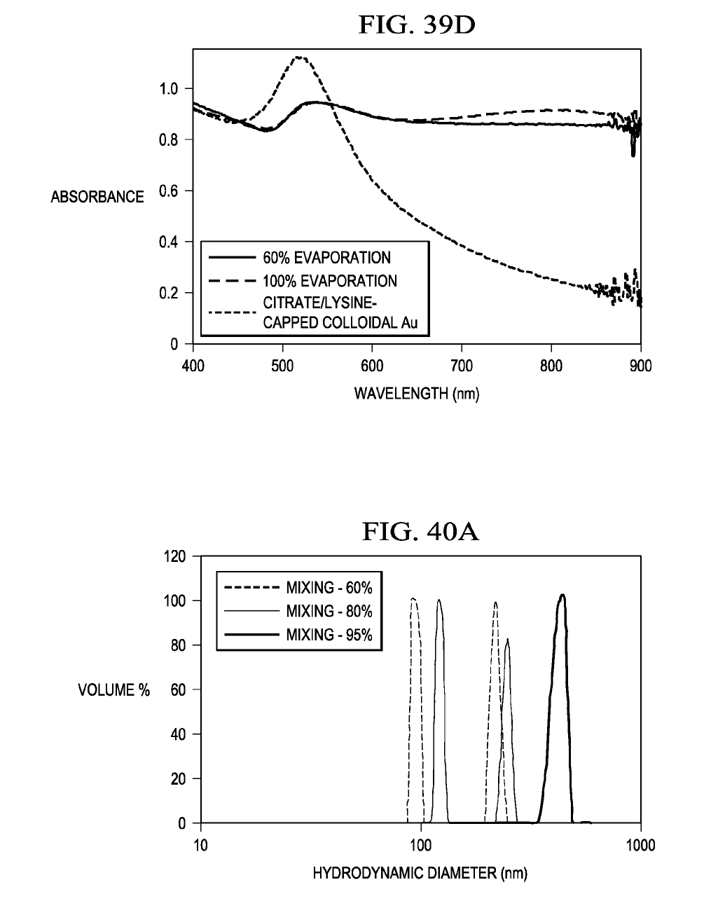

FIG. 39A is an image of the particle size measurements, by DLS, TEM images of nanoclusters after (FIG. 39B) 60% and (FIG. 39C) 100% solvent evaporation, and (FIG. 39D) UV-vis absorbance spectra of nanoclusters composed of citrate/lysine-capped nanoparticles assembled using PEG homopolymer (MW=3350). The starting gold and polymer concentrations were 3 mg/mL and 50 mg/mL, respectively.

Nanoclusters were also formed using PEG (MW=3350), instead of PLA-b-PEG-b-PLA, as the stabilizing polymer. The PEG-stabilized clusters were, on average, ˜1.5 times larger than those stabilized using PLA-b-PEG-b-PLA, as reported by DLS and TEM (FIG. 39A-39C). Similar to observations for PLA-b-PEG-b-PLA-stabilized clusters, a reduction in solvent evaporation from 100% to 60% yielded a ˜30% reduction in cluster size and slightly lower NIR absorbances. The strong NIR absorbance of the PEG-stabilized clusters indicated that tight packing of gold nanoparticles within the cluster was achieved (FIG. 39D). In fact, the clusters formed at 60% solvent evaporation show a slightly stronger NIR peak than the clusters formed after 60% solvent evaporation using PLA-b-PEG-b-PLA, likely due to the larger cluster size. Similar trends were obtained for nanoclusters produced using citrate-only capped gold nanoparticles and PEG 3350.

Assembly of nanoclusters was also demonstrated without solvent evaporation by mixing together highly concentrated gold and polymer solutions. The resulting concentrations of gold particles and polymer corresponded to those achieved by certain solvent evaporation extents. For example, a 6 mg/mL dispersion of gold nanoparticles was mixed with a 100 mg/mL polymer solution to produce clusters that were equivalent to the concentrations achieved after 50% evaporation. However, the cluster sizes were at least 2.5 times larger than those where the particle volume fractions were increased gradually by solvent evaporation.

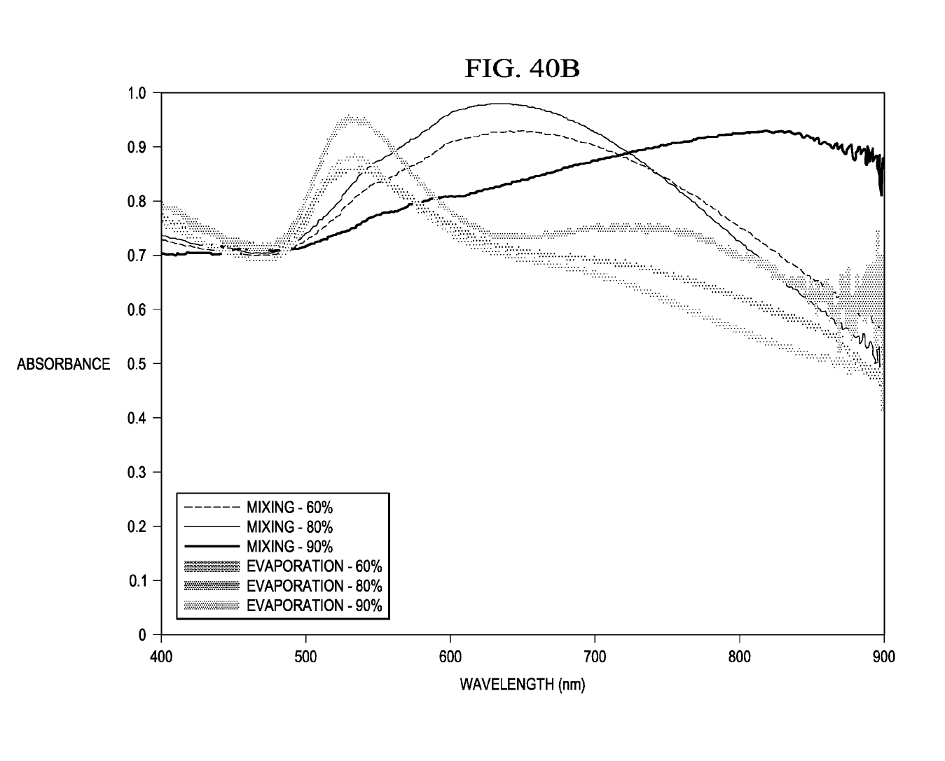

FIG. 40A is an image of the particle size distribution, as measured by DLS, and FIG. 40B is an image of the UV-vis spectra of clusters of citrate/lysine-capped nanoparticles made with the mixing protocol. The conditions of cluster formation are equivalent to that for clusters formed by solvent evaporation at a starting gold concentration of 3 mg/mL and a PLA-b-PEG-b-PLA/Au ratio of 16/1. In FIG. 40B is an image of the UV-vis spectra are compared to that for nanoclusters produced using solvent evaporation. Because of their larger sizes, nanoclusters produced by this method displayed even more shifted NIR absorbance (FIG. 40B). Similar trends in optical properties were observed when clusters of citrate-only-capped gold nanoparticles were produced using this mixing method.

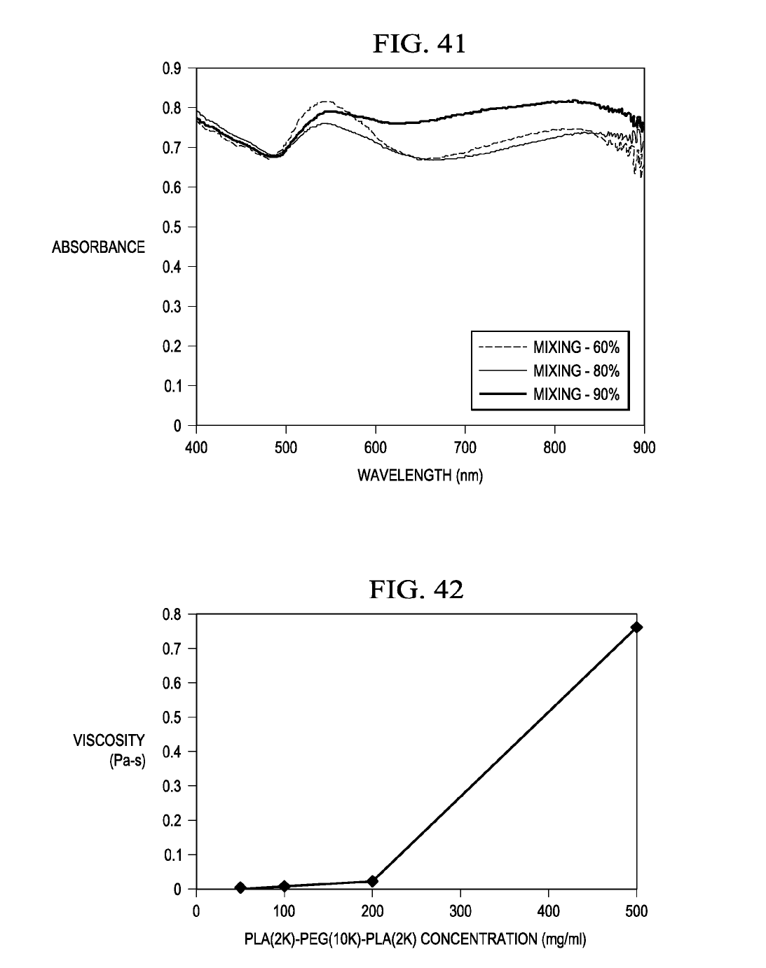

FIG. 41 is an image of the UV-vis spectra of clusters of citrate-capped nanoparticles made with the mixing protocol. The starting gold concentration was 3 mg/mL and the PLA-b-PEG-b-PLA/Au ratio was 16/1.

The high viscosities of the extremely concentrated polymer solutions, ranging from 9×10-4 Pa s (˜10 times that of water) to 0.8 Pa s (˜900 times that of water) for solutions corresponding to 60% and 90% solvent evaporation, respectively, resulted in inadequate mixing rates, poorer polymer diffusion, and thus the larger clusters.

FIG. 42 is an image of the viscosity of PLA-b-PEG-b-PLA as a function of concentration. Viscosity measurements were performed using a cone and plate viscometer (TA Instruments AR 2000ex with a Peltier plate base and aluminum cone, with a diameter of 40 mm, angle of 1o 59 minutes and 56 seconds and a truncation distance of 55 μm).

Nanoclusters were produced using gold nanoparticles capped with two other types of ligands: negatively charged citrate, and neutral PEG-SH to compliment the above studies which used lysine (positively charged) and citrate ligands, simultaneously. Clusters of gold primary particles capped with either citrate or a citrate/lysine mixture exhibited strong NIR absorbance.

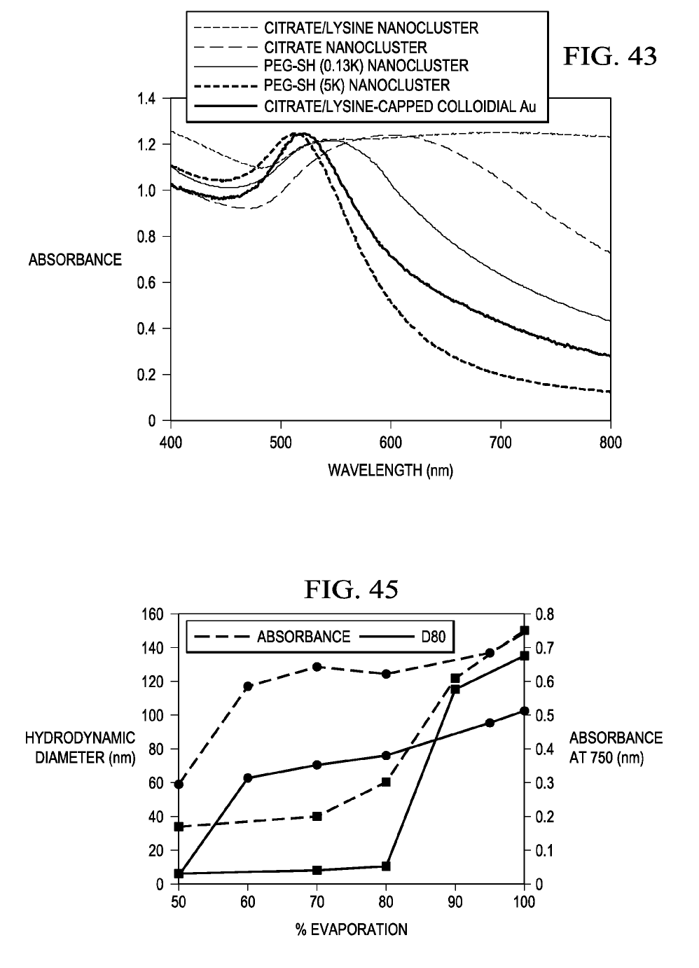

FIG. 43 is an image of the UV-vis absorbance spectra for clusters made with gold primary particles capped with different ligands. The clusters were produced using a starting gold concentration of 3 mg/mL and bound together using PLA-b-PEG-b-PLA at a 16/1 polymer/Au ratio. The clusters were formed under 100% solvent evaporation. However, nanoparticles capped with PEG-SH did not produce a significant red-shift, although the shift was larger for PEG-SH with a MW of 0.13K versus 5K. PEG-SH 5K has a reported radius of gyration of 3.1 nm. Therefore, the corresponding particle separations between two PEG-SH coated particles of at least 6.2 nm is larger than the diameter of a gold primary particle and the strongly bound PEG-SH 5K ligands prevented the gold nanoparticles from packing together tightly enough for a strong red shift. Relative to citrate/lysine-capped particles, very similar behavior was observed for clusters assembled with citrate-only capped gold nanoparticles and PLA-b-PEG-b-PLA upon solvent evaporation, according to DLS, TEM, and UV-vis/NIR measurements.

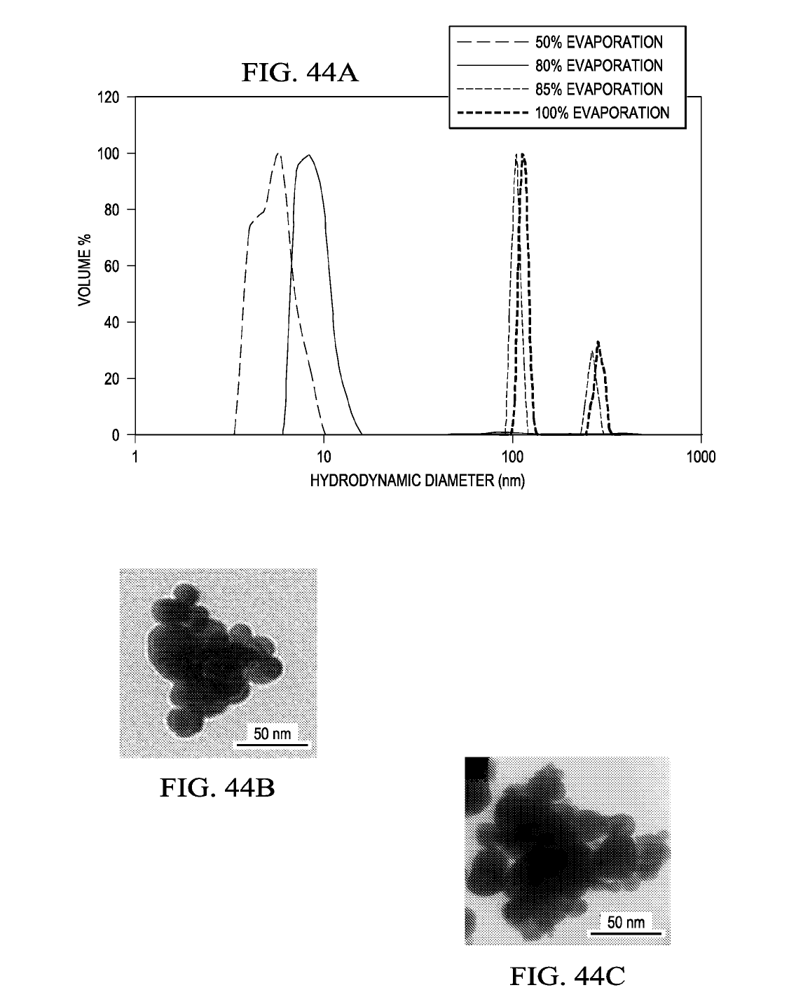

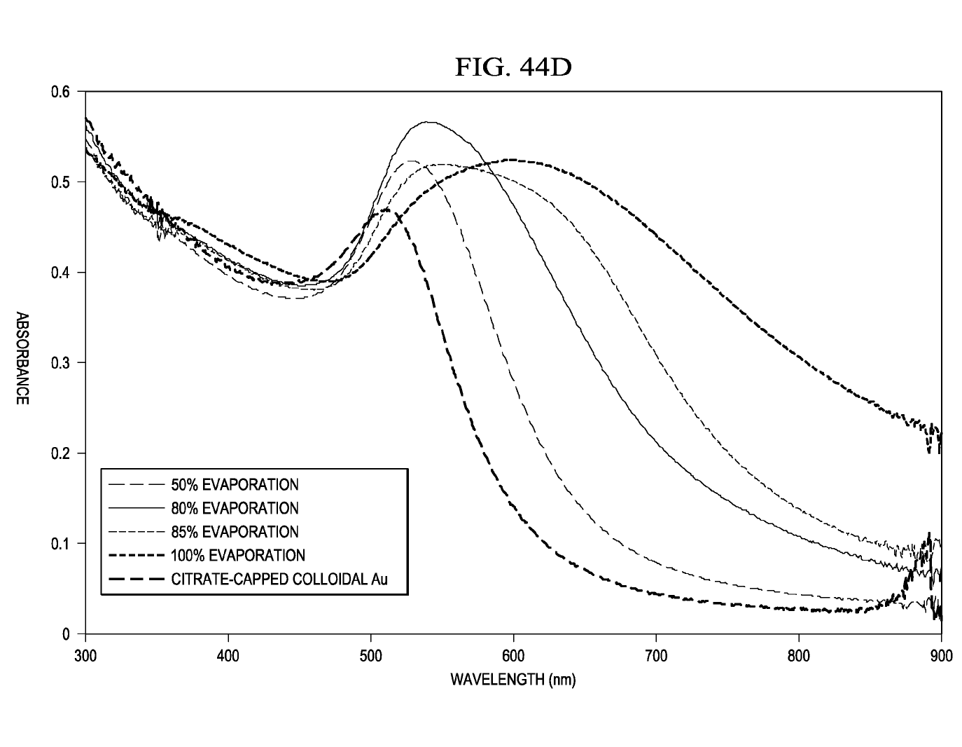

FIG. 44A is an image of the DLS measurements, TEM images after (FIG. 44B) 85% and (FIG. 44C) 100% solvent evaporation, respectively, and (FIG. 44D) UV-vis, absorbance spectra for nanoclusters composed of citrate-capped gold nanoparticles produced after different extents of evaporation with a starting gold concentration of 3 mg/mL and a PLA-b-PEG-b-PLA/gold ratio of 16/1.

Again, there was a very strong correlation between cluster size and NIR absorbance. However, the clusters did not form until ˜85% solvent evaporation, as compared to 60% for citrate/lysine capped gold.

FIG. 45 is an image of the Hydrodynamic diameter (D80) and absorbance values for nanoclusters composed of primary particles capped with citrate (▪) or a combination of citrate and lysine () ligands. The clusters were produced using a starting gold concentration of 3 mg/mL and bound together using PLA-b-PEG-b-PLA at a 16/1 polymer/Au ratio. The greater repulsion for the citrate-only-capped particles, as is evident in the larger zeta potentials, appeared to delay cluster formation. The slightly smaller sizes and larger SPR red-shifts of the nanocluster composed of citrate/lysine nanoparticles may be influenced by the attractive electrostatic attraction between the positive and negative charges on the lysine. These interactions may further promote polymer exclusion from the cluster interior.

To demonstrate the ability to tune the cluster size, the gold loading was lowered to 1.0 mg/mL, compared to 3.0 mg/mL in our previous study, and the polymer/gold ratio was varied over a wide range for 100% solvent evaporation.

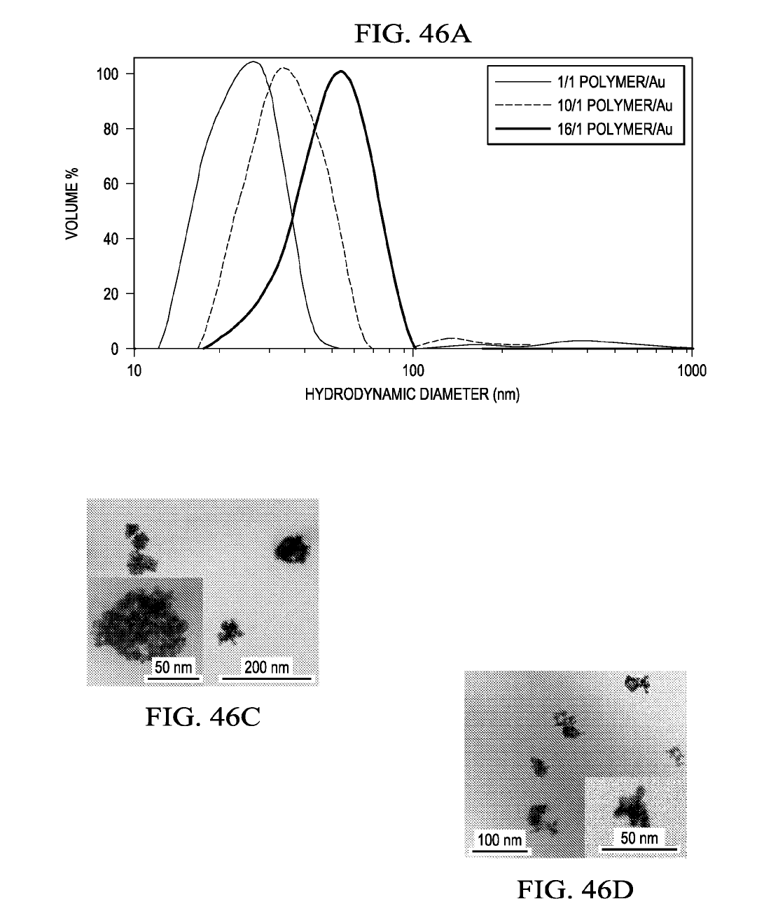

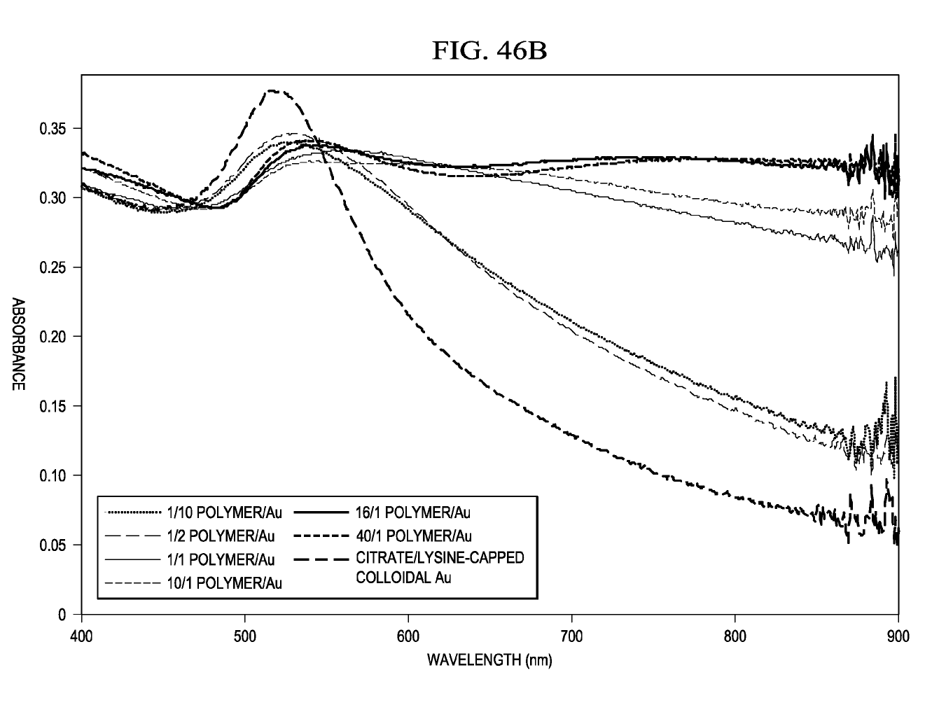

FIG. 46A is an image of the particle size distribution, as measured by DLS, and FIG. 46B is an image of the UV-vis absorbance spectra of nanoclusters of citrate/lysine-capped nanoparticles produced with varying PLA-b-PEG-b-PLA/gold ratios at an initial gold concentration of 1 mg/mL and 100% solvent evaporation. TEM images of nanoclusters: (FIG. 46C) 16/1 polymer/gold ratio and an initial gold concentration of 3 mg/mL and (FIG. 46D) a 1/1 polymer/gold ratio with an initial gold concentration of 1 mg/mL after 100% solvent evaporation.

Cluster sizes decreased considerably as polymer/gold ratios were reduced from 16/1 to 1/1 (FIG. 46A), with an average diameter of 28.4 nm for the 1/1 ratio. Despite the reduction in cluster size, clusters produced at a polymer/gold ratio between 1/1 to 16/1 still exhibited a broad and intense NIR absorbance, similar to that shown in FIG. 36B. However, for polymer/gold ratios below 1/1, the absorbance did not shift significantly from that of colloidal gold (FIG. 46B).

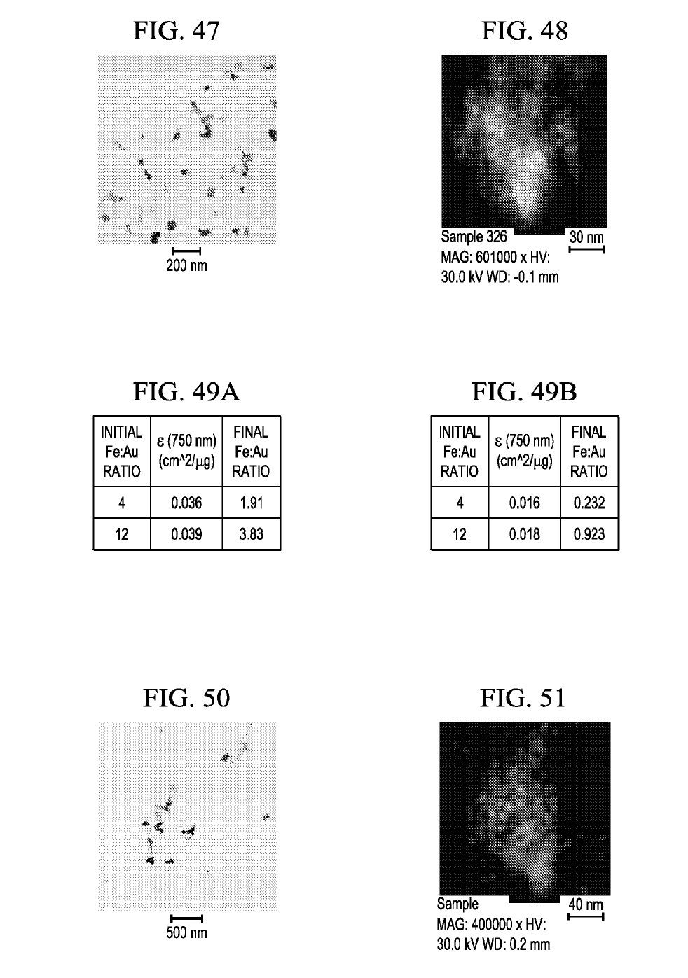

FIG. 47A is an image of the particle size measurements by DLS and FIG. 47B is an image of the UV-vis absorbance spectra of clusters of citrate/lysine-capped nanoparticles formed when varying the PLA-b-PEG-b-PLA/Au ratio. The starting gold concentration was 3 mg/mL and the clusters were formed under 100% solvent evaporation. For a given polymer/gold ratio, similar results were obtained for the cluster size and spectra for the higher gold loading of 3.0 mg/mL, as shown in FIG. 47A, although the sizes were slightly smaller for the 1.0 versus the 3.0 mg/mL loading. As an example of the extent by which the cluster sizes could be tuned, the much smaller clusters formed with a 1/1 polymer/gold ratio at a gold loading of 1.0 mg/mL versus a 16/1 polymer/gold ratio at a 3.0 mg/mL loading is shown in TEM micrographs (FIGS. 46C-46D). Additionally, a small decrease in the absorbance spectra was observed for clusters formed at a 40/1 polymer/gold ratio and a 3.0 mg/mL gold loading (FIG. 47B). Here, an extremely high polymer concentration of 1200 mg/mL was generated when the level of solvent evaporation reached 90%, resulting in excessive polymer that likely interfered with close-spacing between the gold nanoparticles, and thus, lowered the red shift. This interference was not present for lower polymer/gold ratios. Further decreasing gold loadings as low as 0.19 mg/mL and increasing the polymer/gold ratio up to 260/1 led to the formation of increasingly larger clusters with reduced NIR absorbance.

Particle sizes, as determined by DLS, of citrate/lysine-capped nanoclusters formed when varying the starting concentration of the colloidal gold solution. The starting PLA-b-PEG-b-PLA concentration was 50 mg/mL.

| Sample | Particle Size Range (nm) |
|---|---|
| C₀ = 0.19 mg/mL Au | 74-118 (12%), 380-608 (80%) |
| C₀ = 0.38 mg/mL Au | 82-122 (62%), 502-613 (38%) |
| C₀ = 0.75 mg/mL Au | 44-57 (68%), 250-377 (32%) |
| C₀ = 1.5 mg/mL Au | 56-100 (89%), 316-562 (11%) |
| C₀ = 3.0 mg/mL Au | 54-101 (82%), 236-359 (18%) |

FIG. 48 is an image of the UV-vis absorbance spectra of citrate/lysine-capped nanoclusters formed when varying the starting concentration of the colloidal gold solution. The starting PLA-b-PEG-b-PLA concentration was 50 mg/mL.

Nanoclusters produced at a 1/1 gold/polymer ratio and a 1.0 mg/mL gold concentration were approximately 85% gold w/w, comparable to 80% w/w gold in nanoclusters formed with a 16/1 gold/polymer ratio and a starting gold concentration of 3.0 mg/mL, as determined by TGA.

The kinetic assembly of nanoparticles into clusters may be controlled by adjusting the stability ratio for a pair of particles, which is dependent upon the total interaction potential between particles:

Vₜₒₜₐₗ=V_{electrostatic}+V_{VDW}+V_{steric}+V_{depletion} (4)

The first two terms are described by DLVO theory. The addition of a weakly or non-adsorbing polymer introduces attractive depletion interactions, which arise from the exclusion of polymer from the gap region between two particle surfaces. The depletion potential for hard sphere colloids and polymers treated as “penetrable hard spheres” is given by:

$$\frac{{V}_{depletion} ( H )}{{k}_{b} T} = - {ρ}_{∞} π [ \frac{4}{3} {r}^{3} + 2 {r}^{2} a - {r}^{2} H - 2 raH + \frac{{aH}^{2}}{2} + \frac{{H}^{3}}{12} ] , \\ 0 \leq H < 2 r \qquad ( 5 )$$

where H is the distance between particle surfaces, r is the polymer radius, a is the nanoparticle radius, and ρ∞ is the number density of polymer particles in solution. If the polymer forms micelles, the micellar properties are used. The ability of depletion forces to cause particle flocculation, and even phase separation, in colloid-polymer mixtures is well known both experimentally and theoretically. The kinetic stability ratio, in terms of Vₜₒₜₐₗ, is described by

$$W = 2 a {\int }_{2 a}^{∞} \frac{\frac{{D}_{∞}}{D ( u )} [ exp ( \frac{{V}_{total}}{{k}_{b} T} ) ]}{{H}^{2}} d H \qquad ( 6 )$$

where u is a dimensionless variable defined as (H−2a)/a, and the ratio D∞/D(u) is the hydrodynamic correction factor:

$$\frac{{D}_{∞}}{D ( u )} = \frac{6 {u}^{2} + 13 u + 2}{6 {u}^{2} + 4 u} \qquad ( 7 )$$

The first parts of the discussion section compare the kinetically controlled nanocluster assembly with previous studies based on the terms for Vₜₒₜₐₗ and the manipulation of the particle concentrations. A quantitative expression is not presented herein for V_{steric}, given the complexity of hydration of PEG at high concentrations where gels are formed.

In the absence of a polymer, the VDW and electrostatic terms play a primary role in cluster formation, whereas steric and depletion interactions are small. Electrostatic repulsion of the nanoparticles may be weakened by a change in pH or salinity to reduce the charge. For dilute dispersions of gold coated with citrate (0.1 mg gold/mL), the growth from attractive VDW forces may be controlled over a period of hours to form clusters >100 nm in size. For these dilute conditions, the clusters are typically relatively low density with a low fractal dimension. In contrast, clusters formed at high particle concentrations are more likely to be composed of gold particles with close spacing that favors strong NIR absorbance. However, for concentrated gold dispersions (20-50 mg/mL), it becomes difficult to balance the electrostatic repulsion and VDW attraction to control the growth, and substantial aggregation has been observed over a period of several minutes. For instance, when gold nanoparticles are capped with lysine ligands, a change in pH simultaneously produces both positive and negative charges (FIG. 35) that result in electrostatic attraction and irregularly shaped aggregates up to several microns in diameter. Additional concepts in kinetic assembly are needed to better control Vₜₒₜₐₗ and thus the particle size and gold spacing

The key challenge in this study was to control nanocluster size and gold particle spacing within the clusters by manipulation of the particle concentration pathways and Vₜₒₜₐₗ. High gold particle concentrations (>>0.1 mg/mL) were utilized in order to achieve sufficiently close gold particle spacing for strong NIR absorbance. However, they can also cause unmitigated cluster growth. This dilemma was addressed by the addition of a weakly adsorbing polymer to manipulate the electrostatic, steric, and depletion forces. The polymer initiates nucleation and growth, while simultaneously providing steric stabilization, but with low final polymer loadings.

The initial citrate-only and citrate/lysine-capped gold nanoparticles in this study were extremely stable, evidenced by large negative zeta potentials of −44 and −30 mV, respectively, and a Vₜₒₜₐₗ of at least 23 kBT.

FIG. 49 is an image of the Van der Waals and total interaction potentials describing the stability of citrate/lysine-capped gold nanoparticles in the absence of PLA-b-PEG-b-PLA and after the addition of PLA-b-PEG-b-PLA. Effects of solvent evaporation on the total interaction potentials are shown.

Nanocluster formation was initiated by raising the polymer and gold particle concentrations either by solvent evaporation or mixing to raise the adsorption of the polymer on gold. The weakly adsorbed polymer decreases the local dielectric constant near the charged ligands and thus weakens the ion hydration, causing ion pairing. This decrease in particle charge is directly evident in the decrease in the zeta potential with the addition of polymer (Table 2). The decrease in electrostatic repulsion causes a marked decrease in the experimentally determined W (Table 3) from ˜1010 for the citrate/lysine-capped primary particles to ˜105 after the addition of polymer and 50% solvent evaporation. At this condition, the polymer adsorption did not reduce the particle charge enough to produce clusters larger than dimers or trimers within several hours. At an extent of 50% solvent evaporation, the charge on an individual gold particle was regressed from the theoretical W in Eq. 6, given the known experimental W described above (Table 3). In this regression, Vₜₒₜₐₗ included electrostatic, VDW, and depletions terms. All of the properties were known except the surface potential (and thus surface charge) on a gold nanoparticle. The reduction in the regressed surface charge of 1.6 after 50% solvent evaporation, relative to that of the initial colloidal gold particles, was found to be comparable to the reduction in zeta potential given in Table 2. The loss in charge is further characterized by the large decrease in Vₜₒₜₐₗ to about 11 k_{B}T (FIG. 49), which may be attributed to the significant drop in V_{electrostatic} upon charge reduction caused by the polymer, as V_{VDW} did not change. Thus, this large decrease in V_{electrostatic}, and consequently Vₜₒₜₐₗ, produced a decrease in W at 50% solvent evaporation of 5 orders of magnitude, relative to the initial colloidal gold particles.

FIG. 50 is an image of the Stability ratio of a system of citrate/lysine-capped gold nanoparticles in the absence and presence of PLA-b-PEG-b-PLA determined using DLVO theory, as a function of particle volume fraction. It was not possible to regress any changes in the particle charge with higher extents of solvent evaporation because the dispersions were too turbid to determine W experimentally. The regressed charge at 50% was used to calculate the Vtotal and thus W for greater solvent evaporation levels. Vtotal decreased as solvent evaporation increased, primarily due to a reduction in Velectrostatic. Using Eq. 6, the steady decrease in Velectrostatic, and thus Vtotal, with solvent evaporation (i.e. increasing particle volume fraction) was found to cause a further decrease in W (FIG. 50). The Velectrostatic decreases with an increase in the number density of charged gold nanoparticles as the extent of evaporation increases. For electro-neutrality, the resulting increase in counter-ion concentration reduces the Debye length.

FIG. 51 is an image of the DLS measurement of PLA-b-PEG-b-PLA micelles prior to solvent evaporation and after solvent evaporation. A 50 mg/mL polymer solution was prepared. To measure the micelle size, the solution was diluted to 1 mg/mL for analysis by DLS. To determine the effect of solvent evaporation on the polymer, the solution was evaporated to dryness and then redispersed in DI water to a concentration of 5 mg/mL.

However this change in Velectrostatic changes W by less than an order of magnitude, significantly smaller than the changes observed with polymer induced ion pairing. Therefore, the initial cluster growth is driven primarily by the attractive VDW forces upon reduction of particle charge and electrostatic repulsion upon weak polymer adsorption. As the number of closely-spaced gold particles in the cluster increases, the number of water molecules in the coordination shells about each particle decreases, given that the gold surface is hydrophobic. This decrease in hydration may further contribute to ion pairing and weakened electrostatic repulsion.

The smaller clusters produced using PLA-b-PEG-b-PLA as a stabilizer versus PEG homopolymer may be attributed to the stronger adsorption of the more hydrophobic PLA-b-PEG-b-PLA, which produces greater charge reduction and thus more rapid nucleation. The larger number of nuclei and greater steric stabilization for reduced growth would lead to small clusters. Furthermore, the presence of micelles for PLA-b-PEG-b-PLA may provide greater steric stabilization than the homopolymer in the early stages of growth. Similarly, smaller clusters formed for the less charged citrate/lysine-capped gold versus citrate-only capped gold (FIG. 45) may also be attributed to more rapid nucleation. In addition, the attractive electrostatic interactions between the lysine ligands may enhance polymer exclusion from the cluster interior.

The decrease in Velectrostatic to drive cluster growth may also be achieved simply by adding salts. However, without the steric and depletion contributions to the potential, control over the final cluster size for high initial gold particle concentrations has not been successful. Thus, manipulation of these additional terms with polymer concentration and structure is important to achieve greater control over kinetic self-assembly. The nucleation of clusters via an adsorbed polymer to reduce the surface charge and simultaneously provide steric stabilization enables significantly improved control over cluster growth even with the high gold particle concentrations.

The final polymer weight fraction in the clusters was only on the order of 10 to 15% w/w according to TGA, even with starting polymer/gold ratios well above unity, for example our most common case of 16/1. The small spacing between the gold particles of only 1.80 nm (FIG. 37) for PLA-b-PEG-b-PLA stabilized nanoclusters is considerably smaller than the size of a PLA-b-PEG-b-PLA polymer micelle, measured to be 10-14 nm (FIG. 51) or the Rg of the PEG homopolymer of 6.1 nm. Thus, the polymers were excluded from the cluster interior. Various properties of gold contribute to the low polymer loadings, which favor small interparticle distances. The Hamaker constant is 60 kBT for Au versus only 0.6 kBT for the PEG, calculated using Lifshitz theory. The gold surface is not highly hydrophilic given that polypropylene oxide adsorbs more strongly to gold than PEG. Thus, the gold particles are strongly attracted to each other by VDW and hydrophobic forces. Additionally, the polymer chains are depleted from the overlap regions in the interior of the clusters towards the cluster exterior in order to raise their conformation entropy, as described by Eq. 5. These depletion forces, along with the propensity for hydrophilic PEG segments to orient towards the aqueous exterior, drive the weakly adsorbed and hence highly mobile polymer away from the cluster interior and towards the exterior cluster interface with water and into bulk water. This mechanism is supported by the polymer shell observed in the SEM image (FIG. 34F), as well as the low polymer loadings. Thus, the hydrophilic PEG segments of the polymer, which are oriented preferentially towards the exterior cluster interface, extend into the aqueous environment and provide steric stabilization. In essence, the close spacing of the gold particles is driven by the strong VDW attraction between the gold particles and the depletion forces which exclude the polymer.

In the case where a strongly adsorbing polymer is used to regulate cluster formation and growth, the polymer is often retained at significantly higher levels within the final cluster than in the present study. Prud'homme et al. have developed a “flash nanoprecipitation” method to mix an organic dispersion of gold and aquous phase containing a polymeric stabilizer. The process resulted in relatively high 35% w/w particle loadings in clusters by inducing high supersaturation with rapid “micro-mixing” to kinetically control nucleation and growth. The polymer adsorption was sufficiently strong to passivate the surface of nucleating particles under high supersaturation conditions to produce clusters as small as 80 nm. However, the resultant clusters did not exhibit a red-shift into the NIR. It is possible that the interactions between the polymer and the gold were too strong to achieve close-packing between the gold particles. In addition, the organic phase may have attracted too much polymer to the gold.

Size distribution moments calculated from DLS results (FIG. 36) suggest that the nanoclusters were formed more by condensation than by coagulation, yet some coagulation was present. A high yield of 95% of gold in the cluster was observed after only 60% solvent evaporation. Here, exhaustion of primary particles slows down nanocluster growth by condensation. The substantial growth in cluster size from 60% to 100% solvent evaporation cannot be caused by the remaining 5% gold, since the mass of the clusters is proportional to the diameter cubed. Thus, coagulation was the primary cause of growth at this stage. Close inspection of the TEM images in FIG. 34 shows that the larger nanoclusters, formed after larger extents of evaporation (i.e. greater than 60%), are more irregular in shape relative to a spherical geometry. In fact, one may even discern that the larger clusters are partially composed of smaller, 35-60 nm, clusters, indicating a small degree of coagulation. By quenching the nanocluster dispersion with DI water soon after cluster formation, after only 60% solvent evaporation, the potential for additional coagulation was reduced, thus preserving smaller nanocluster sizes and low polydispersities.

A reduction in the polymer/gold ratio from 16/1 to 1/1 resulted in a marked decrease in cluster size from ˜80 nm to ˜30 nm (FIG. 46), as well as a reduction in polymer loading from 20 to 15%, as shown by TGA. This decrease is the opposite of what is expected for steric stabilization alone, indicating other factors were operative. For lower initial polymer/gold ratios and thus polymer concentrations, the lower adsorption onto gold produces a smaller degree of ion pairing and thus a larger Velectrostatic. The greater repulsion will favor slower growth as observed. Furthermore, the lower polymer concentration reduces the collision frequency between polymer chains and gold clusters, leading to less trapping of polymer in the clusters. Rheological factors are also present. The viscosity of PLA-b-PEG-b-PLA solutions increases markedly with concentration in the dilute to semi-dilute transition (FIG. 42). During gold cluster formation via solvent evaporation, the viscosity of the dispersion will increase sooner for higher polymer/gold ratios, increasing the amount of entangled polymer that may get trapped within the gold clusters. This behavior was observed as the polymer/gold ratio was raised from 1/1 to 16/1, and was even more prevalent for the 40/1 polymer/gold ratio (FIG. 47). Coagulation was particularly evident at this highest ratio, according to size distribution moment calculations (μ1=1.55, μ3=0.81). To examine the effect of polymer gelation, a 50 mg/mL solution of PLA-b-PEG-b-PLA without gold particles was dried by solvent evaporation. The precipitate was redispersed to give large aggregates (>500 nm) that did not break up into block copolymer micelles, indicating that gelation was not fully reversible (FIG. 51). For the formation of gold clusters, the gelation of the polymer may make the polymer less available for steric stabilization. Finally, the depletion attraction forces mediate cluster growth both during condensation and coagulation. For smaller polymer/gold ratios, the depletion attraction will decrease, which would favor smaller clusters, as observed (FIG. 46). As the volume of the gap region increases between particles, the depletion attraction also increases. Thus, the depletion attraction will be larger for two 20 nm, growing clusters than for two primary colloidal 5 nm gold particles. Thus depletion attraction may play a larger role in the later coagulation stage than for the initial growth of the smallest embryos.

The mechanism by which our nanoclusters are formed is fundamentally different from equilibrium-based processes, in which particles are assembled into the cores of micelles or at the interface between the core and the corona. In the case of thermodynamic self-assembly, the polymer-gold interactions are inherently stronger and play a much more dominant role, leading to higher polymer loadings and larger gold spacings.

The loadings into micelles are governed by entropic and enthalpic interactions between the solute and the micelle core, as well as the interfacial free energy between the core and corona of a micelle, Δ F̅ᵢ̅ₙ̅ₜ̅. The change in free energy for mixing solute molecules and micelles is given by

Δ F̅₁̅=−ΔSₘ+ΔHₘ+Δ F̅ᵢ̅ₙ̅ₜ̅ (8)

where ΔSm and ΔHm are the change in entropy and enthalpy upon mixing, respectively. The amount of work required for expansion of the interface between the core and corona upon imbibing a solute molecule increases as the micelle size decreases, due to larger Laplace pressures. This interfacial term becomes especially significant for micelles smaller than 200 nm. The loadings of small molecules such as pharmaceuticals in the cores of micelles are often less than 25% by weight and typically less than 10%. The loading of a gold particle in a micelle will be even lower because ΔSm will be less favorable, given the high molecular weight of the particle. For example, loadings of only <2% w/w of ˜2.4 nm gold particles in ˜20 nm polymer micelles has been observed using small angle x-ray scattering (SAXS). Thus, thermodynamic assembly methods are not likely to incorporate sufficient gold loadings to yield a strong red-shift in the SPR for clusters, especially for sizes smaller than 50 nm.

The kinetic nanocluster assembly method in the present study is not restricted by the thermodynamic constraints of micelle encapsulation. Clusters were formed by purposely aggregating gold nanoparticles with a weakly adsorbing polymer to control nucleation and growth by manipulation of the electrostatic, steric, and depletion interactions. The strong van der Waals interactions between the gold particles were the primary driving force for cluster growth. Furthermore, depletion effects promote exclusion of the polymer to the cluster surface. These interactions lead to much higher loadings than for thermodynamic assembly of gold particles with micelles.

Gold nanoparticles with intense NIR absorbance, including nanoshells, nanorods, and nanocages, have received extensive attention as biomedical imaging and therapeutic agents. However, while these particles are within the optimal size range of 6-100 nm to exhibit sufficiently long blood residence times for accumulation at disease sites, they are above the threshold size of 5.5 nm required for efficient clearance by the kidneys. Furthermore, the metallic bonds between the gold atoms in these particles do not biodegrade. In contrast, our gold nanoclusters, using PLA-b-PEG-b-PLA as the stabilizer, were shown to biodegrade nearly completely in solution and in macrophage cells back to the original 5 nm gold spheres. The ability to further tune the size to 30 nm and to vary composition, as demonstrated in the current study, broadens the scope of biodegradable nanoclusters significantly.

Gold nanoparticles (<5-nm) stabilized with citrate or similar ligands were synthesized based on a well known method for reduction of 1% HAuCL₄.3H₂O with 0.075% NaBH₄ in a 1% Na₃C₃H₅O(COO)₃.2H₂O solution. The iron oxide nanoparticles were synthesized by alkaline hydrolysis of iron chlorides. The polymer was designed to influence the polymer interactions with the particle surfaces based on charge and hydrophobic interactions to influence nanocluster nucleation and growth as well as steric stabilization. We formed core-shell clusters with a gold nanocluster core to provide strong NIR absorbance and a shell of iron oxide nanoparticles to give a high magnetization and r2 relaxivity. In this sequential approach, the gold cores were formed first. Gold clusters were formed by mixing a solution of lysine/citrate capped primary gold nanoparticles (approx 3-5 nm in diameter) with various w/w ratios of PEG-b-PPG-b-PEG. The solvent was then evaporated, resulting in an increase in the volume fraction of particles, until a film was formed, and the film was redispersed in a solution of primary iron oxide nanoparticles (approx. 5 nm in diameter). The resulting solution was then concentrated into a film via solvent evaporation and redispersed in an aqueous solution of 1% polyvinyl alcohol, leading to the final dispersion of mixed nanoclusters. The cores acted as seeds to then add the iron oxide particles in the shell. After redispersion, the resulting solution was centrifuged twice at 8000 rpm for 5 min each, in order to separate the small unclustered primary gold and iron oxide particles from the larger nanoclusters. The supernatant containing unclustered gold and iron oxide particles was then separated from the pellet which contained the mixed nanoclusters. The pellet was then redispersed in deionized water and probe sonicated in order to form a stable dispersion. The resulting particles were analyzed to determine optical properties, size, composition, and magnetic properties.

FIG. 47 is a TEM image and FIG. 48 is a STEM-EDS micrographs of dextran-coated iron oxide nanoparticle cluster shells on gold nanocluster cores. FIG. 47 is a TEM image of 1.91:1 Fe/Au ratio and FIG. 48 is a STEM-EDS image of single nanocluster (1.91:1 Fe/Au) with iron domain in red and gold domain in green. FIGS. 49A and 49B are tables of gold nanocluster cores and various initial and final iron oxide to gold ratios. The gold was covered with lysine and citrate ligands, (zwitterionic) whereas the iron oxide particles were coated with citrate (negatively charged) ligands and dextran.

FIG. 50 is a TEM image and FIG. 51 is a STEM-EDS micrographs of citrate-coated iron oxide nanoparticle cluster shells on gold nanocluster cores. FIG. 50 is a TEM image of 0.232:1 Fe/Au ratio and FIG. 51 is a STEM-EDS image of single nanocluster (0.232:1 Fe/Au) with iron domain in green and gold domain in red.

Results for dextran-coated iron oxide are shown in FIG. 47-48 and in the Table of FIG. 49A, while results for citrate-coated iron oxide primary particles are shown in FIGS. 50 and 51 and in the Table of FIG. 49B. The extinction coefficients were large at 750 nm, indicating a large effect of the close gold spacing in the cores. TEM micrographs of the resulting multicomponent nanoclusters are also shown in FIG. 47 and FIG. 50, with the dark areas corresponding to gold cores and the lighter particles corresponding to iron oxide particles. The presence of gold and iron on the visualized particles is confirmed with STEM-EDS analysis, as shown in FIG. 48 and FIG. 51.

Rabbits were first euthanized with phenobarbital by intraperitoneal anesthesia. Thoracic aortas were then harvested under sterile conditions and washed twice with sterile PBS. Adventitia was mechanically removed and aorta was longitudinally opened. To isolate endothelial cells, the aorta was immersed in 0.2% collagenase solution for 10 minutes and the intima was gently scraped with a scalpel blade (Note: the remaining arterial wall tissue will be used for smooth muscle cell culture, see below). Digestion was terminated with endothelial cell growth medium containing 10% FBS and cells were centrifuged at 1200 rpm for 10 minutes (cells were then resuspended in sterile PBS and centrifuged under the same conditions again). The supernatant was discarded and endothelial cells (EC) were resuspended in EC growth medium containing 10% FBS. Cells were then seeded onto 6-well plates (collagen coated) and placed in 5% CO₂, 37° C. incubator. Media was changed regularly every 3 days.

Rabbit Smooth Muscle Cell Preparation. The arterial wall tissue obtained above (see EC cell preparation) was cut into 1 mm×1 mm pieces and placed in DMEM containing 10% FBS. Explants were then seeded onto 6-well plates (collagen coated) and placed in a 5% CO2, 37° C. incubator for 2 hours (No medium). Fresh DMEM containing 10% FBS was then added and media was regularly changed every 3 days.

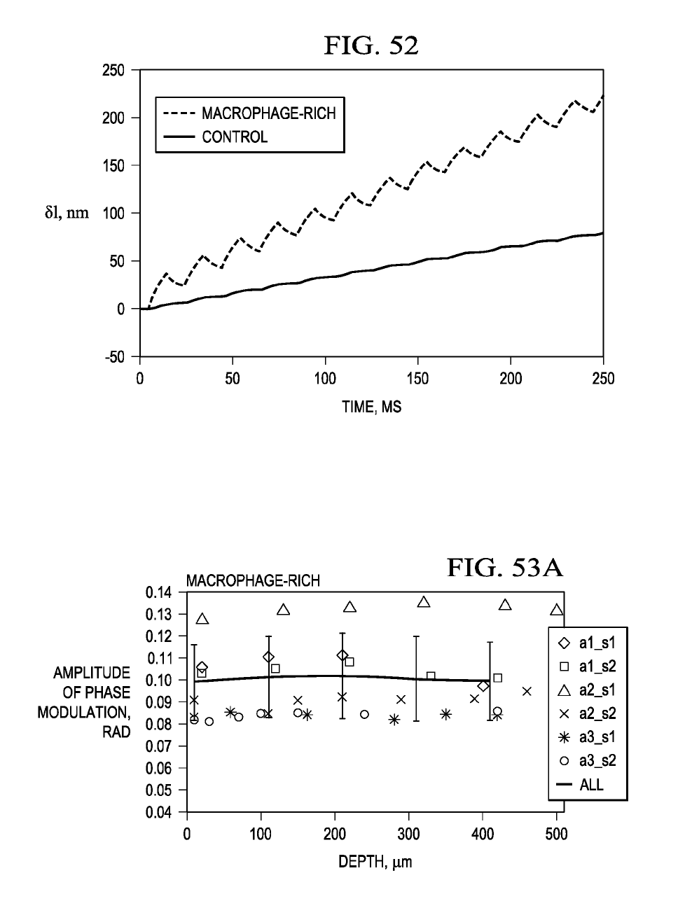

The PS-OCT system can detect nanoroses in response to laser excitation in macrophage-rich and control tissue specimens. Macrophage-rich abdominal and control thoracic aorta specimens were prepared as described above. To analyze the depth variation of optical path length modulation in a tissue specimen (macrophage-rich or control), a fast Fourier transformation (FFT) was applied to recorded δl(t) data, where t is time and peak amplitude in the frequency domain was obtained. Peak amplitude is the average modulation at the laser excitation frequency (50 Hz) and is referenced as modulation amplitude (δl(z)) at depth z in the following discussion. Modulation amplitude (δl(z)) in the macrophage-rich specimen containing nanoroses is approximately 5-fold larger than in the control specimen. FIG. 52 is an image of the time variation of thermoleastic displacement of macrophage-rich and control rabbit aortas in response to laser irradiation. A distinct modulation in thermoelastic displacement is observed in the macrophage rich abdominal aortic tissue samples. However, a similar distinct thermoelastic displacement modulation was not observed in the control thoracic aorta samples. After laser irradiation and phase-sensitive OCT measurements, histological studies were performed.

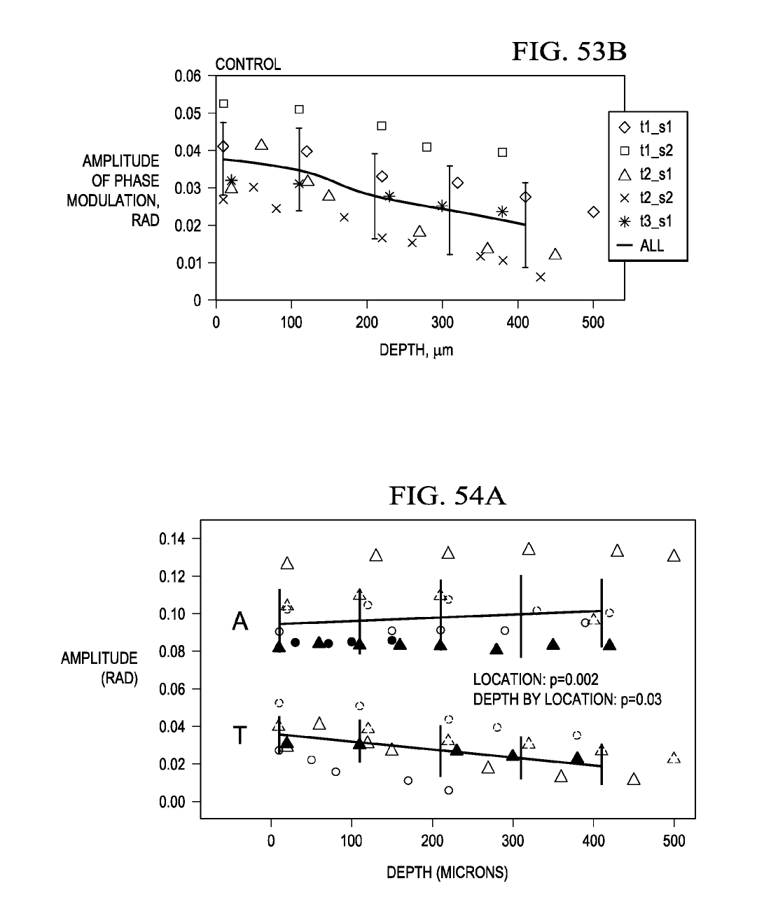

FIG. 53A is an image of the amplitude of phase modulation vs depth for control tissue specimens. FIG. 53B is an image of the amplitude of phase modulation vs depth for macrophage-rich tissue specimens. FIGS. 53A and 53B demonstrate the amplitude of the phase modulation (δl(z)) vs. depth for macrophage-rich and control tissue specimens, respectively. The depth (z) variation of the modulation amplitude (δl(z)) is distinctively different for macrophage-rich and control tissue specimens. In case of macrophage-rich tissue specimens, modulation amplitude (δl(z)) does not change significantly (<15%) with increasing tissue depth (FIG. 53A). In comparison, modulation amplitude for control tissue specimens shows a rapid decrease (more than 70%) with increasing tissue depth (FIG. 53B). Observed differences in depth-variation of the normalized modulation amplitude (δl(z)) suggests that recorded PS-OCT M-mode data is distinctly different between macrophage-rich and control tissue specimens.

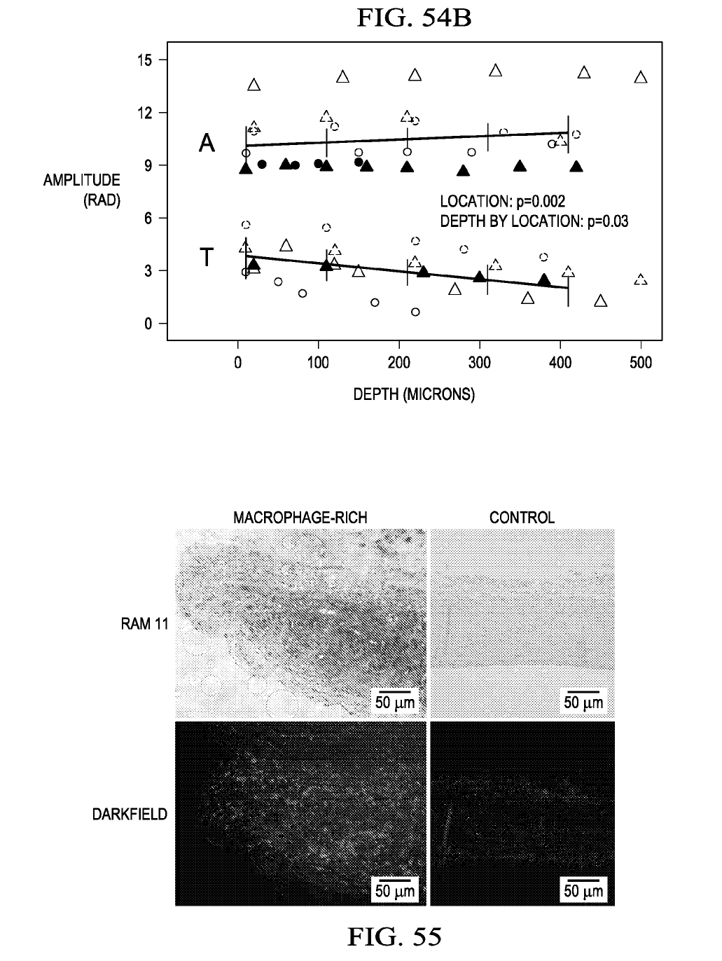

Statistical tests were additionally performed for analyzing the results of the thoracic and abdominal sections of the rabbit aorta. FIG. 54A shows the results for statistical test performed on the amplitude (in radians versus the depth in microns). Statistical testing was two-sided with a significance level of 5% and predicted values were estimated based on a repeated measures linear model in terms of location, depth, and the depth by location interaction with an autoregressive order 1 correlation assumption [SAS Version 9.1 for Windows, SAS Institute, Cary, N.C.]. For each anatomical location, whiskers extend to the predicted value plus or minus one standard error [Abdomen: Black, Thorax: Red]. The location effect (p=0.002) and the depth by location interaction (p=0.03) were significant indicating significant variation with location in both the mean amplitude and the slope relating amplitude and depth. FIG. 54 B shows the results of statistical testing performed on amplitude (in nm) versus the depth (in mm). A two-sided test with a significance level of 5% and predicted values were estimated based on a repeated measures linear model in terms of location, depth, and the depth by location interaction with an autoregressive order 1 correlation assumption [SAS Version 9.1 for Windows, SAS Institute, Cary, N.C.]. For each anatomical location, whiskers extend to the predicted value plus or minus one standard error [Abdomen: Black, Thorax: Red]. The location effect (p=0.002) and the depth by location interaction (p=0.03) were significant indicating significant variation with location in both the mean amplitude and the slope relating amplitude and depth.

FIG. 54A is an image of the replicate amplitude (rad) and depth (microns) measurements in three rabbits measured in each of two anatomical locations [A: Abdomen (injured), T: Thorax (control)] at up to 6 different depths. Animals are identified by color (blue, green, orange) and replicates by symbol (triangle, dot). FIG. 54B is an image of the replicate amplitude (nm) and depth (microns) measurements in three rabbits measured in each of two anatomical locations [A: Abdomen (injured), T: Thorax (control)] at up to 6 different depths. Animals are identified by color (blue, green, orange) and replicates by symbol (triangle, dot). Histological images of macrophage-rich tissue sections from a double balloon-injured, fat fed New Zealand white rabbit injected with nanoroses at a dose of mg Au/kg body weight.

FIG. 55 are microscopy images of macrophage-rich and control tissue sections. Macrophage-rich (left column) and control tissue (right column) sections; Brightfield RAM-11 stained (top Row) and darkfield (bottom row) unstained microscopy images. Scale bar=50 microns. The positive RAM 11 (brown color) stain confirms an area rich in macrophages in the intimal hyperplasia of the injured abdominal aorta sections. A lack of RAM 11 staining, and thus macrophages, is observed in the control specimen. Nanoroses are identified in 610 nm longpass-filtered darkfield microscopy images as bright red reflections on a dark background. The relative lack of highly-reflective constituents native to control and macrophage-rich tissue sections coupled with enhanced reflectivity of nanoroses at wavelengths longer than 610 nm is responsible for bright areas observed in darkfield microscopy images (REF). In FIG. 55, images of macrophage-rich tissue sections are positive for nanoroses while the control tissue is negative. Taken together with the RAM 11 results the microscopy images indicate that macrophage-rich tissues contain nanoroses, while the control thoracic aorta specimens contain neither macrophages nor nanoroses and are consistent with PS-OCT M-mode phase data.

The terms “therapeutic compound,” “drug”, “active agent” and “active pharmaceutical ingredient” are used interchangeably to refer to chemical entities that display certain pharmacological effects in a body and are administered for such purpose. Non-limiting examples of therapeutic compounds include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic compounds are water-soluble, poorly water-soluble drug or a drug with a low, medium or high melting point. The therapeutic compounds may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmaceuticals, nutraceuticals, diagnostic agents, nutritional agents, and the like. The active agents listed below may be found in their native state, however, they will generally be provided in the form of a salt. The active agents listed below include their isomers, analogs and derivatives.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A nanocluster composition comprising:
two or more closely spaced nanoparticles each comprising one or more metals, metal oxides, inorganic substances, or a combination thereof; and
one or more stabilizers in contact with the two or more closely spaced nanoparticles to form a nanocluster composition in which an inorganic weight percentage is greater than 50% and the average size is below 300 nm, wherein the nanocluster composition has magnetic properties, optical properties or a combination of both and the two or more closely spaced nanoparticles are distributed throughout the cross section of the nanocluster composition and not just near the surface.

2.-3. (canceled)

4. The composition of claim 1, wherein the nanocluster composition comprises 10, 20, 25, 50, 100, 1,000, 10,000, 100,000 or up to 1,000,000 nanoparticles and an average size of about 5, 10, 15, 20, 25, 30, 35, 40, 45, 50, 60, 70, 80, 90, 100, 125, 150, 175, 200, 250 and 300 nm.

5. (canceled)

6. The composition of claim 1, wherein the two or more closely spaced nanoparticles comprise Au, Ag, Cu, Al, Pt, Fe, Ni, Co, Mn, Zn, Si, Al, FePt, MnFe2O4, Fe3O4, CoFe2O4, NiFe2O4, oxides or alloys thereof.

7.-8. (canceled)

9. The composition of claim 1, wherein the nanocluster composition further comprise one or more active pharmaceuticals selected from one or more of drugs, proteins, amino acids, peptides, antibodies, medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or combinations thereof.

10.-11. (canceled)

12. The composition of claim 1, wherein the nanocluster composition deaggregates into one or more nanoparticles with an average size of less than 15 nm, 10 nm, 7 nm, 5 nm, 3 nm, 2 nm, or 1 nm over a period of 0.2-6 hours, 6-12 hours, 12-24 hours, 1 day, 2 days, 3 days, 4 days, 5 days, 6 days, 1 week, 2 weeks, 5 weeks and 10 weeks.

13. (canceled)

14. The composition of claim 1, wherein the nanocluster composition undergoes a biodegradation triggered by a change in a pH, exposure to a media, cellular uptake, a NIR light, a visible light, an electrodynamic field, a magnetic field, a radiofrequency (RF) field, an enzyme, a chemical or combinations thereof.

15. (canceled)

16. The composition of claim 1, wherein the two or more closely spaced nanoparticles are magnetic and comprise a spin-spin relaxivity sufficiently large to provide enhanced contrast in a MRI image wherein the spin-spin relaxivity of the nanocluster composition is increased by raising a volume fraction of a magnetic material within the cluster; and a volume fraction of magnetic material is greater than 0.1, 0.2, 0.3, 0.4, 0.5 or 0.6.

17.-18. (canceled)

19. The composition of claim 1, wherein the magnetic properties, optical properties or a combination of both are selected from radio-frequency, optical, microwave, MIR and NIR irradiation.

20. (canceled)

21. The composition of claim 1, wherein the one or more stabilizers are selected from a biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

22. The composition of claim 1, wherein the nanocluster composition further comprises one or more therapeutic moieties, one or more non-therapeutic moieties or a combination that target cancer or atherosclerosis, selected from folic acid, peptides, proteins, aptamers, antibodies, small RNA molecules such as but not limited to miRNA, shRNA and siRNA, poorly water soluble drugs, anti cancer drugs or combinations thereof.

23. (canceled)

24. The composition of claim 1, wherein greater than 50% of the one or more stabilizers are in the outer 25% of the volume of the nanocluster composition and 10%, 20%, 30%, 40%, 50%, 60%, 70%, 80% or 90% of the two or more closely spaced nanoparticles are not in the outer 25% of the radius of the nanocluster composition.

25. (canceled)

26. The composition of claim 1, wherein the nanocluster composition has an absorbance in the near infrared range between 700 and 1200 nm or 700 and 850 nm with a cross section of at least 10-3, preferably 0.02 cm2/microgram of metal at a wavelength in the range of 700 to 850 nm for a metal concentration in the dispersion in the range of 0.5 to 3.0 mg/mL or an absorbance in a visible region with a cross section of at least 10-3 or 0.02 cm2/microgram of metal at a wavelength in the range of 700 to 850 nm for a metal concentration in the dispersion in the range of 0.5 to 3.0 mg/mL.

27. (canceled)

28. The composition of claim 1, further comprising a coating on at least a portion of the biodegradable nanoclusters, wherein the coating comprises nanoparticles, metals, polymers, biodegradable substances, time release coatings, or a combination thereof.

29.-38. (canceled)

39. A method forming an optionally biodegradable nanocluster composition comprising the steps of:
forming an aqueous dispersion comprising two or more nanoparticles and one or more stabilizers in a solvent; and
aggregating the two or more nanoparticles and the one or more stabilizers to form a biodegradable nanocluster composition, in which an inorganic weight percentage is greater than 50% and the average size is below 300 nm, wherein the nanocluster composition has magnetic properties, optical properties or a combination of both.

40. The method of claim 39, wherein the biodegradable nanocluster composition comprises 10, 20, 25, 50, 100, 1,000, 10,000, 100,000 or up to 1,000,000 nanoparticles with an average size of about 5, 10, 15, 20, 25, 30, 35, 40, 45, 50, 60, 70, 80, 90, 100, 125, 150, 175, 200, 250 and 300 nm.

41. (canceled)

42. The method of claim 39, wherein the two or more nanoparticles comprise Au, Ag, Cu, Al, Pt, Fe, Ni, Co, Mn, Zn, Si, Al, FePt, MnFe₂O₄, Fe₃O₄, CoFe₂O₄ NiFe₂O₄, oxides or alloys thereof.

43.-44. (canceled)

45. The method of claim 39, wherein the nanocluster composition comprises one or more active agents selected from one or more therapeutic agents, one or more non-therapeutic agents or a combination that target cancer or atherosclerosis, selected from folic acid, peptides, proteins, aptamers, antibodies, small RNA molecules such as but not limited to miRNA, shRNA and siRNA, poorly water soluble drugs, anti cancer drugs or combinations thereof.

46. The method of claim 39, wherein greater than 50% of the one or more stabilizers are in the outer 25% of the volume of the nanocluster composition, wherein the one or more stabilizers are selected from starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

47.-48. (canceled)

49. A method for imaging comprising the steps of:
providing a sample;
administering one or more biodegradable nanocluster compositions to the sample, wherein the biodegradable nanocluster composition comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size is about 5, 10, 15, 20, 25, 30, 35, 40, 45, 50, 60, 70, 80, 90, 100, 125, 150, 175, 200, 250 or 300 nm, wherein the biodegradable nanocluster composition has an absorbance in the visible region, or an absorbance in the near infrared (NIR) range between 700 and 1200 nm, or are superparamagnetic, or have a strong magnetic relaxivity, magnetization or a combination thereof; and
imaging the one or more biodegradable nanocluster compositions in the sample, wherein the biodegradable nanocluster compositions are degraded by the sample after imaging.

50. The method of claim 49, further comprising one or more active agents for treatment of the sample, wherein the effect of the treatment can be monitored, the one or more active agents can be directed to a specific site, or selectively targeted to the imaged sample.

51. (canceled)

52. The method of claim 49, wherein the imaging is a magnetic resonance imaging, an optical imaging, both magnetic and optical imaging, an optical coherence tomography, a photoacoustic tomography, ultrasound imaging, a magnetomotive ultrasound imaging and a hyperspectral microscopy.

53. (canceled)

54. The method of claim 49, wherein one or more external agents are added for the degradation of the biodegradable nanocluster and release of the imaging agent, wherein the one or more external agents are selected from magnetic fields, ultrasound techniques, RF radiation, laser heating, magnetic, optical disruption or combinations thereof.

55. (canceled)

56. The method of claim 49, wherein 20%, 30%, 40%, 50%, 60%, 70%, 80%, 90%, 99% of the one or more metals, metal oxides, inorganic substances, or a combination thereof, from the biodegradable nanoclusters clear from the body within 1 day, 1 week, 1 month and 2 months, 3 months, 4 months, 5 months, or 6 months.

57.-79. (canceled)

80. A method for treating cancer or artherosclerosis comprising the steps of:
identifying a patient in need for treatment;
administering one or more biodegradable nanocluster compositions to the sample, wherein the biodegradable nanocluster composition comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size of about 5, 10, 15, 20, 25, 30, 35, 40, 45, 50, 60, 70, 80, 90, 100, 125, 150, 175, 200, 250 and 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof, wherein greater than 50% of the one or more stabilizers are in the outer 25% of the volume of the nanocluster composition;
monitoring the uptake of the biodegradable nanocluster composition; and facilitating induced cell death by an exposure to laser, high-intensity non-coherent electromagnetic irradiation, RF irradiation, or magnetic field.

81.-83. (canceled)

84. The method of claim 80, wherein one or more external agents are added for the degradation of the biodegradable nanocluster and release of the imaging agent, wherein the one or more external agents are selected from magnetic fields, RF radiation, ultrasound techniques, laser heating, magnetic, optical disruption or combinations thereof.

85. (canceled)

86. The method of claim 80, wherein 20%, 30%, 40%, 50%, 60%, 70%, 80%, 90% or 99% of the one or more metals, metal oxides, inorganic substances, or a combination thereof, from the biodegradable nanoclusters clear from the body within 1 day, 1 week, 1 month and 2 months, 3 months, 4 months, 5 months, or 6 months.

87.-90. (canceled)

91. A method for delivering an active agent comprising the steps of:
identifying a patient in need of the active agent;
administering one or more biodegradable nanocluster compositions to the sample, wherein the biodegradable nanocluster composition comprises two or more nanoparticles each comprising one or more metals, metal oxides, inorganic substances, or a combination thereof, and one or more stabilizers in contact with the two or more nanoparticles to form a biodegradable nanocluster composition in which an inorganic weight percentage is greater than 50% and the average size of about 5, 10, 15, 20, 25, 30, 35, 40, 45, 50, 60, 70, 80, 90, 100, 125, 150, 175, 200, 250 and 300 nm, wherein the biodegradable nanocluster composition has an absorbance in the visible region, an absorbance in the near infrared (NIR) range between 700 and 1200 nm, are superparamagnetic, have a strong magnetic relaxivity, magnetization or a combination thereof; and
releasing the active agent upon biodegradation of the clusters or by heating the particles with a laser in a NIR region.

92.-95. (canceled)

96. The method of claim 91, wherein the imaging is a magnetic resonance imaging, an optical imaging, both magnetic and optical imaging, an optical coherence tomography, a photoacoustic tomography, ultrasound imaging, a magnetomotive ultrasound imaging and a hyperspectral microscopy.

97. (canceled)

98. The method of claim 91, wherein one or more external agents are added for the degradation of the biodegradable nanocluster and release of the imaging agent, wherein the one or more external agents are selected from magnetic fields, RF radiation, ultrasound techniques, laser heating, magnetic, optical disruption or combinations thereof.

99. (canceled)

100. The method of claim 91, wherein 20%, 30%, 40%, 50%, 60%, 70%, 80%, 90% or 99% of the one or more metals, metal oxides, inorganic substances, or a combination thereof, from the biodegradable nanoclusters clear from the body within 1 day, 1 week, 1 month and 2 months, 3 months, 4 months, 5 months, or 6 months.

101. (canceled)

102. The method of claim 91, wherein the nanocluster composition comprises one or more active agents selected from one or more therapeutic agents, one or more non-therapeutic agents or a combination that target cancer or atherosclerosis, selected from folic acid, peptides, proteins, aptamers, antibodies, small RNA molecules such as but not limited to miRNA, shRNA and siRNA, poorly water soluble drugs, anti cancer drugs or combinations thereof and the one or more stabilizers are selected from starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

103.-179. (canceled)
